(19) United States
(12) Patent Application Publication
(10) Pub. No.: US 2022/0386606 A1
(43) Pub. Date: Dec. 8, 2022

(54) SPECIFICALLY SUBSTITUTED 3-PHENYL-5-SPIROCYCLOPENTYL-3-PYRROLIN-2-ONES AND THEIR USE AS HERBICIDES

(71) Applicant: Bayer Aktiengesellschaft, Leverkusen (DE)

(72) Inventors: Estella BUSCATO ARSEQUELL, Frankfurt am Main (DE); Alfred ANGERMANN, Kriftel (DE); Guido BOJACK, Wiesbaden-Naurod (DE); Stefan LEHR, Liederbach (DE); Elmar GATZWEILER, Bad Nauheim (DE); Elisabeth ASMUS, Hoesbach (DE)

(21) Appl. No.: 17/438,827

(22) PCT Filed: Mar. 9, 2020
(86) PCT No.: PCT/EP2020/056204

§ 371 Date: Sep. 13, 2021

(30) Foreign Application Priority Data
Mar. 15, 2019 (EP) 19163148.0

(51) Int. Cl. A01N 43/38; C07C 57/30

(52) U.S. Cl. CPC A01N 43/38; C07C 57/30

(57) ABSTRACT

The present invention relates to novel herbicidally active pyrrolin-2-ones of the general formula (I) or agrochemically acceptable salts thereof and to their use for controlling broad-leaved weeds and weed grasses in crops of useful plants.

The present invention relates to novel herbicidally active pyrrolin-2-ones of the general formula (I) or agrochemically acceptable salts thereof and to their use for controlling broad-leaved weeds and weed grasses in crops of useful plants.

The compound class of the 3-arylpyrrolidine-2,4-diones and their preparation and use as herbicides are well known from the prior art.

However, in addition, bicyclic 3-arylpyrrolidine-2,4-dione derivatives (EP-A-355 599, EP-A-415 211 and JP-A 12-053 670) and substituted monocyclic 3-arylpyrrolidine-2,4-dione derivatives (EP-A-377 893 and EP-A-442 077), for example, having herbicidal, insecticidal or fungicidal activity have also been described.

Alkynyl-substituted 3-phenylpyrrolidine-2,4-diones with a herbicidal effect are also known from WO 96/82395, WO 98/05638, WO 01/74770, WO 15/032702, WO 15/040114 or WO 16/207097.

The effectiveness of these herbicides against harmful plants is dependent on numerous parameters, for example on the application rate used, the preparation form (formulation), the harmful plants to be controlled in each case, the spectrum of harmful plants, the climate and soil conditions, and also the duration of action and/or the rate of degradation of the herbicide. In order to develop a sufficient herbicidal effect, numerous herbicides from the group of 3-arylpyrrolidine-2,4-diones require high application rates and/or have only a narrow weed spectrum, which makes their application economically unattractive. There is therefore the need for alternative herbicides which have improved properties and are economically attractive and simultaneously efficient.

Consequently, the object of the present invention is to provide novel compounds which do not have the stated disadvantages.

The present invention therefore relates to spirocyclopentylpyrrolin-2-ones of the general formula (I)

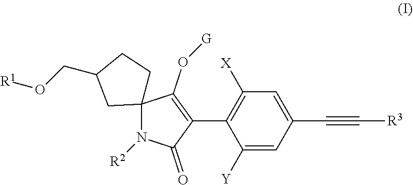

and their agrochemically acceptable salts in which
X is C₁-C₆-alkoxy, C₁-C₆-haloalkoxy or halogen,
Y is C₁-C₆-alkyl, C₁-C₆-haloalkyl, C₁-C₆-alkoxy, C₃-C₆-cycloalkyl or halogen,
R¹ is hydrogen, C₁-C₆-alkyl, C₁-C₄-alkoxy-C₂-C₄-alkyl, C₃-C₆-cycloalkyl, C₃-C₆-cycloalkyl-C₁-C₂-alkyl, C₁-C₆-haloalkyl, C₂-C₆-alkenyl, C₂-C₄-haloalkenyl, C₂-C₆-alkynyl or C₂-C₆-haloalkynyl,
R² is hydrogen, C₁-C₆-alkyl, C₁-C₄-alkoxy-C₁-C₄-alkyl, C₁-C₆-haloalkyl, C₃-C₆-cycloalkyl, C₂-C₆-alkenyl, C₂-C₆-haloalkenyl, C₂-C₆-alkynyl, C₁-C₆-alkoxy or C₁-C₆-haloalkoxy,
R³ is hydrogen, C₁-C₄-alkyl, C₁-C₄-haloalkyl, C₃-C₆-cycloalkyl or halogen,
G is hydrogen, a leaving group L or a cation E,
L is one of the radicals below

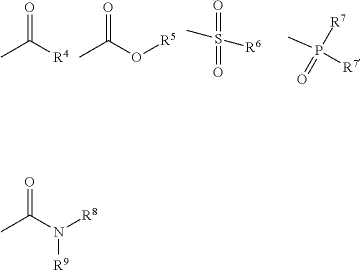

where
R⁴ represents (C₁-C₄)-alkyl or (C₁-C₃)-alkoxy-(C₂-C₄)-alkyl;
R⁵ represents (C₁-C₄)-alkyl;
R⁶ represents (C₁-C₄)-alkyl, unsubstituted phenyl or phenyl which is mono- or polysubstituted by halogen,
   (C₁-C₄)-alkyl, (C₁-C₄)-haloalkyl, (C₁-C₄)-alkoxy, (C₁-C₄)-haloalkoxy, nitro or cyano,
R⁷, R^{7′} independently of one another represent methoxy or ethoxy;
R⁸, R⁹ each independently of one another represent methyl, ethyl, phenyl or together form a saturated 5-, 6- or 7-membered ring, or together form a saturated 5-, 6- or 7-membered heterocycle having an oxygen or sulfur atom,
E represents an alkali metal ion, one ion equivalent of an alkaline earth metal, one ion equivalent of aluminum or one ion equivalent of a transition metal or a magnesium-halogen cation; represents an ammonium ion in which optionally one, two, three or all four hydrogen atoms may be replaced by identical or different radicals from the C₁-C₁₀-alkyl or C₃-C₇-cycloalkyl groups, where these independently of one another may each be mono- or polysubstituted by fluorine, chlorine, bromine, cyano, hydroxy or interrupted by one or more oxygen or sulfur atoms; represents a cyclic secondary or tertiary aliphatic or heteroaliphatic ammonium ion, for example in each case morpholinium, thiomorpholinium, piperidinium, pyrolidinium, or in each case protonated 1,4-diazabicyclo[1.1.2]octane (DABCO) or 1,5-diazabicyclo[4.3.0]undec-7-ene (DBU); represents a heteroaromatic ammonium cation, for example in each case protonated pyridine, 2-methylpyridine, 3-methylpyridine, 4-methylpyridine, 2,4-dimethylpyridine, 2,5-dimethylpyridine, 2,6-dimethylpyridine, 5-ethyl-2-methylpyridine, collidine, pyrrole, imidazole, quinoline, quinoxaline, 1,2-dimethylimidazole, 1,3-dimethylimidazolium methylsulfate; or else may further represent a trimethylsulfonium ion.

Alkyl means saturated straight-chain or branched hydrocarbyl radicals having the number of carbon atoms specified in each case, e.g. (C₁-C₆)-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Halogen-substituted alkyl means straight-chain or branched alkyl groups where some or all of the hydrogen atoms in these groups may be replaced by halogen atoms, e.g. C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl.

Alkenyl means unsaturated straight-chain or branched hydrocarbyl radicals having the number of carbon atoms stated in each case and one double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

Alkynyl means straight-chain or branched hydrocarbyl radicals having the number of carbon atoms specified in each case and one triple bond in any position, e.g. C₂-C₆-alkynyl such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

Cycloalkyl means a carbocyclic saturated ring system having preferably 3-8 ring carbon atoms, for example cyclopropyl, cyclobutyl, cyclopentyl or cyclohexyl. In the case of optionally substituted cycloalkyl, cyclic systems with substituents are included, also including substituents with a double bond on the cycloalkyl radical, for example an alkylidene group such as methylidene.

Alkoxy means saturated straight-chain or branched alkoxy radicals having the number of carbon atoms specified in each case, for example C₁-C₆-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, i-ethyl-1-methylpropoxy and 1-ethyl-2-methylpropoxy. Halogen-substituted alkoxy means straight-chain or branched alkoxy radicals having the number of carbon atoms specified in each case, where some or all of the hydrogen atoms in these groups may be replaced by halogen atoms as specified above, e.g. C₁-C₂-haloalkoxy such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-1,2-difluoroethoxy, 2,2-dichloro-2-fluoroethoxy, 2,2,2-trichloroethoxy, pentafluoroethoxy and 1,1,1-trifluoroprop-2-oxy.

The compounds of the formula (I) can, depending on the type of substituents, be present as geometric and/or optical isomers or isomer mixtures, in differing composition, also, for example, in cis or trans form, which are defined as follows:

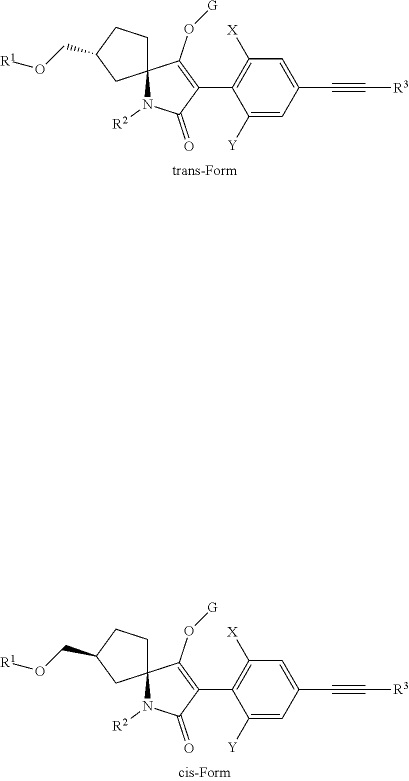

The isomer mixtures optionally obtained in the synthesis can be separated using customary techniques.

The present invention provides both the pure isomers and the tautomer and isomer mixtures, their preparation and use and compositions comprising them. However, for the sake of simplicity, the terminology used hereinbelow is always compounds of the formula (I), although both the pure compounds and also optionally mixtures with different proportions of isomeric and tautomeric compounds are intended.

The compounds of the invention are defined in general terms by the formula (I). Preferred substituents or ranges of the radicals given in the formulae mentioned above and below are illustrated hereinafter.

Preference is given to compounds of the general formula (I) in which
X is C₁-C₆-alkoxy, bromine, chlorine or fluorine,
Y is C₁-C₆-alkyl, C₁-C₆-haloalkyl, C₁-C₆-alkoxy or C₃-C₆-cycloalkyl,
R¹ is hydrogen, C₁-C₆-alkyl, C₁-C₄-alkoxy-C₂-C₄-alkyl, C₃-C₆-cycloalkyl, C₃-C₆-cycloalkyl-C₁-C₂-alkyl, C₁-C₆-haloalkyl, C₂-C₆-alkenyl, C₁-C₄-haloalkenyl, C₂-C₆-alkynyl or C₂-C₆-haloalkynyl,
R² is hydrogen, C₁-C₆-alkyl, C₁-C₄-alkoxy-C₂-C₄-alkyl, C₁-C₆-haloalkyl, C₃-C₆-cycloalkyl, C₂-C₆-alkenyl, C₂-C₆-haloalkenyl, C₂-C₆-alkynyl, C₁-C₆-alkoxy or C₁-C₆-haloalkoxy,
R³ is hydrogen, C₁-C₆-alkyl, C₁-C₄-haloalkyl, C₃-C₆-cycloalkyl or halogen,
G is hydrogen, a leaving group L or a cation E, where
L one of the radicals below

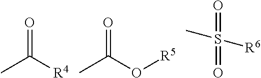

in which
R⁴ is C₁-C₄-alkyl or C₁-C₃-alkoxy-C₁-C₄-alkyl,
R⁵ is C₁-C₄-alkyl,
R⁶ is C₁-C₄-alkyl, an unsubstituted phenyl or a phenyl which is mono- or polysubstituted by halogen, C₁-C₄-alkyl, C₁-C₄-haloalkyl or cyano;
E is an alkali metal ion, one ion equivalent of an alkaline earth metal, one ion equivalent of aluminum, one ion equivalent of a transition metal, a magnesium halogen cation or an ammonium ion, in which optionally one, two, three or all four hydrogen atoms are replaced by identical or different radicals from the C₁-C₁₀-alkyl or C₃-C₇-cycloalkyl groups, each of which are independently mono- or polysubstituted by fluorine, chlorine, bromine, cyano, hydroxy.

Particular preference is given to compounds of the general formula (I) in which
X is C₁-C₆-alkoxy, bromine, chlorine or fluorine,
Y is C₁-C₆-alkyl, C₁-C₆-haloalkyl, C₁-C₆-alkoxy or C₃-C₆-cycloalkyl,
R¹ is hydrogen, C₁-C₆-alkyl, C₁-C₄-alkoxy-C₂-C₄-alkyl, C₃-C₆-cycloalkyl, C₃-C₆-cycloalkyl-C₁-C₂-alkyl, C₁-C₆-haloalkyl, C₂-C₆-alkenyl, C₁-C₄-haloalkenyl, C₂-C₆-alkynyl or C₂-C₆-haloalkynyl,
R² is hydrogen, C₁-C₆-alkyl, C₁-C₄-alkoxy-C₂-C₄-alkyl, C₁-C₆-haloalkyl, C₃-C₆-cycloalkyl, C₂-C₆-alkenyl, C₂-C₆-haloalkenyl, C₂-C₆-alkynyl, C₁-C₆-alkoxy or C₁-C₆-haloalkoxy,
R³ is hydrogen, C₁-C₄-alkyl, C₁-C₄-haloalkyl, C₃-C₆-cycloalkyl or halogen,
G is hydrogen, a leaving group L or a cation E, where
L is one of the radicals below

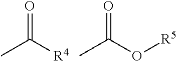

in which
R⁴ is C₁-C₄-alkyl or C₁-C₃-alkoxy-C₁-C₄-alkyl,
R⁵ is C₁-C₄-alkyl,
E is an alkali metal ion, an ion equivalent of an alkaline earth metal, an ion equivalent of aluminum, an ion equivalent of a transition metal, a magnesium halogen cation or an ammonium ion, in which optionally one, two, three or all four hydrogen atoms are replaced by identical or different radicals from the groups C₁-C₁₀-alkyl or C₃-C₇-cycloalkyl substituted are.

Very particular preference is given to compounds of the general formula (I) in which
X is methoxy, ethoxy, bromine, chlorine or fluorine,
Y is methyl, ethyl, cyclopropyl, ethoxy, methoxy,
R¹ is hydrogen, ethyl, methyl, n-propyl, n-butyl, allyl, methoxymethyl or ethoxymethyl,
R² is hydrogen or methyl,
R³ is hydrogen, methyl, ethyl, bromine,
G is hydrogen, a leaving group L or a cation E, where
L is one of the radicals below

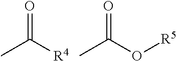

in which
R⁴ is methyl, ethyl or i-propyl,
R⁵ is methyl, ethyl, i-propyl or t-butyl,
E is a sodium ion or a potassium ion.

The preparation of the compounds of the invention of the general formula (I) is known in principle or can be effected in accordance with processes known in the literature, for example by

a) cyclizing a compound of the general formula (II)

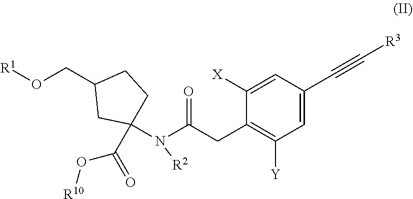

in which R¹, R², R³, X and Y have the meanings given above and R¹⁰ represents alkyl, preferably methyl or ethyl, optionally in the presence of a suitable solvent or diluent, with a suitable base, by formally eliminating the R¹⁰OH group, or
b) reacting a compound of the general formula (I)
in which R¹, R², R³, X and Y have the meanings given above, for example with a compound of the general formula (III)

Hal-L (III)

in which L has the meaning given above and Hal represents a halogen, preferably chlorine or bromine, optionally in the presence of a suitable solvent or diluent, and also a suitable base,
c) by reacting compounds of the general formula (IV)

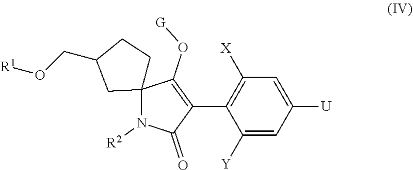

in which X, Y, R¹, R² and G have the meanings given above, and U represents a suitable leaving group, with a suitable alkynyl reagent of the general formula (V)

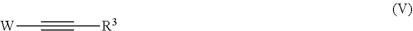

in which R³ has the meaning given above and W represents hydrogen or a suitable leaving group, optionally in the presence of suitable catalysts and a suitable base. Examples of useful leaving groups W include halogen atoms such as chlorine, bromine or iodine, alkylsulfonic ester groups, for example triflate, mesylate or nonaflate, magnesium chloride, zinc chloride, a trialkyltin radical, and boric acid radicals such as —B(OH)₂ or —B(Oalkyl)₂. Catalysts of very good suitability are especially Pd⁰ complexes, where in many cases the addition of Cu^{(I)} salts may also be very advantageous.

The described methodology is prior art and moreover also known in the literature under the heading “palladium-catalysed cross-coupling”, “Sonogashira, Negishi, Suzuki, Stille or Kumada coupling”.

Alternatively, a compound of the general formula (IV) can also be reacted with an alkynyl reagent of the general formula (VI) in an analogous application of the coupling methodology described above, then cleaved into ethynyl WO 2016/207097 PC T/EP2016/064132 compounds of the general formula (VIII) and these are finally converted with a suitable alkylating reagent to the compound (I) according to the invention, where in each case X, Y, R¹, R² and W have the meaning described and the R¹¹ group that can be cleaved off may be a trimethylsilyl group, for example.

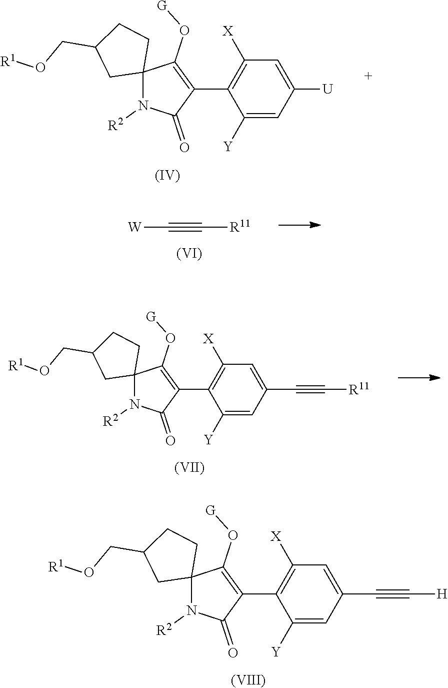

This technique, likewise known in the literature, is elucidated in detail, for example, in Beilstein Journal of Organic Chemistry 2011, 7(55), 426-431 and Catalysis Communications 2015, 60, 82-87. The precursors of the general formula (II) can be prepared analogously to known processes, for example by reacting an amino acid ester of the general formula (IX) with a phenylacetic acid of the general formula (X) where R¹, R², R³, R¹⁰, X, Y and Z have the meaning described above, optionally by adding a dehydrating agent and optionally in the presence of a suitable solvent or diluent.

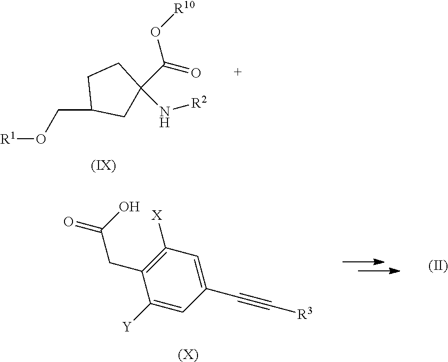

The preparation of amino acid esters of the general formula (IX) is described in principle in WO 04/024688 or WO 08/067873, and can be performed in analogy to these methods, for example by reacting a cyclopentanone of the general formula (XI) with sodium cyanide, followed by hydrolytic cleavage of the amino nitrile formed to the amino acid and esterification to give the amino ester (IX) (Strecker or Bucherer-Bergs method). The required precursors of the general formula XI can be obtained in analogy to US 20090275574 and WO 2015165279 for example.

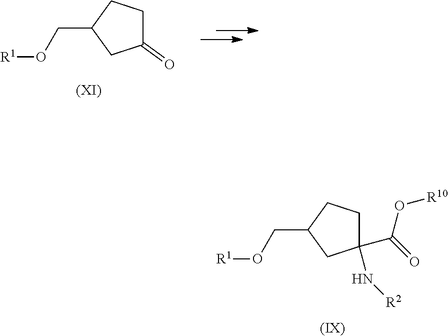

Phenylacetic acids of the general formula (X) are likewise known from WO 2015/040114 inter alia or can be prepared in analogy to literature methods, for example by reacting a compound of the general formula (XII) in which X, Y, R¹² and U have the meaning given above by the cross-coupling methodology already described with a compound of the general formula (V) in which W has the meaning given above, and cleaving the resulting carboxylic esters (XIII) by standard methods:

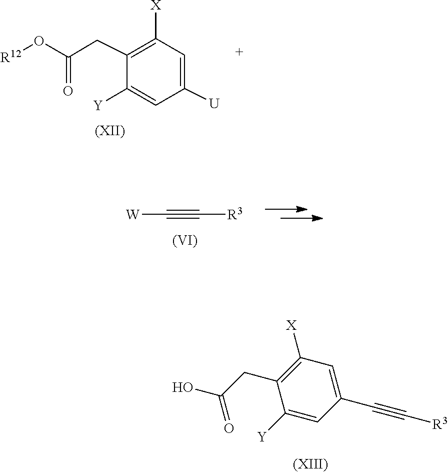

The required precursors of the general formula (XII) can be obtained, for example, by introducing an acetate unit by methods known from the literature into compounds of the general formula (XVI) in which X, Y and U have the meaning given above.

This can be accomplished, for example, analogously to the processes described in WO 05/44796 or in WO 10/115780 by Meerwein arylation of an aniline of the general formula (XIV) with vinylidene chloride followed by hydrolysis of the intermediate compound (XV) with alkoxide:

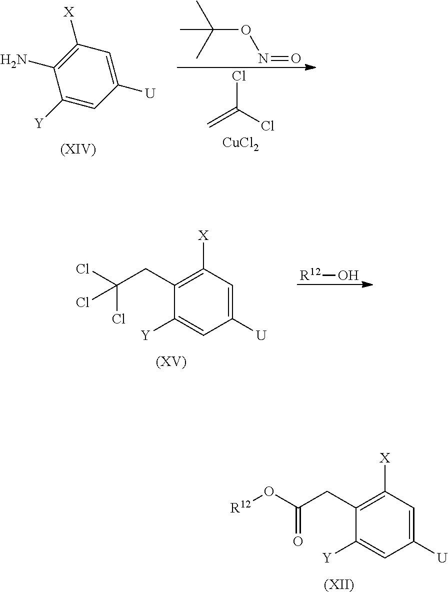

In addition, further alternative preparation processes are also known and described in WO 15/032702.

Precursors of the general formula (XIV) can be obtained in turn by customary standard methods such as bromination and/or alkylation from commercially available aminonitrophenols.

The present invention further provides compounds of the formula (X) in which the radicals have the following meanings:

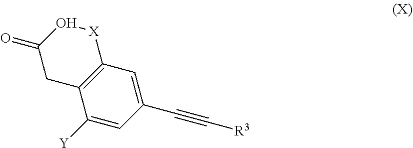

R³ is hydrogen or methyl,
X is fluorine, chlorine or bromine,
Y is methyl, ethyl, methoxy, ethoxy.

The inventive compounds of the formula (I) (and/or salts thereof), referred to collectively as “compounds of the invention” hereinafter, have excellent herbicidal efficacy against a broad spectrum of economically important monocotyledonous and dicotyledonous annual harmful plants.

The present invention therefore also provides a method for controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds of the invention are applied to the soil surface before germination, either the weed seedlings are prevented completely from emerging or the weeds grow until they have reached the cotyledon stage, but then stop growing.

If the active compounds are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

The compounds of the invention can be selective in crops of useful plants and can also be employed as non-selective herbicides.

By virtue of their herbicidal and plant growth regulatory properties, the active compounds can also be used to control harmful plants in crops of genetically modified plants which are known or are yet to be developed. In general, the transgenic plants are characterized by particular advantageous properties, for example by resistances to certain active ingredients used in the agrochemical industry, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further particular properties lie in tolerance or resistance to abiotic stress factors, for example heat, cold, drought, salinity and ultraviolet radiation.

Preference is given to using the inventive compounds of the formula (I) or salts thereof in economically important transgenic crops of useful and ornamental plants.

The compounds of the formula (I) can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods (see, for example, EP 0221044, EP 0131624). What has been described are, for example, several cases of genetic modifications of crop plants for the purpose of modifying the starch synthesized in the plants (e.g. WO 92/011376 A, WO 92/014827 A, WO 91/019806 A), transgenic crop plants which are resistant to certain herbicides of the glufosinate type (cf., for example, EP 0242236 A, EP 0242246 A) or of the glyphosate type (WO 92/000377A) or of the sulfonylurea type (EP 0257993 A, U.S. Pat. No. 5,013,659) or to combinations or mixtures of these herbicides through “gene stacking”, such as transgenic crop plants, for example corn or soya with the trade name or the designation Optimum™ GAT™ (Glyphosate ALS Tolerant),
transgenic crop plants, for example cotton, capable of producing Bacillus thuringiensis toxins (Bt toxins), which make the plants resistant to particular pests (EP 0142924 A, EP 0193259 A),
transgenic crop plants having a modified fatty acid composition (WO 91/013972 A),
genetically modified crop plants having novel constituents or secondary metabolites, for example novel phytoalexins, which cause an increase in disease resistance (EP 0309862 A, EP 0464461 A),
genetically modified plants having reduced photorespiration, which have higher yields and higher stress tolerance (EP 0305398 A),
transgenic crop plants which produce pharmaceutically or diagnostically important proteins (“molecular pharming”),
transgenic crop plants which feature higher yields or better quality,
transgenic crop plants which are distinguished by a combination, for example of the abovementioned novel properties (“gene stacking”).

Numerous molecular biology techniques which can be used to produce novel transgenic plants with modified properties are known in principle; see, for example, I. Potrykus and G. Spangenberg (eds), Gene Transfer to Plants, Springer Lab Manual (1995), Springer Verlag Berlin, Heidelberg or Christou, “Trends in Plant Science” 1 (1996) 423-431).

For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. To join the DNA fragments with one another, adapters or linkers can be placed onto the fragments, see, for example, Sambrook et al., 1989, Molecular Cloning, A Laboratory Manual, 2nd ed. Cold Spring Harbor Laboratory Press, Cold Spring Harbor, N.Y.; or Winnacker “Gene und Klone” [Genes and Clones], VCH Weinheim 2nd edition 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants. Thus, transgenic plants can be obtained whose properties are altered by overexpression, suppression or inhibition of homologous (=natural) genes or gene sequences or expression of heterologous (=foreign) genes or gene sequences.

The compounds (I) of the invention can be used with preference in transgenic crops which are resistant to growth regulators, for example 2,4-D, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active compounds, or to any desired combinations of these active compounds.

The compounds of the invention can be used with particular preference in transgenic crop plants which are resistant to a combination of glyphosates and glufosinates, glyphosates and sulfonylureas or imidazolinones. Most preferably, the compounds of the invention can be used in transgenic crop plants such as corn or soya with the trade name or the designation Optimum™ GAT™ (glyphosate ALS tolerant), for example.

When the active compounds of the invention are employed in transgenic crops, not only do the effects towards harmful plants to be observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the formula (I) as herbicides for controlling harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. These individual formulation types are known in principle and are described, for example, in: Winnacker-Küchler, “Chemische Technologie [Chemical Technology]”, Volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The formulation auxiliaries required, such as inert materials, surfactants, solvents and further additives, are likewise known and are described, for example, in: Watkins, “Handbook of Insecticide Dust Diluents and Carriers”, 2nd Ed., Darland Books, Caldwell N.J.; H. v. Olphen, “Introduction to Clay Colloid Chemistry”, 2nd ed., J. Wiley & Sons, N.Y.; C. Marsden, “Solvents Guide”, 2nd ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflachenaktive Äthylenoxid-addukte” [Interface-active Ethylene Oxide Adducts], Wiss. Verlagsgesell., Stuttgart 1976; Winnacker-Küchler, “Chemische Technologie”, volume 7, C. Hanser Verlag Munich, 4th Ed. 1986.

On the basis of these formulations, it is also possible to produce combinations with other active compounds, for example insecticides, acaricides, herbicides, fungicides, and also with safeners, fertilizers and/or growth regulators, for example in the form of a finished formulation or as a tank mix.

Active compounds which can be employed in combination with the compounds of the invention in mixed formulations or in a tank mix are, for example, known active compounds which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem H or protoporphyrinogen oxidase, as described, for example, in Weed Research 26 (1986) 441-445 or “The Pesticide Manual”, 16th edition, The British Crop Protection Council and the Royal Soc. of Chemistry, 2006 and the literature cited therein. Known herbicides or plant growth regulators which can be combined with the compounds of the invention are, for example, the following, where said active compounds are designated either with their “common name” in accordance with the International Organization for Standardization (ISO) or with the chemical name or with the code number. They always encompass all the use forms, for example acids, salts, esters and also all isomeric forms such as stereoisomers and optical isomers, even if they are not mentioned explicitly.

Examples of such herbicidal mixing partners are:

acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-5-fluoro-6-(7-fluoro-1H-indol-6-yl)pyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bixlozone, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, 1-{2-chloro-3-[(3-cyclopropyl-5-hydroxy-1-methyl-1H-pyrazol-4-yl)carbonyl]-6-(trifluoromethyl)phenyl}piperidin-2-one, 4-{2-chloro-3-[(3,5-dimethyl-1H-pyrazol-1-yl)methyl]-4-(methylsulfonyl)benzoyl}-1,3-dimethyl-1H-pyrazol-5-yl 1,3-dimethyl-1H-pyrazole-4-carboxylate, chlorfenac, chlorfenac-sodium, chlorfenprop, chlorflurenol, chlorflurenol-methyl, chloridazon, chlorimuron, chlorimuron-ethyl, 2-[2-chloro-4-(methylsulfonyl)-3-(morpholin-4-ylmethyl)benzoyl]-3-hydroxycyclohex-2-en-1-one, 4-{2-chloro-4-(methylsulfonyl)-3-[(2,2,2-trifluoroethoxy)methyl]benzoyl}-1-ethyl-1H-pyrazol-5-yl 1,3-dimethyl-1H-pyrazole-4-carboxylate, chlorophthalim, chlorotoluron, chlorthal-dimethyl, chlorsulfuron, 3-[5-chloro-4-(trifluoromethyl)pyridin-2-yl]-4-hydroxy-1-methylimidazolidin-2-one, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyranil, cyclopyrimorate, cyclosulfamuron, cycloxydim, cyhalofop, cyhalofop-butyl, cyprazine, 2,4-D, 2,4-D-butotyl, -butyl, -dimethylammonium, -diolamin, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, 3-(2,6-dimethylphenyl)-6-[(2-hydroxy-6-oxocyclohex-1-en-1-yl)carbonyl]-1-methylquinazoline-2,4(1H,3H)-dione, 1,3-dimethyl-4-[2-(methylsulfonyl)-4-(trifluoromethyl)benzoyl]-1H-pyrazol-5-yl 1,3-dimethyl-1H-pyrazole-4-carboxylate, dimetrasulfuron, dinitramine, dinoterb, diphenamid, diquat, diquat-dibromid, dithiopyr, diuron, DMPA, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, ethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, F-9960, F-5231, i.e. N-[2-chloro-4-fluoro-5-[4-(3-fluorpropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, florpyrauxifen, florpyrauxifen-benzyl, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-(2,4-dimethyl-6-nitrophenyl)-O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, 4-hydroxy-1-methoxy-5-methyl-3-[4-(trifluoromethyl)pyridin-2-yl]imidazolidin-2-one, 4-hydroxy-1-methyl-3-[4-(trifluoromethyl)pyridin-2-yl]imidazolidin-2-one, (5-hydroxy-1-methyl-1H-pyrazol-4-yl)(3,3,4-trimethyl-1,1-dioxido-2,3-dihydro-1-benzothiophen-5-yl)methanone, 6-[(2-hydroxy-6-oxocyclohex-1-en-1-yl)carbonyl]-1,5-dimethyl-3-(2-methylphenyl)quinazoline-2,4(1H,3H)-dione, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and -sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, 2-({2-[(2-methoxyethoxy)methyl]-6-(trifluoromethyl)pyridin-3-yl}carbonyl)cyclohexane-1,3-dione, methyl isothiocyanate, 1-methyl-4-[(3,3,4-trimethyl-1,1-dioxido-2,3-dihydro-1-benzothiophen-5-yl)carbonyl]-1H-pyrazol-5-yl propane-1-sulfonate, metobromuron, metolachlor, S-metolachlor, metosulam, metoxuron, metribuzin, metsulfuron, metsulfuron-methyl, molinat, monolinuron, monosulfuron, monosulfuron ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxotrione (lancotrione), oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, QYM-201, QYR-301, rimsulfuron, saflufenacil, sethoxydim, siduron, simazine, simetryn, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, tetflupyrolimet, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vemolate, ZJ-0862, i.e. 3,4-dichloro-N-{2-[(4,6-dimethoxypyrimidin-2-yl)oxy]benzyl}aniline.

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenolate mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Safeners which can be used in combination with the inventive compounds of the formula (I) and optionally in combinations with further active ingredients such as insecticides, acaricides, herbicides, fungicides as listed above are preferably selected from the group consisting of:

S1) Compounds of the formula (S1)

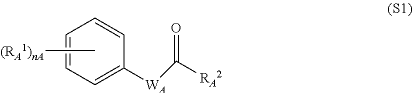

where the symbols and indices are defined as follows:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group of the partially unsaturated or aromatic five-membered heterocycles having 1 to 3 ring heteroatoms from the N and O group, where at least one nitrogen atom and at most one oxygen atom is present in the ring, preferably a radical from the group of (W_{A}¹) to (W_{A}⁴),

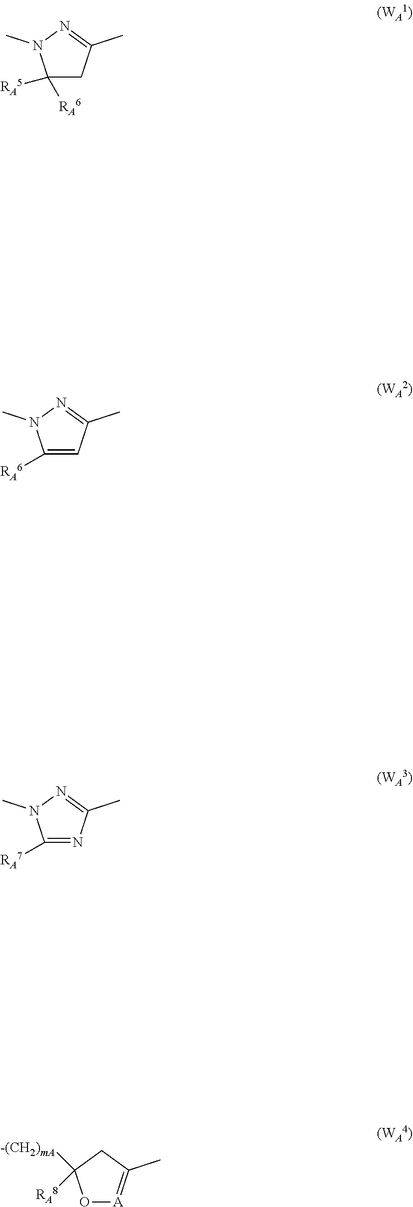

m_{A} is 0 or 1;
R_{A}² is OR_{A}³, SR_{A}³ or NR_{A}³R or a saturated or unsaturated 3- to 7-membered heterocycle having at least one nitrogen atom and up to 3 heteroatoms, preferably from the group consisting of O and S, which is joined to the carbonyl group in (S1) via the nitrogen atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;
R_{A}³ is hydrogen or an unsubstituted or substituted aliphatic hydrocarbon radical, preferably having a total of 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₈)-alkyl, (C₁-C₈)-haloalkyl, (C₁-C₄)-alkoxy-(C₁-C₈)-alkyl, cyano or COOR_{A}⁹, where
R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are identical or different and are hydrogen, (C₁-C₈)-alkyl, (C₁-C₈)-haloalkyl, (C₃-C₁₂)-cycloalkyl or substituted or unsubstituted phenyl;
preferably:
a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described in EP-A-268 554, for example;
d) compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (S1-7), and related compounds as described in EP-A-174 562 and EP-A-346 620;
e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazoline-3-carboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.

S2) Quinoline derivatives of the formula (S2)

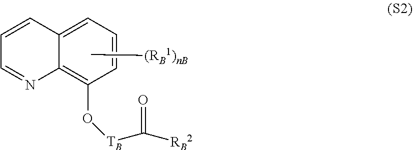

where the symbols and indices have the meanings below:
R_{B}¹ is halogen, (C₁-C₄)-alkyl, (C₁-C₄)-alkoxy, nitro or (C₁-C₄)-haloalkyl;
n_{B} is a natural number from 0 to 5, preferably from 0 to 3;
R_{B}² is OR_{B}³, SR_{B}³ or NR_{B}³R_{B}⁴ or a saturated
or unsaturated 3- to 7-membered heterocycle having at least one nitrogen atom and up to 3 heteroatoms, preferably from the group of O and S, which is joined via the nitrogen atom to the carbonyl group in (S2) and is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³;
R_{B}³ is hydrogen or an unsubstituted or substituted aliphatic hydrocarbon radical, preferably having a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁ or C₂)-alkanediyl chain which is unsubstituted or substituted by one or two (C₁-C₄)-alkyl radicals or by [(C₁-C₃)-alkoxy]carbonyl;
preferably:
a) compounds of the 8-quinolinoxyacetic acid type (S2^{a}), preferably
1-methylhexyl (5-chloro-8-quinolinoxy)acetate (“cloquintocet-mexyl”) (S2-1),
(1,3-dimethylbut-1-yl) (5-chloro-8-quinolinoxy)acetate (S2-2),
4-allyloxybutyl (5-chloro-8-quinolinoxy)acetate (S2-3),
1-allyloxyprop-2-yl (5-chloro-8-quinolinoxy)acetate (S2-4),
ethyl (5-chloro-8-quinolinoxy)acetate (S2-5),
methyl (5-chloro-8-quinolinoxy)acetate (S2-6),
allyl (5-chloro-8-quinolinoxy)acetate (S2-7),
   2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
   b) compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
   S3) Compounds of the formula (S3)

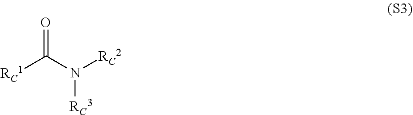

where the symbols and indices are defined as follows:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are identical or different and are hydrogen, (C₁-C₄)alkyl, (C₂-C₄)alkenyl, (C₂-C₄)alkynyl, (C₁-C₄)haloalkyl, (C₂-C₄)haloalkenyl, (C₁-C₄)alkylcarbamoyl-(C₁-C₄)alkyl, (C₂-C₄)alkenylcarbamoyl-(C₁-C₄)alkyl, (C₁-C₄)alkoxy-(C₁-C₄)alkyl, dioxolanyl-(C₁-C₄)alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;
preferably:
active compounds of the dichloroacetamide type, which are frequently used as pre-emergence safeners (soil-acting safeners), for example
   “dichlormid” (N,N-diallyl-2,2-dichloroacetamide) (S3-1),
   “R-29148” (3-dichloroacetyl-2,2,5-trimethyl-1,3-oxazolidine) from Stauffer (S3-2),
   “R-28725” (3-dichloroacetyl-2,2-dimethyl-1,3-oxazolidine) from Stauffer (S3-3),
   “benoxacor” (4-dichloroacetyl-3,4-dihydro-3-methyl-2H-1,4-benzoxazine) (S3-4),
   “PPG-1292” (N-allyl-N-[(1,3-dioxolan-2-yl)methyl]dichloroacetamide) from PPG Industries (S3-5),
   “DKA-24” (N-allyl-N-[(allylaminocarbonyl)methyl]dichloroacetamide) from Sagro-Chem (S3-6),
   “AD-67” or “MON 4660” (3-dichloroacetyl-1-oxa-3-azaspiro[4.5]decane) from Nitrokemia or Monsanto (S3-7),
   “TI-35” (1-dichloroacetylazepane) from TRI-Chemical RT (S3-8),
   “diclonon” (dicyclonon) or “BAS145138” or “LAB145138” (S3-9)
   ((RS)-1-dichloroacetyl-3,3,8a-trimethylperhydropyrrolo[1,2-a]pyrimidin-6-one) from BASF,
   “furilazole” or “MON 13900” ((RS)-3-dichloroacetyl-5-(2-furyl)-2,2-dimethyloxazolidine) (S3-10);
   and the (R) isomer thereof (S3-11).

S4) N-acylsulfonamides of the formula (S4) and salts thereof,

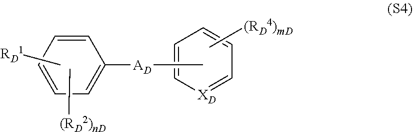

in which the symbols and indices are defined as follows:
A_{D} is SO₂—NR_{D}³-CO or CO—NR_{D}³-SO₂

### X_{D} is CH or N;

R_{D}¹ is CO—NR_{D}⁵R_{D}⁶ or NHCO—R_{D}⁷;
R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group consisting of nitrogen, oxygen and sulfur, where the seven latter radicals are substituted by v_{D} substituents from the group consisting of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three latter radicals are substituted by v_{D} radicals from the group consisting of halogen, hydroxyl, (C₁-C₄)-alkyl, (C₁-C₄)-alkoxy and (C₁-C₄)-alkylthio, or
R_{D}⁵ and R_{D}⁶ together with the nitrogen atom carrying them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
n_{D} is 0, 1 or 2;
m_{D} is 1 or 2;
v_{D} is 0, 1, 2 or 3;
among these, preference is given to compounds of the N-acylsulfonamide type, for example of the formula (S4^{a}) below, which are known, for example, from WO-A-97/45016

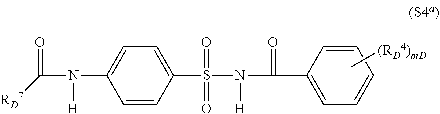

in which
R_{D}⁷ is (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
R_{D}⁴ is halogen, (C₁-C₄)-alkyl, (C₁-C₄)-alkoxy, CF₃;
m_{D} is 1 or 2;
v_{D} is 0, 1, 2 or 3;
and also
acylsulfamoylbenzamides, for example of the formula (S4^{b}) below, which are known, for example, from WO-A-99/16744,

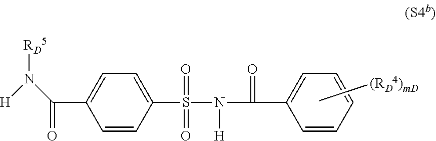

e.g. those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5)
and also
compounds of the N-acylsulfamoylphenylurea type of the formula (S4), which are known, for example, from EP-A-365484,

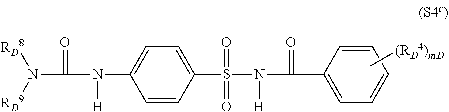

in which
R_{D}⁸ and R_{D}⁹ independently represent hydrogen, (C₁-C₅)-alkyl, (C₃-C₅)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃,
m_{D} is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea,
   and also
   N-phenylsulfonylterephthalamides of the formula (S4d), which are known, for example, from CN 101838227,

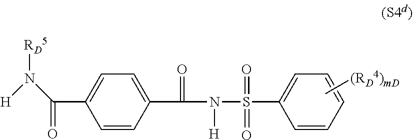

e.g. those in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.

S5) Active compounds from the class of the hydroxyaromatics and the aromatic-aliphatic carboxylic acid derivatives (S5), for example

ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.

S6) Active compounds from the class of the 1,2-dihydroquinoxalin-2-ones (S6), for example

1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.

S7) Compounds of the formula (S7), as described in WO-A-1998/38856,

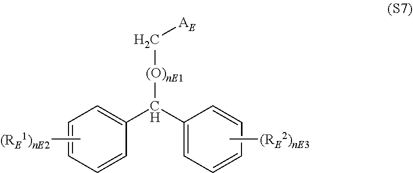

in which the symbols and indices are defined as follows:
R_{E}¹, R_{E}² are independently halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;
A_{E} is COOR_{E}³ or COSR_{E}⁴
R_{E}³, R_{E}⁴ are independently hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,
n_{E}¹ is 0 or 1
n_{E}², n_{E}³ independently of one another are 0, 1 or 2,
preferably:
diphenylmethoxyacetic acid,
ethyl diphenylmethoxyacetate,
methyl diphenylmethoxyacetate (CAS reg. no. 41858-19-9) (S7-1).

S8) Compounds of the formula (S8), as described in WO-A-98/27049,

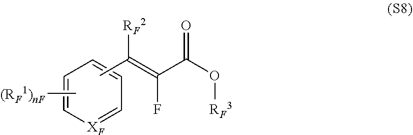

in which

### X_{F} is CH or N,

n_{F} in the case that X_{F}=N is an integer from 0 to 4 and
in the case that X_{F}=CH is an integer from 0 to 5,
   R_{F}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
   R_{F}² is hydrogen or (C₁-C₄)-alkyl,
   R_{F}³ is hydrogen, (C₁-C₅)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy; or salts thereof,
   preferably compounds in which

### X_{F} is CH,

n_{F} is an integer from 0 to 2,
R_{F}¹ is halogen, (C₁-C₄)-alkyl, (C₁-C₄)-haloalkyl, (C₁-C₄)-alkoxy, (C₁-C₄)-haloalkoxy,
R_{F}² is hydrogen or (C₁-C₄)-alkyl,
R_{F}³ is hydrogen, (C₁-C₅)-alkyl, (C₂-C₄)-alkenyl, (C₂-C₄)-alkynyl or aryl, where each of the abovementioned carbon-containing radicals is unsubstituted or substituted by one or more, preferably up to three identical or different radicals from the group consisting of halogen and alkoxy,
or salts thereof.

S9) Active compounds from the class of the 3-(5-tetrazolylcarbonyl)-2-quinolones (S9), for example

1,2-dihydro-4-hydroxy-1-ethyl-3-(5-tetrazolylcarbonyl)-2-quinolone (CAS reg. no. 219479-18-2),
1,2-dihydro-4-hydroxy-1-methyl-3-(5-tetrazolylcarbonyl)-2-quinolone (CAS Reg. No. 95855-00-8), as described in WO-A-1999/000020.

S10) Compounds of the formulae (S10^{a}) or (S10^{a})
as described in WO-A-2007/023719 and WO-A-2007/023764

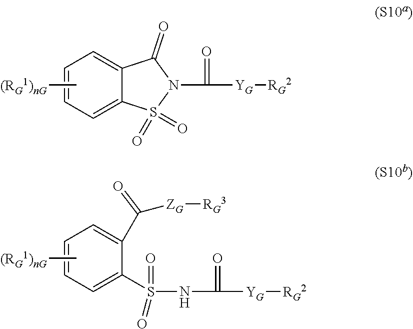

in which
R_{G}¹ is halogen, (C₁-C₄)-alkyl, methoxy, nitro, cyano, CF₃, OCF₃,
Y_{G}, Z_{G} independently of one another represent O or S,
n_{G} is an integer from 0 to 4,
R_{G}² is (C₁-C₁₆)-alkyl, (C₂-C₆)-alkenyl, (C₃-C₆)-cycloalkyl, aryl; benzyl, halobenzyl,
R_{G}³ is hydrogen or (C₁-C₆)-alkyl.

S11) Active ingredients of the oxyimino compounds type (S11), which are known as seed-dressing agents, for example

“oxabetrinil” ((Z)-1,3-dioxolan-2-ylmethoxyimino(phenyl)acetonitrile) (S11-1), which is known as a seed-dressing safener for millet/sorghum against metolachlor damage,
“fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and
“cyometrinil” or “CGA-43089” ((Z)-cyanomethoxyimino(phenyl)acetonitrile) (S11-3), which is known as a seed-dressing safener for millet/sorghum against metolachlor damage.

S12) Active compounds from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.

S13) One or more compounds from group (S13):

“naphthalic anhydride” (1,8-naphthalenedicarboxylic anhydride) (S13-1), which is known as a seed-dressing safener for corn against thiocarbamate herbicide damage,
“fenclorim” (4,6-dichloro-2-phenylpyrimidine) (S13-2), which is known as a safener for pretilachlor in sown rice,
“flurazole” (benzyl 2-chloro-4-trifluoromethyl-1,3-thiazole-5-carboxylate) (S13-3), which is known as a seed-dressing safener for millet/sorghum against alachlor and metolachlor damage,
“CL 304415” (CAS Reg. No. 31541-57-8)
(4-carboxy-3,4-dihydro-2H-1-benzopyran-4-acetic acid) (S13-4) from American Cyanamid, which is known as a safener for corn against damage by imidazolinones,
“MG 191” (CAS Reg. No. 96420-72-3) (2-dichloromethyl-2-methyl-1,3-dioxolane) (S13-5) from Nitrokemia, which is known as a safener for corn,
“MG 838” (CAS Reg. No. 133993-74-5)
(2-propenyl 1-oxa-4-azaspiro[4.5]decane-4-carbodithioate) (S13-6) from Nitrokemia,
“disulfoton” (0,0-diethyl S-2-ethylthioethyl phosphorodithioate) (S13-7),
“dietholate” (0,0-diethyl 0-phenyl phosphorothioate) (S13-8),
“mephenate” (4-chlorophenyl methylcarbamate) (S13-9).

S14) Active compounds which, in addition to herbicidal action against harmful plants, also have safener action on crop plants such as rice, for example

“dimepiperate” or “MY 93” (S-1-methyl 1-phenylethylpiperidine-1-carbothioate), which is known as a safener for rice against damage by the herbicide molinate,
“daimuron” or “SK 23” (1-(1-methyl-1-phenylethyl)-3-p-tolylurea), which is known as a safener for rice against damage by the herbicide imazosulfuron,
“cumyluron”=“JC 940” (3-(2-chlorophenylmethyl)-1-(1-methyl-1-phenylethyl)urea, see JP-A-60087254), which is known as safener for rice against damage by some herbicides,
“methoxyphenone” or “NK 049” (3,3′-dimethyl-4-methoxybenzophenone), which is known as a safener for rice against damage by some herbicides,
“CSB” (1-bromo-4-(chloromethylsulfonyl)benzene) from Kumiai, (CAS Reg. No. 54091-06-4), which is known as a safener against damage by some herbicides in rice.

S15) Compounds of the formula (S15) or tautomers thereof

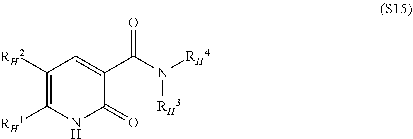

as described in WO-A-2008/131861 and WO-A-2008/131860 in which
R_{H}¹ is a (C₁-C₆)-haloalkyl radical and
R_{H}² is hydrogen or halogen and
R_{H}³, R_{H}⁴ independently of one another represent hydrogen, (C₁-C₁₆)-alkyl, (C₂-C₁₆)-alkenyl or (C₂-C₁₆)-alkynyl,
where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
where each of the 4 latter radicals is unsubstituted or substituted by one or more radicals from the group consisting of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly attached nitrogen atom represent a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.

S16) Active ingredients which are used primarily as herbicides but also have safener action on crop plants, for example
(2,4-dichlorophenoxy)acetic acid (2,4-D),
(4-chlorophenoxy)acetic acid,
(R,S)-2-(4-chloro-o-tolyloxy)propionic acid (mecoprop),
4-(2,4-dichlorophenoxy)butyric acid (2,4-DB),
(4-chloro-o-tolyloxy)acetic acid (MCPA),
4-(4-chloro-o-tolyloxy)butyric acid,
4-(4-chlorophenoxy)butyric acid,
3,6-dichloro-2-methoxybenzoic acid (dicamba),
1-(ethoxycarbonyl)ethyl 3,6-dichloro-2-methoxybenzoate (lactidichlor-ethyl).

Particularly preferred safeners are mefenpyr-diethyl, cyprosulfamide, isoxadifen-ethyl, cloquintocet-mexyl, dichlormid and metcamifen.

Wettable powders are preparations uniformly dispersible in water which, in addition to the active compound and apart from a diluent or inert substance, also comprise surfactants of ionic and/or nonionic type (wetting agent, dispersant), e.g. polyethoxylated alkylphenols, polyethoxylated fatty alcohols, polyethoxylated fatty amines, fatty alcohol polyglycolethersulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidal active compounds are finely ground, for example in customary apparatuses such as hammer mills, blower mills and air-jet mills, and simultaneously or subsequently mixed with the formulation auxiliaries.

Emulsifiable concentrates are produced by dissolving the active compound in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active compound with finely distributed solids, for example talc, natural clays, such as kaolin, bentonite and pyrophyllite, or diatomaceous earth.

Suspension concentrates may be water- or oil-based. They may be prepared, for example, by wet-grinding by means of commercial bead mills and optional addition of surfactants as have, for example, already been listed above for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be prepared either by spraying the active compound onto granular inert material capable of adsorption or by applying active compound concentrates to the surface of carrier substances, such as sand, kaolinites or granular inert material, by means of adhesives, for example polyvinyl alcohol, sodium polyacrylate or else mineral oils. Suitable active compounds can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan, fluidized-bed, extruder and spray granules, see e.g. processes in “Spray-Drying Handbook” 3rd Ed. 1979, G. Goodwin Ltd., London, J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations contain generally 0.1 to 99% by weight, especially 0.1 to 95% by weight, of compounds of the invention. In wettable powders, the active compound concentration is, for example, about 10 to 90% by weight, the remainder to 100% by weight consisting of customary formulation constituents. In emulsifiable concentrates, the active compound concentration may be about 1% to 90% and preferably 5% to 80% by weight. Formulations in the form of dusts comprise 1% to 30% by weight of active compound, preferably usually 5% to 20% by weight of active compound; sprayable solutions contain about 0.05% to 80% by weight, preferably 2% to 50% by weight of active compound. In the case of water-dispersible granules, the active compound content depends partially on whether the active compound is in liquid or solid form and on which granulation auxiliaries, fillers, etc., are used. In the water-dispersible granules, the content of active compound is, for example, between 1 and 95% by weight, preferably between 10 and 80% by weight.

In addition, the active compound formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tank mix.

For application, the formulations in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The required application rate of the compounds of the formula (I) and their salts varies according to the external conditions such as, inter alia, temperature, humidity and the type of herbicide used. It can vary within wide limits, for example between 0.001 and 10.0 kg/ha or more of active substance, but it is preferably between 0.005 and 5 kg/ha, more preferably in the range of from 0.01 to 1.5 kg/ha, particularly preferably in the range of from 0.05 to 1 kg/ha g/ha. This applies both to the pre-emergence and the post-emergence application.

A carrier is a natural or synthetic, organic or inorganic substance with which the active compounds are mixed or combined for better applicability, in particular for application to plants or plant parts or seed. The carrier, which may be solid or liquid, is generally inert and should be suitable for use in agriculture.

Useful solid or liquid carriers include: for example ammonium salts and natural rock dusts, such as kaolins, clays, talc, chalk, quartz, attapulgite, montmorillonite or diatomaceous earth, and synthetic rock dusts, such as finely divided silica, alumina and natural or synthetic silicates, resins, waxes, solid fertilizers, water, alcohols, especially butanol, organic solvents, mineral and vegetable oils, and derivatives thereof. It is likewise possible to use mixtures of such carriers. Useful solid carriers for granules include: for example crushed and fractionated natural rocks such as calcite, marble, pumice, sepiolite, dolomite, and synthetic granules of inorganic and organic meals, and also granules of organic material such as sawdust, coconut shells, corn cobs and tobacco stalks.

Suitable liquefied gaseous extenders or carriers are liquids which are gaseous at standard temperature and under atmospheric pressure, for example aerosol propellants such as halogenated hydrocarbons, or else butane, propane, nitrogen and carbon dioxide.

In the formulations, it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins, and synthetic phospholipids. Further additives may be mineral and vegetable oils.

When the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or dichloromethane, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and also water.

The compositions of the invention may additionally comprise further components, for example surfactants. Useful surfactants are emulsifiers and/or foam formers, dispersants or wetting agents having ionic or nonionic properties, or mixtures of these surfactants. Examples thereof are salts of polyacrylic acid, salts of lignosulfonic acid, salts of phenolsulfonic acid or naphthalenesulfonic acid, polycondensates of ethylene oxide with fatty alcohols or with fatty acids or with fatty amines, substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkyl sulfates, arylsulfonates, protein hydrolyzates, lignosulfite waste liquors and methylcellulose. The presence of a surfactant is necessary if one of the active compounds and/or one of the inert carriers is insoluble in water and when application is effected in water. The proportion of surfactants is between 5 and 40 percent by weight of the inventive composition. It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

If appropriate, it is also possible for other additional components to be present, for example protective colloids, binders, adhesives, thickeners, thixotropic substances, penetrants, stabilizers, sequestrants, complexing agents. In general, the active compounds can be combined with any solid or liquid additive commonly used for formulation purposes. In general, the compositions and formulations of the invention contain between 0.05 and 99% by weight, 0.01 and 98% by weight, preferably between 0.1 and 95% by weight, more preferably between 0.5 and 90% active compound, most preferably between 10 and 70 percent by weight. The active compounds or compositions of the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide coated seed, suspension concentrates, suspoemulsion concentrates, soluble concentrates, suspensions, sprayable powders, soluble powders, dusts and granules, water-soluble granules or tablets, water-soluble powders for the treatment of seed, wettable powders, natural products and synthetic substances impregnated with active compound, and also microencapsulations in polymeric substances and in coating materials for seed, and also ULV cold-fogging and warm-fogging formulations.

The formulations mentioned can be produced in a manner known per se, for example by mixing the active compounds with at least one customary extender, solvent or diluent, emulsifier, dispersant and/or binder or fixative, wetting agent, water repellent, optionally siccatives and UV stabilizers and optionally dyes and pigments, antifoams, preservatives, secondary thickeners, tackifiers, gibberellins and other processing auxiliaries.

The compositions of the invention include not only formulations which are already ready for use and can be deployed with a suitable apparatus onto the plant or the seed, but also commercial concentrates which have to be diluted with water prior to use.

The active compounds of the invention may be present as such or in their (commercial standard) formulations, or else in the use forms prepared from these formulations as a mixture with other (known) active compounds, such as insecticides, attractants, sterilants, bactericides, acaricides, nematicides, fungicides, growth regulators, herbicides, fertilizers, safeners or semiochemicals.

The inventive treatment of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for seed treatment, a water-soluble powder for slurry treatment, by incrusting, by coating with one or more coats, etc. It is furthermore possible to apply the active compounds by the ultra-low volume method or to inject the active compound preparation or the active compound itself into the soil.

As also described below, the treatment of transgenic seed with the active compounds or compositions of the invention is of particular significance. This relates to the seed of plants containing at least one heterologous gene which enables the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. This heterologous gene preferably originates from Bacillus sp., in which case the gene product is effective against the European corn borer and/or the Western corn rootworm. The heterologous gene more preferably originates from Bacillus thuringiensis.

In the context of the present invention, the inventive composition is applied to the seed alone or in a suitable formulation. Preferably, the seed is treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again.

In general, when treating the seed, it has to be ensured that the amount of the composition of the invention and/or further additives applied to the seed is chosen such that the germination of the seed is not impaired and the plant which arises therefrom is not damaged. This has to be ensured particularly in the case of active compounds which can exhibit phytotoxic effects at certain application rates.

The compositions of the invention can be applied directly, i.e. without containing any other components and without having been diluted. In general, it is preferable to apply the compositions to the seed in the form of a suitable formulation. Suitable formulations and methods for seed treatment are known to those skilled in the art and are described, for example, in the following documents: U.S. Pat. No. 4,272,417 A, 4,245,432 A, 4,808,430, 5,876,739, US 2003/0176428 A1, WO 2002/080675 A1, WO 2002/028186 A2.

The active compounds of the invention can be converted to the customary seed-dressing formulations, such as solutions, emulsions, suspensions, powders, foams, slurries or other coating compositions for seed, and also ULV formulations.

These formulations are produced in a known manner, by mixing the active compounds with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of agrochemically active compounds. Alkyl naphthalenesulfonates, such as diisopropyl or diisobutyl naphthalenesulfonates, can be used with preference.

Suitable dispersants and/or emulsifiers which may be present in the seed-dressing formulations usable in accordance with the invention are all nonionic, anionic and cationic dispersants customary for the formulation of agrochemically active compounds. Preference is given to using nonionic or anionic dispersants or mixtures of nonionic or anionic dispersants. Suitable nonionic dispersants include especially ethylene oxide/propylene oxide block polymers, alkylphenol polyglycol ethers and tristryrylphenol polyglycol ethers, and the phosphated or sulfated derivatives thereof. Suitable anionic dispersants are especially lignosulfonates, polyacrylic acid salts and arylsulfonate-formaldehyde condensates.

Antifoams which may be present in the seed-dressing formulations usable in accordance with the invention are all foam-inhibiting substances customary for the formulation of agrochemically active compounds. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances usable for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

The seed-dressing formulations usable in accordance with the invention can be used, either directly or after previously having been diluted with water, for the treatment of a wide range of different seed, including the seed of transgenic plants. In this case, additional synergistic effects may also occur in interaction with the substances formed by expression.

For the treatment of seed with the seed-dressing formulations usable in accordance with the invention or with the preparations prepared therefrom by addition of water, useful equipment is all mixing units usable customarily for seed dressing. Specifically, the seed dressing procedure is to place the seed into a mixer, to add the particular desired amount of seed-dressing formulations, either as such or after prior dilution with water, and to mix them until the formulation is distributed homogeneously on the seed. If appropriate, this is followed by a drying operation.

The active compounds of the invention, given good plant compatibility, favorable homeotherm toxicity and good environmental compatibility, are suitable for protection of plants and plant organs, for increasing harvest yields, and for improving the quality of the harvested crop. They can preferably be used as crop protection agents. They are active against normally sensitive and resistant species and also against all or specific stages of development.

Plants which can be treated in accordance with the invention include the following main crop plants: maize, soya bean, cotton, Brassica oil seeds such as Brassica napus (e.g. Canola), Brassica rapa, B. juncea (e.g. (field) mustard) and Brassica carinata, rice, wheat, sugar beet, sugar cane, oats, rye, barley, millet and sorghum, triticale, flax, grapes and various fruit and vegetables from various botanic taxa, for example Rosaceae sp. (for example pome fruits such as apples and pears, but also stone fruits such as apricots, cherries, almonds and peaches, and berry fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana trees and plantations), Rubiaceae sp. (for example coffee), Theaceae sp., Sterculiceae sp., Rutaceae sp. (for example lemons, oranges and grapefruit); Solanaceae sp. (for example tomatoes, potatoes, peppers, aubergines), Liliaceae sp., Compositae sp. (for example lettuce, artichokes and chicory—including root chicory, endive or common chicory), Umbelliferae sp. (for example carrots, parsley, celery and celeriac), Cucurbitaceae sp. (for example cucumbers—including gherkins, pumpkins, watermelons, calabashes and melons), Alliaceae sp. (for example leeks and onions), Cruciferae sp. (for example white cabbage, red cabbage, broccoli, cauliflower, Brussels sprouts, pak choi, kohlrabi, radishes, horseradish, cress and Chinese cabbage), Leguminosae sp. (for example peanuts, peas, and beans—for example runner beans and broad beans), Chenopodiaceae sp. (for example Swiss chard, fodder beet, spinach, beetroot), Malvaceae (for example okra), Asparagaceae (for example asparagus); useful plants and ornamental plants in the garden and woods; and genetically modified varieties of each of these plants.

As mentioned above, it is possible to treat all plants and their parts in accordance with the invention. In a preferred embodiment, wild plant species and plant cultivars, or those obtained by conventional biological breeding techniques, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) which have been grown by conventional breeding, by mutagenesis or by recombinant DNA techniques. They may be cultivars, varieties, biotypes and genotypes.

The treatment method of the invention can be used for the treatment of genetically modified organisms (GMOs), e.g. plants or seeds. Genetically modified plants (or transgenic plants) are plants in which a heterologous gene has been stably integrated into the genome. The term “heterologous gene” means essentially a gene which is provided or assembled outside the plant and which, upon introduction into the nuclear genome, the chloroplast genome or the mitochondrial genome, imparts to the transformed plant novel or improved agronomical or other traits because it expresses a protein or polypeptide of interest or another gene which is present in the plant, or other genes which are present in the plant are down-regulated or switched off (for example by means of antisense technology, co-suppression technology or RNAi technology [RNA interference]). A heterologous gene that is located in the genome is also called a transgene. A transgene that is defined by its specific presence in the plant genome is called a transformation or transgenic event.

Depending on the plant species or plant cultivars, their location and growth conditions (soils, climate, vegetation period, diet), the inventive treatment may also result in superadditive (“synergistic”) effects. For example, the following effects which exceed the effects actually to be expected are possible: reduced application rates and/or widened spectrum of activity and/or increased efficacy of the active compounds and compositions which can be used in accordance with the invention, better plant growth, increased tolerance to high or low temperatures, increased tolerance to drought or to water or soil salinity, increased flowering performance, easier harvesting, accelerated maturation, higher harvest yields, bigger fruits, greater plant height, greener leaf colour, earlier flowering, higher quality and/or a higher nutritional value of the harvested products, higher sugar concentration within the fruits, better storage stability and/or processability of the harvested products.

Plants and plant cultivars which are preferably treated in accordance with the invention include all plants which have genetic material which imparts particularly advantageous, useful traits to these plants (whether obtained by breeding and/or biotechnological means).

Examples of nematode-resistant plants are described, for example, in the following U.S. patent application Ser. Nos. 11/765,491, 11/765,494, 10/926,819, 10/782,020, 12/032,479, 10/783,417, 10/782,096, 11/657,964, 12/192,904, 11/396,808, 12/166,253, 12/166,239, 12/166,124, 12/166,209, 11/762,886, 12/364,335, 11/763,947, 12/252,453, 12/209,354, 12/491,396 and 12/497,221.

Plants that may be treated according to the invention are hybrid plants that already express the characteristics of heterosis, or hybrid effect, which results generally in higher yield, vigour, better health and resistance towards biotic and abiotic stress factors. Such plants are typically produced by crossing an inbred male-sterile parent line (the female crossbreeding parent) with another inbred male-fertile parent line (the male crossbreeding parent). Hybrid seed is typically harvested from the male-sterile plants and sold to growers. Male-sterile plants can sometimes (e.g. in corn) be produced by detasselling (i.e. the mechanical removal of the male reproductive organs or male flowers) but, more typically, male sterility is the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored. This can be accomplished by ensuring that the male crossbreeding parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species. However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Plants can be made tolerant to glyphosate by various methods. Thus, for example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science, 221, 370-371), the CP4 gene of the bacterium Agrobacterium sp. (Barry et al., 1992, Cuff. Topics Plant Physiol. 7, 139-145), the genes encoding a petunia EPSPS (Shah et al., 1986, Science 233, 478-481), a tomato EPSPS (Gasser et al., 1988, J. Biol. Chem. 263, 4280-4289) or an Eleusine EPSPS (WO 01/66704). It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyltransferase enzyme. Glyphosate-tolerant plants can also be obtained by selecting plants containing naturally-occurring mutations of the abovementioned genes. Plants which express EPSPS genes which impart glyphosate tolerance have been described. Plants which express other genes which impart glyphosate tolerance, for example decarboxylase genes, have been described.

Other herbicide-resistant plants are for example plants made tolerant to herbicides inhibiting the enzyme glutamine synthase, such as bialaphos, phosphinothricin or glufosinate. Such plants can be obtained by expressing an enzyme detoxifying the herbicide or a mutant of the glutamine synthase enzyme that is resistant to inhibition. One example of such an effective detoxifying enzyme is an enzyme encoding a phosphinothricin acetyltransferase (such as the bar or pat protein from Streptomyces species). Plants expressing an exogenous phosphinothricin acetyltransferase have been described.

Further herbicide-tolerant plants are also plants that have been made tolerant to the herbicides inhibiting the enzyme hydroxyphenylpyruvate dioxygenase (HPPD). Hydroxyphenylpyruvate dioxygenases are enzymes that catalyze the reaction in which para-hydroxyphenylpyruvate (HPP) is converted to homogentisate. Plants tolerant to HPPD inhibitors can be transformed with a gene encoding a naturally-occurring resistant HPPD enzyme, or a gene encoding a mutated or chimeric HPPD enzyme, as described in WO 96/38567, WO 99/24585, WO 99/24586, WO 2009/144079, WO 2002/046387 or U.S. Pat. No. 6,768,044. Tolerance to HPPD inhibitors can also be obtained by transforming plants with genes encoding certain enzymes enabling the formation of homogentisate despite inhibition of the native HPPD enzyme by the HPPD inhibitor. Such plants are described in WO 99/34008 and WO 02/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928. In addition, plants can be made more tolerant to HPPD inhibitors by inserting into the genome thereof a gene which encodes an enzyme which metabolizes or degrades HPPD inhibitors, for example CYP450 enzymes (see WO 2007/103567 and WO 2008/150473).

Other herbicide-resistant plants are plants which have been rendered tolerant to acetolactate synthase (ALS) inhibitors. Known ALS inhibitors include, for example, sulfonylurea, imidazolinone, triazolopyrimidines, pyrimidinyloxy(thio)benzoates, and/or sulfonylaminocarbonyltriazolinone herbicides. It is known that different mutations in the ALS enzyme (also known as acetohydroxy acid synthase, AHAS) confer tolerance to different herbicides and groups of herbicides, as described, for example, in Tranel and Wright (Weed Science 2002, 50, 700-712). The production of sulfonylurea-tolerant plants and imidazolinone-tolerant plants has been described. Further sulfonylurea- and imidazolinone-tolerant plants have also been described.

Further plants tolerant to imidazolinones and/or sulfonylureas can be obtained by induced mutagenesis, by selection in cell cultures in the presence of the herbicide or by mutation breeding (cf., for example, for soya beans U.S. Pat. No. 5,084,082, for rice WO 97/41218, for sugar beet U.S. Pat. No. 5,773,702 and WO 99/057965, for lettuce U.S. Pat. No. 5,198,599 or for sunflower WO 01/065922).

Plants or plant cultivars (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:

a. plants which contain a transgene capable of reducing the expression and/or the activity of the poly(ADP-ribose) polymerase (PARP) gene in the plant cells or plants;
b. plants which contain a stress tolerance-enhancing transgene capable of reducing the expression and/or the activity of the PARG-encoding genes of the plants or plant cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase.

Plants or plant cultivars (obtained by plant biotechnology methods such as genetic engineering) which may also be treated according to the invention show altered quantity, quality and/or storage stability of the harvested product and/or altered properties of specific components of the harvested product such as, for example:

1) Transgenic plants which synthesize a modified starch which, in its physicochemical characteristics, in particular the amylose content or the amylose/amylopectin ratio, the degree of branching, the average chain length, the side chain distribution, the viscosity behavior, the gelling strength, the starch granule size and/or the starch granule morphology, is changed in comparison with the synthesized starch in wild-type plant cells or plants, so that this modified starch is better suited to specific applications.
2) Transgenic plants which synthesize non-starch carbohydrate polymers or which synthesize non-starch carbohydrate polymers with altered properties in comparison to wild-type plants without genetic modification. Examples are plants which produce polyfructose, especially of the inulin and levan type, plants which produce alpha-1,4-glucans, plants which produce alpha-1,6-branched alpha-1,4-glucans, and plants producing alternan.
3) Transgenic plants which produce hyaluronan.
4) Transgenic plants or hybrid plants such as onions with particular properties, such as “high soluble solids content”, “low pungency” (LP) and/or “long storage” (LS).

Plants or plant cultivars (obtained by plant biotechnology methods such as genetic engineering) which may also be treated according to the invention are plants, such as cotton plants, with altered fiber characteristics. Such plants can be obtained by genetic transformation, or by selection of plants containing a mutation imparting such altered fiber characteristics and include:

a) plants, such as cotton plants, containing an altered form of cellulose synthase genes;
b) plants, such as cotton plants, which contain an altered form of rsw2 or rsw3 homologous nucleic acids, such as cotton plants with an increased expression of sucrose phosphate synthase;
c) plants, such as cotton plants, with increased expression of sucrose synthase;
d) plants, such as cotton plants, wherein the timing of the plasmodesmatal gating at the base of the fiber cell is altered, for example through downregulation of fiber-selective β-1,3-glucanase;
e) plants, such as cotton plants, which have fibers with altered reactivity, for example through expression of the N-acetylglucosaminetransferase gene, including nodC, and chitin synthase genes.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants containing a mutation imparting such altered oil characteristics and include:

a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Plants or plant cultivars (which can be obtained by plant biotechnology methods such as genetic engineering) which may also be treated according to the invention are plants such as potatoes which are virus-resistant, for example to the potato virus Y (SY230 and SY233 events from Tecnoplant, Argentina), or which are resistant to diseases such as potato late blight (e.g. RB gene), or which exhibit reduced cold-induced sweetness (which bear the genes Nt-Inh, II-INV) or which exhibit the dwarf phenotype (A-20 oxidase gene).

Plants or plant cultivars (obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants containing a mutation imparting such altered characteristics, and include plants such as oilseed rape with retarded or reduced seed shattering.

Particularly useful transgenic plants which can be treated according to the invention are plants with transformation events or combinations of transformation events which are the subject of granted or pending petitions for nonregulated status in the USA at the Animal and Plant Health Inspection Service (APHIS) of the United States Department of Agriculture (USDA). Information relating to this is available at any time from APHIS (4700 River Road Riverdale, Md. 20737, USA), for example via the website http://www.aphis.usda.gov/brs/not_reg.html. At the filing date of this application, the petitions with the following information were either granted or pending at APHIS:
Petition: Identification number of the petition. The technical description of the transformation event can be found in the specific petition document available from APHIS on the website via the petition number. These descriptions are hereby disclosed by reference.
Extension of a petition: Reference to an earlier petition for which an extension of scope or term is being requested.
Institution: Name of the person submitting the petition.
Regulated article: The plant species in question.
Transgenic phenotype: The trait imparted to the plant by the transformation event.
Transformation event or line: The name of the event(s) (sometimes also referred to as line(s)) for which nonregulated status is being requested.
APHIS documents: Various documents which have been published by APHIS with regard to the petition or can be obtained from APHIS on request.

Particularly useful transgenic plants which can be treated in accordance with the invention are plants which comprise one or more genes which code for one or more toxins, for example the transgenic plants which are sold under the following trade names: YIELD GARD® (for example maize, cotton, soya beans), KnockOut® (for example maize), BiteGard® (for example maize), BT-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned include maize varieties, cotton varieties and soya bean varieties which are available under the following trade names: Roundup Ready® (tolerance to glyphosates, for example corn, cotton, soya beans), Liberty Link® (tolerance to phosphinothricin, for example oilseed rape), IMI® (tolerance to imidazolinone) and SCS® (tolerance to sulfonylurea), for example corn. Herbicide-resistant plants (plants bred in a conventional manner for herbicide tolerance) which may be mentioned include the varieties sold under the name Clearfield® (for example corn).

The examples which follow illustrate the present invention.

### CHEMICAL EXAMPLES

### Example I-11 3-(2-Bromo-6-methoxy-4-prop-1-ynylphenyl)-8-(ethoxymethyl)-4-hydroxy-1-azaspiro[4.4]non-3-en-2-one

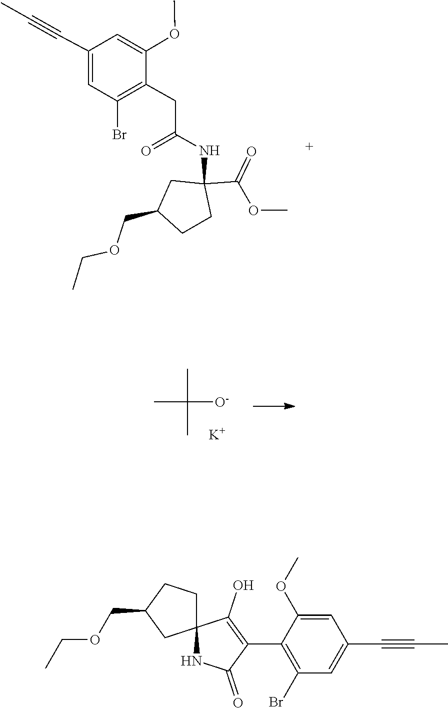

To an initial charge of 1.00 g (2.14 mmol) of methyl 1-[[2-(2-bromo-6-methoxy-4-prop-1-ynyl-phenyl)acetyl]amino]-3-(ethoxymethyl)cyclopentanecarboxylate in 3.5 ml of dimethylformamide was added 0.52 g (4.71 mmol) of potassium tert-butoxide. The mixture was stirred at room temperature for 1 h, water was added and the mixture was washed with dichloromethane and acidified with 2N aqueous hydrochloric acid. The precipitated solid was filtered off with suction.

This gave 900 mg of a yellow solid (96% yield).

### Example P-13 [3-(2-Bromo-6-methoxy-4-prop-1-ynyl-phenyl)-8-(ethoxymethyl)-2-oxo-1-azaspiro[4.4]non-3-en-4-yl] ethyl carbonate

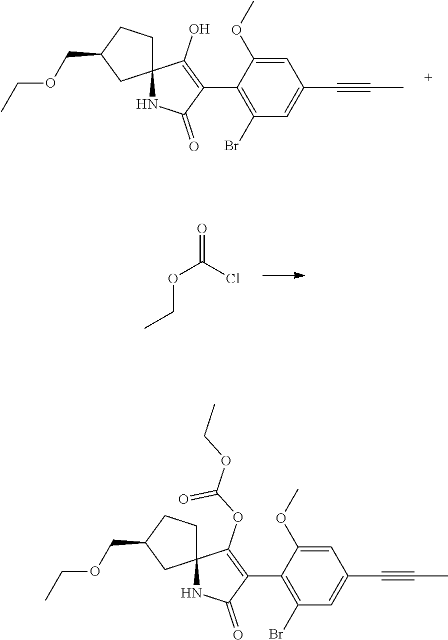

To an initial charge of 0.80 g (1.47 mmol) of 3-(2-bromo-6-methoxy-4-prop-1-ynylphenyl)-8-(ethoxymethyl)-4-hydroxy-1-azaspiro[4.4]non-3-en-2-one and 0.29 g (2.94 mmol) of triethylamine in 10 ml of methylene chloride was added 0.19 g (1.76 mmol) of ethyl chloroformate dropwise. The mixture was left to stir at room temperature for 1 h. The mixture was washed with water, dried, the solvent was distilled off, and the residue was purified by chromatography.

This gave 700 mg of a light-colored solid (93% yield).

Analogously to this example and also according to the general details relating to the preparation, the following compounds are obtained:

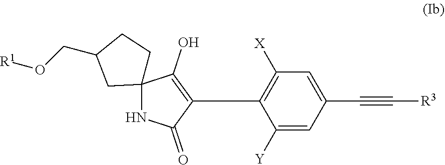

TABLE 1
Compounds of the general formula (Ib)

| Example | | | | | Stereo- | |
|---|---|---|---|---|---|---|
| No. | R¹ | R³ | X | Y | chemistry | ¹H NMR (400 MHz, δ in ppm) |
| I-01 | Me | H | OMe | Cl | cis | D₂O, NaOD δ = 1.45-1.56 (m, 2H), 1.77 (m, 1H), 2.00-2.08 (m, 2H), 2.28 (m, |
| | | | | | | 1H), 2.41-2.47 (m, 1H), 3.40 (s, 3H), 3.40 (s, 1H) 3.54 (d, 2H), 3.78 (s, 3H), 7.13 |
| | | | | | | (2, 1H), 7.31 (s, 1H) |
| I-02 | Et | Me | Me | Cl | trans | d₆-DMSO δ = 1.09-1.12 (t, 3H), 1.45-1.61 (m, 1H), 1.62-1.67 (m, 2H), 1.76-1.82 |
| | | | | | | (m 1H), 1.91-1.94 (m 1H), 2.05 (s, 3H), 2.10 (s, 3H), 3.28-3.35 (m, 2H), 3.41- |
| | | | | | | 3.45 (q, 2H), 7.22 (s, 1H), 7.27 (s, 1H), 7.93 (br, 1H), 10.98 (s, 1H) |
| I-03 | Et | Me | Et | Cl | trans | d₆-DMSO δ = 1.02 (t, 3H), 1.11 (t, 3H), 1.45-1.60 (m 1H), 1.61 (m, 2H), 1.75- |
| | | | | | | 1.81 (m, 1H), 1.91 (m, 1H), 2.05 (s, 3H), 2.07-2.13 (m 1H), 2.40-2.45 (m, 3H), |
| | | | | | | 3.33 (q, 2H), 3.43 (q, 2H), 7.21 (s, 1H), 7.28 (s, 1H), 7.92 (br, 1H), 10.96 (br, 1H) |
| I-04 | Me | Me | Et | Cl | trans | d₆-DMSO δ = 1.00-1.04 (t, 3H), 1.45-1.58 (m, 1H), 1.60-1.76 (m, 2H), 1.77-1.83 |
| | | | | | | (m, 1H), 1.91-1.99 (m, 1H), 2.05 (s, 3H), 2.10-2.15 (m, 1H), 2.43 (q, 2H), 3.25 (s, |
| | | | | | | 3H), 3.26 (m 2H), 7.21 (s, 1H), 7.29 (s, 1H), 7.93 (s, 1H), 10.96 (s, 1H) |
| I-05 | Et | Me | OMe | Br | trans | d₆-DMSO δ = 1.09-1.13 (t, 3H), 1.43-1.58 (m, 1H), 1.61-1.66 (m, 2H), 1.72-1.92 |
| | | | | | | (m, 2H), 2.06 (s, 3H), 3.29 (m, 1H), 3.39-3.43 (q, 2H), 3.69 (s, 3H), 6.98 (s, 1H), |
| | | | | | | 7.19 (s, 1H), 7.78 (s, 1H), 10.78 (s, 1H) |
| I-06 | Me | Me | Me | Cl | cis | d₆-DMSO δ = 1.45-168 (m, 3H), 1.84-1.94 (m 1H), 1.98-2.21 (m, 2H), 2.05 (s, |
| | | | | | | 3H), 2.10 (s, 3H), 2.30-2.39 (m, 1H), 3.33 (m, 2H), 7.22 (s, 1H), 7.27 (s, 1H), |
| | | | | | | 7.74 (br, 1H), 11.02 (br, 1H) |
| I-07 | Et | Me | OMe | Cl | trans | D₂O, NaOD δ δ = 1.18-1.23 (t, 3H), 1.50-1.58 (m 1H), 1.68-1.74 (m 1H), 1.80- |
| | | | | | | 1.85 (m, 2H), 2.01-2.03 (m 1H), 2.05 (s, 3H)2.09-2.16 (m 1H), 2.48-2.53 (m, |
| | | | | | | 1H), 3.49-3.54 (m 2H), 3.59-3.66 (q, 2H), 3.78 (s, 3H), 7.01 (s, 1H), 7.18 (s, 3H) |
| I-08 | Et | H | OMe | Cl | cis | D₂O, NaOD δ δ = 1.18-1.23 (t, 3H), 1.46-1.55 (m, 2H), 1.74-1.82 (m, 1H), 2.00- |
| | | | | | | 2.08 (m, 2H), 2.26-2.32 (m, 1H), 2.39-2.48 (m, 1H), 3.50-.3.57 (m, 2H), 3.60- |
| | | | | | | 3.65 (q, 2H), 3.78 (s, 3H), 7.13 (s, 1H), 7.31 (s, 1H) |
| I-09 | Et | Me | Me | Cl | cis | d₆-DMSO δ = 1.10-1.13 (t, 3H), 1.45-1.69 (m, 3H), 1.85-1.90 (m, 1H), 1.98-2.01 |
| | | | | | | (m, 1H), 2.05 (s, 3H), 2.10 (s, 3H), 2.18-2.23 (m, 1H), 2.31-2.36 (m, 1H), 3.34- |
| | | | | | | 3.37 (m, 2H), 3.38-3.46 (q, 2H), 7.22 (s, 3H), 7.27 (s, 3H), 7.72 (br, 1H) |
| I-10 | Me | Me | OMe | Br | cis | D₂O, NaOD δ δ = 1.16-1.20 (t, 3H), 1.46-1.58 (m, 2H), 1.75-1.82 (m, 1H), 2.00- |
| | | | | | | 2.04 (m, 2H), 2.05 (s, 3H), 2.25-2.40 (m, 1H), 2.42-2.48 (m, 1H), 3.39 (s, 3H), |
| | | | | | | 3.50-3.57 (m, 1H), 3.62-3.67 (q, 2H), 3.75 (s, 3H), 7.05 (s, 1H), 7.36 (s, 1H) |
| I-11 | Et | Me | OMe | Br | cis | d₆-DMSO δ = 1.09-1.13 (t, 3H), 1.44-1.52 (m, 2H), 1.54-1.62 (m 1H), 1.85-1.88 |
| | | | | | | (m, 1H), 1.96-2.02 (m, 1H), 2.06 (s, 3H), 2.18-2.34 (m, 3H), 3.33-3.46 (m, 1H), |
| | | | | | | 3.44-.3.46 (q, 2H), 3.69 (s, 3H), 6.98 (s, 1H), 7.19 (s, 1H), 7.58 (br, 1H), 10.8 (br, |
| | | | | | | 1H) |
| I-12 | Me | Me | Me | Cl | trans | d₆-DMSO δ = 1.47-1.59 (m, 1H), 1.65 (m, 2H), 1.77-1.80 (m, 1H), 1.93-1.99 (m, |
| | | | | | | 1H), 2.05 (s, 3H), 2.11 (s, 3H), 3.25 (s, 3H), 7.22 (s, 1H), 7.27 (s, 1H) |
| I-13 | Me | H | OMe | Cl | trans | D₂O, NaOD δ δ = 1.48-1.57 (m 1H), 1.69-1.85 (m, 3H), 2.00-2.14 (m, 2H), 2.46- |
| | | | | | | 2.55 (m, 1H), 3.39 (s, 3H), 3.49 (m, 2H), 3.32 (s, 3H), 7.13 (s, 1H), 7.30 (s, 1H) |
| I-14 | Et | H | OMe | Cl | trans | D₂O, NaOD δ δ = 1.20 (t, 3H), 1.53 (m, 1H), 1.69-1.92 (m, 3H), 1.99-2.16 (m, |
| | | | | | | 2H), 2.5 (m, 1H), 3.52 (m, 2H), 3.61 (q, 2H), 3.78 (s, 3H), 7.21 (s, 1H), 7.30 (s, |
| | | | | | | 1H) |
| I-15 | Et | Me | Et | Cl | cis | d₆-DMSO δ = 1.0 (t, 3H), 1.1 (t, 3H), 1.5-1.9 (m, 4H), 2.0 (s, 3H), 2.2 (m, 1H), |
| | | | | | | 2.3-2.4 (m, 2H), 3.4 (s, 3H), 3.5 (m, 4H), 7.1 (s, 1H), 7.25 (s, 1H), 7.8 (br, 1H) |
| I-16 | Me | Me | Et | Cl | cis | d₆-DMSO δ = 1.0 (t, 3H), 1.5-1.9 (m, 4H), 2.0 (s, 3H), 2.2 (m, 1H), 2.3-2.4 (m, |
| | | | | | | 2H), 3.4 (s, 3H), 3.5 (m, 4H), 7.1 (s, 1H), 7.25 (s, 1H), 7.8 (br, 1H) |
| I-17 | Et | Me | Me | Cl | cis | d₆-DMSO δ = 1.1-1.2 (t, 3H), 1.4-1.9 (m, 5H), 2.01 (s, 3H), 2.1 (s, 3H), 2.2-2.7 |
| | | | | | | (m, 2H), 3.4 (q, 2H), 3.5 (m, 2H), 7.22 (s, 1H), 7.27 (s, 1H), 7.73 (br, 1H), 7.93 |
| | | | | | | (br, 1H) |
| I-18 | Me | | Me | Br | trans | d₆-DMSO δ = 1.4 (m, 1H), 1.4 (m, 2H), 1.8-1.9 (m, 2H), 2.0 (s, 3H), 2.1 (s, 3H), |
| | | | | | | 3.2 (m, 4H), 3.3 (s, 3H), 7.25 (s, 1H), 7.44 (s, 1H), 7.92 (br, 1H), 10.9 (br, 1H) |
| I-19 | Me | Me | Me | Br | cis | d₆-DMSO δ = 1.3 (m, 2H), 1.4 (m, 1H), 1.8-1.9 (m, 1H), 2.0 (s, 3H), 2.1 (s, 3H), |
| | | | | | | 2.2 (m, 2H), 3.3 (s, 3H), 3.4 (m, 2H), 7.25 (s, 1H), 7.44 (s, 1H), 7.74 (br, 1H), |
| | | | | | | 10.9 (br, 1H) |
| I-20 | Et | Me | Me | Br | trans | d₆-DMSO δ = 1.1 (t, 3H), 1.4-1.9 (m, 5H), 2.0 (s, 3H), 2.1 (s, 3H), 2.2-2.3 (m, |
| | | | | | | 2H), 3.4 (q, 2H), 3.5 (m, 2H), 7.25 (br, 1H), 7.44 (s, 1H), 7.98 (s, 1H), 10.96 (br, |
| | | | | | | 1H) |
| I-21 | Et | Me | Me | Br | cis | d₆-DMSO δ = 1.1 (t, 3H), 1.4-1.9 (m, 4H), 2.0 (s, 3H), 2.1 (s, 3H), 2.2-2.3 (m, |
| | | | | | | 2H), 3.4 (q, 2H), 3.5 (m, 2H), 7.25 (br, 1H), 7.44 (s, 1H), 7.73 (s, 1H), 10.98 (br, |
| | | | | | | 1H) |
| I-22 | Me | Me | Et | Br | cis | d₆-DMSO δ = 1.0 (t, 3H), 1.4-1.6 (m, 3H), 1.8 (m, 1H), 2.1 (s, 3H), 2.2-2.4 (m, |
| | | | | | | 1H), 3.3 (s, 3H), 3.4 (q, 2H), 7.1 (br, 1H), 7.5 (s, 1H), 7.81 (s, 1H), 10.9 (br, 1H) |
| I-23 | Me | Me | Et | Br | trans | d₆-DMSO δ = 1.0 (t, 3H), 1.4-1.6 (m, 3H), 1.8 (m, 1H), 2.1 (s, 3H), 2.2-2.4 (m, |
| | | | | | | 1H), 3.3 (s, 3H), 3.4 (q, 2H), 7.1 (br, 1H), 7.5 (s, 1H), 7.99 (s, 1H), 10.9 (br, 1H) |
| I-24 | Et | Me | Et | Br | cis | d₆-DMSO δ = 1.1 (t, 3H), 1.2 (t, 3H), 1.4-1.7 (m, 3H), 1.8 (m, 1H), 2.1 (s, 3H), |
| | | | | | | 2.2-2.4 (m, 2H), 3.4 (q, 2H), 3.5 (q, 2H), 7.11 (br, 1H), 7.5 (s, 1H), 7.79 (s, 1H) |
| I-25 | Et | Me | Et | Br | trans | d₆-DMSO δ = 1.1 (t, 3H), 1.2 (t, 3H), 1.4-1.7 (m, 3H), 1.8 (m, 1H), 2.1 (s, 3H), |
| | | | | | | 2.1 (m, 1H), 3.3 (q, 2H), 3.4 (q, 2H), 7.11 (br, 1H), 7.5 (s, 1H), 7.99 (s, 1H), 10.9 |
| | | | | | | (br, 1H) |
| I-30 | Et | Me | OMe | Me | cis | d₆-DMSO δ = 1.1 (t, 3H), 1.4-1.9 (m, 4H), 2.0(s, 3H), 2.3-2.4 (m, 2H), 2.5 (s, |
| | | | | | | 3H), 3.4 (m, 2H), 3.5 (q, 2H), 3.6 (s, 3H), 6.78 (br, 1H), 6.88 (s, 1H), 7.57 (s, |
| | | | | | | 1H), 10.85 (br, 1H) |
| I-31 | Me | Me | OMe | Me | trans | d₆-DMSO δ = 1.4-1.9 (m, 4H), 2.0 (s, 3H), 2.5 (m, 2H), 3.2 (m 2H), 3.3 (s, 3H), |
| | | | | | | 3.6 (s, 3H), 6.78 (br, 1H), 6.88 (s, 1H), 7.78 (s, 1H), 10.55 (br, 1H) |
| I-32 | Et | Me | OMe | Me | trans | d₆-DMSO δ = 1.1 (t, 3H), 1.4-1.9 (m, 4H), 2.0(s, 3H), 2.3-2.4 (m, 2H), 2.5 (s, |
| | | | | | | 3H), 3.4 (m, 2H), 3.5 (q, 2H), 3.6 (s, 3H), 6.78 (br, 1H), 6.88 (s, 1H), 7.78 (s, |
| | | | | | | 1H), 10.85 (br, 1H) |
| I-33 | Me | Me | OMe | Me | cis | d₆-DMSO δ = 1.4-1.6 (m, 3H), 1.9 (m, 1H), 2.0 (s, 3H), 2.3-2.4 (m, 2H), 3.3 (s, |
| | | | | | | 3H), 3.4 (m, 2H), 3.5 (s, 3H), 6.78 (br, 1H), 6.98 (s, 1H), 7.59 (s, 1H), 10.5 (br, |
| | | | | | | 1H) |
| I-34 | Me | Me | F | Me | cis | d₆-DMSO δ = 1.4-1.6 (m, 3H), 1.9 (m, 1H), 2.0 (s, 3H), 2.2 (s, 3H), 2.3-2.4 (m, |
| | | | | | | 2H), 3.4 (m, 2H), 3.5 (s, 3H), 6.98 (br, 1H), 7.09 (s, 1H), 7.79 (s, 1H), 11.08 (br, |
| | | | | | | 1H) |
| I-35 | Et | Me | F | Me | cis | d₆-DMSO δ = 1.1 (t, 3H), 1.4-1.6 (m, 3H), 1.9 (m 1H), 2.0 (s, 3H), 2.2 (s, 3H), |
| | | | | | | 2.4-2.45 (m, 2H), 3.4 (m, 2H), 3.5 (q, 2H), 6.99 (br, 1H), 7.09 (s, 1H), 7.78 (s, |
| | | | | | | 1H), 11.07 (br, 1H) |
| I-36 | Me | Me | F | Me | trans | d₆-DMSO δ = 1.4-1.9 (m, 4H), 2.0 (s, 3H), 2.2 (s, 3H), 2.5 (m, 2H), 3.4 (m, 2H), |
| | | | | | | 3.5 (s, 3H), 6.98 (br, 1H), 7.09 (s, 1H), 7.79 (s, 1H), 11.05 (br, 1H) |
| I-37 | Et | Me | F | Me | trans | d₆-DMSO δ = 1.1 (t, 3H), 1.4-1.9 (m 4H), 2.0 (s, 3H), 2.2 (s, 3H), 3.4 (m, 2H), |
| | | | | | | 3.5 (q, 2H), |
| I-40 | Me | Me | F | OMe | trans | d₆-DMSO δ = 1.4-1.9 (m, 4H), 2.0 (s, 3H), 2.1-2.3 (m, 2H), 3.4 (m, 2H), 3.5 (s, |
| | | | | | | 3H), 3.7 (s, 3H), 6.78 (br, 1H), 6.82 (s, 1H), 7.84 (s, 1H) |
| I-41 | Me | Me | F | OMe | cis | d₆-DMSO δ = 1.4-1.9 (m, 4H), 2.0 (s, 3H), 2.1-2.3 (m, 2H), 3.4 (m, 2H), 3.5 (s, |
| | | | | | | 3H), 3.7 (s, 3H), 6.78 (br, 1H), 6.86 (s, 1H), 7.66 (s, 1H) |
| I-42 | Et | Me | F | OMe | cis | D₂O, NaOD δ: 0.9 (t, 3H), 1.2-1.7 (m, 4H), 1.8 (s, 3H), 2.0-2.1 (m, 2H), 3.3 (m, |
| | | | | | | 2H), 3.4 (q, 2H), 3.5 (s, 3H), 6.59 (s, 1H), 6.62 (s, 1H), 6.64 (s, 1H) |
| I-43 | Et | Me | F | OMe | trans | d6-DMSO δ = 1.1 (t, 3H), 1.4-1.9 m 4H), 2.0 (s, 3H), 2.4 (m, 1H), 3.3 (m, 2H), |
| | | | | | | 3.4 (q, 2H), 3.7 (s, 3H), 6.82 (br, 1H), 6.82 (s, 1H), 7.84 (s, 1H), 10.81 (br, 1H) |
| I-44 | allyl | Me | Cl | Me | cis | d₆-DMSO δ = 1.4-1.9 (m, 4H), 2.0 (s, 3H), 2.1 (s, 3H), 2.3 (m, 1H), 2.4 (m, 1H), |
| | | | | | | 3.4 (m, 2H), 3.95 (dt, 2H), 5.28 (dq, 2H), 5.98 (m, 1H), 7.22 (s, 1H), 7.27 (s, 1H), |
| | | | | | | 7.75 (br, 1H), 11.0 (br, 1H) |
| I-45 | allyl | Me | OMe | Me | trans | d₆-DMSO δ = 1.4-1.9 (m, 4H), 2.0 (s, 3H), 2.4 (m, 2H), 3.4 (m, 2H), 3.4 (s, 3H), |
| | | | | | | 3.6 (s, 3H), 4.0 (m, 2H), 5.2 (dt, 2H), 5.9 (m, 1H), 6.78 (br, 1H), 6.84 (s, 1H), |
| | | | | | | 7.78 (s, 1H), 10.88 (br, 1H) |
| I-46 | allyl | Me | Cl | Me | trans | d₆-DMSO δ = 1.4-1.9 (m, 4H), 2.0 (s, 3H), 2.1 (s, 3H), 2.5 (m, 2H), 3.4 (m, 2H), |
| | | | | | | 3.95 (dt, 2H), 5.28 (dq, 2H), 5.98 (m, 1H), 7.22 (s, 1H), 7.27 (s, 1H), 7.94 (br, |
| | | | | | | 1H), 11.0 (br, 1H) |
| I-47 | allyl | Me | OMe | Me | cis | d₆-DMSO δ = 1.4-1.9 (m, 3H), 2.0 (s, 3H), 2.2-2.4 (m, 2H), 3.4 (s, 3H), 3.6 (s, |
| | | | | | | 3H), 4.0 (dt, 2H), 5.28 (dq, 2H), 5.9 (m, 1H), 6.78 (s, 1H), 6.88 (s, 1H), 7.60 (s, |
| | | | | | | 1H), 10.52 (br, 1H) |
| I-50 | 2- | Me | Cl | Me | trans | d₆-DMSO δ = 1.4-1.9 (m, 4H), 2.0 (s, 3H), 2.1 (s, 3H), 2.5 (m, 2H), 3.3 (s, 3H), |
| | methoxy- | | | | | 3.4 (s, 3H), 3.4-3.5 (m, 4H), 7.22 (s, 1H), 7.27 (s, 1H), 7.94 (br, 1H) |
| | ethyl | | | | | |
| I-51 | 2- | Me | Cl | Me | cis | d6-DMSO δ = 1.4-1.9 (m, 4H), 2.0 (s, 3H), 2.1 (s, 3H), 2.3 (m 1H), 2.4 (m, 1H), |
| | methoxy- | | | | | 3.3 (s, 3H), 3.4 (s, 3H), 3.4-3.5 (m, 4H), 7.22 (s, 1H), 7.27 (s, 1H), 7.72 (br, 1H) |
| | ethyl | | | | | |
| I-52 | 2- | Me | OMe | Me | trans | d₆-DMSO δ = 1.5-1.9 (m, 4H), 2.0 (s, 3H), 3.2 (m, 2H), 3.3 (s, 3H), 3.5 (m, 4H), |
| | methoxy- | | | | | 3.7 (s, 3H), 6.78 (br, 1H), 6.79 (s, 1H), 6.88 (s, 1H), 7.78 (br, 1H), 10.5 (br, 1H) |
| | ethyl | | | | | |
| I-54 | allyl | Me | OMe | F | cis | D₂O, NaOD δ: 1.5 (m, 2H), 1.7 (m, 1H), 2.0 (m, 2H), 2.0 (s, 3H), 2.4 (m, 1H), |
| | | | | | | 2.5 (m, 1H), 3.5 (m, 2H), 3.6 (s, 3H), 4.1 (m, 2H), 5.27 (qd, 1H), 5.38 (qd, 1H), |
| | | | | | | 5.95 (m, 1H), 6.85 (s, 1H), 6.87 (s, 1H), 6.91 (s, 1H) |
| I-55 | allyl | Me | OMe | F | trans | D₂O, NaOD δ: 1.5 (m, 1H), 1.7 (m, 3H), 2.0 (m, 1H), 2.0 (s, 3H), 2.5 (m, 2H), |
| | | | | | | 3.5 (m, 2H), 3.6 (s, 3H), 4.1 (m, 2H), 5.27 (qd, 1H), 5.38 (qd, 1H), 5.95 (m, 1H), |
| | | | | | | 6.85 (s, 1H), 6.87 (s, 1H), 6.91 (s, 1H) |
| I-56 | propyl | Me | OMe | F | trans | D₂O, NaOD δ: 0.9 (t, 3H), 1.5-1.9 (m, 4H), 2.0 (s, 3H), 2.1 (m, 1H), 2.5 (m, 1H), |
| | | | | | | 3.5 (m, 4H), 3.8 (s, 3H), 6.85 (s, 1H), 6.88 (s, 1H), 6.91 (s, 1H) |
| I-57 | propyl | Me | OMe | F | cis | D₂O, NaOD δ: 0.9 (t, 3H), 1.5-1.7 (m, 4H), 1.8 (m, 1H), 1.9 (m, 1H), 2.0 (s, 3H), |
| | | | | | | 2.3 (m, 1H), 2.5 (m, 1H), 3.5 (m, 4H), 3.78 (s, 3H), 6.85 (s, 1H), 6.88 (s, 1H), |
| | | | | | | 6.91 (s, 1H) |
| I-58 | propyl | Me | OMe | Me | cis | D₂O, NaOD δ: 0.9 (t, 3H), 1.5-1.9 (m, 4H), 2.0 (s, 3H), 2.1 (s, 3H), 2.4 (m, 1H), |
| | | | | | | 2.5 (m, 1H), 3.5 (m, 4H), 3.7 (s, 3H), 6.92 (s, 1H), 7.00 (s, 1H) |
| I-59 | propyl | Me | OMe | Me | trans | D₂O, NaOD δ: 0.9 (t, 3H), 1.5-1.9 (m, 4H), 2.0 (m, 1H), 2.0 (s, 3H), 2.1 (s, 3H), |
| | | | | | | 2.5 (m, 1H), 3.5 (m, 2H), 3.7 (s, 3H), 6.92 (s, 1H), 7.0 (s, 1H) |
| I-63 | Et | Me | Br | OEt | trans | D₂O, NaOD δ: 1.2 (t, 3H), 1.3 (t, 3H), 1.5 (m, 2H), 1.7 (m, 1H), 2.0 (m, 2H), 2.0 |
| | | | | | | (s, 3H), 2.2 (m, 1H), 2.5 (m, 1H), 3.4 (d, 2H), 3.6 (q, 2H), 4.0 (q, 2H), 7.05 (s, |
| | | | | | | 1H), 7.37 (s, 1H) |
| I-64 | Et | Me | Br | OEt | cis | D₂O, NaOD δ: 1.1 (t, 3H), 1.2 (t, 3H), 1.5 (m, 2H), 1.7 (m, 1H), 2.0 (m, 2H), 2.0 |
| | | | | | | (s, 3H), 2.4 (m, 1H), 2.5 (m, 1H), 3.4 (d, 2H), 3.6 (q, 2H), 4.0 (q, 2H), 7.05 (s, |
| | | | | | | 1H), 7.37 (s, 1H) |
| I-66 | Me | Me | Br | OEt | cis | D₂O, NaOD δ: 1.2 (t, 3H), 1.5 (m, 2H), 1.7 (m, 1H), 1.9 (m, 2H), 2.0 (s, 3H), 2.4 |
| | | | | | | (m, 1H), 2.5 (m, 1H), 3.4 (s, 3H), 3.5 (d, 2H), 4.0 (q, 2H), 7.05 (s, 1H), 7.37 (s, |
| | | | | | | 1H) |
| I-67 | allyl | Me | Me | Br | trans | d₆-DMSO δ = 1.5-1.9 (m, 4H), 2.0 (s, 3H), 2.1 (s, 3H), 3.4 (m, 4H), 4.0 (m, 2H), |
| | | | | | | 5.22 (qd, 1H), 5.38 (qd, 1H), 5.96 (m, 1H), 7.25 (s, 1H), 7.44 (s, 1H), 7.93 (br, |
| | | | | | | 1H) |
| I-68 | propyl | Me | Cl | Me | cis | d₆-DMSO δ = 0.9 (t, 3H), 1.5-1.9 (m, 4H), 2.0 (s, 3H), 2.1 (s, 3H), 2.4 (m, 2H), |
| | | | | | | 3.4 (m, 4H), 7.22 (s, 1H), 7.27(s, 1H), 7.73 (br, 1H) |
| I-69 | Me | Me | Br | OEt | trans | D₂O, NaOD δ: 1.3 (t, 3H), 1.5 (m, 1H), 1.6-1.9 (m, 3H), 2.0 (s, 3H), 2.1 (m, 1H), |
| | | | | | | 2.5 (m, 1H), 3.4 (s, 3H), 3.5 (m, 2H), 4.0 (q, 2H), 7.05 (s, 1H), 7.37 (s, 1H) |
| I-70 | allyl | Me | Me | Br | cis | d₆-DMSO δ = 1.5-1.9 (m, 4H), 2.0 (s, 3H), 2.1 (s, 3H), 2.3-2.5 (m, 2H), 3.4 (m, |
| | | | | | | 4H), 4.0 (m, 2H), 5.22 (qd, 1H), 5.38 (qd, 1H), 5.95 (m, 1H), 7.25 (s, 1H), 7.44 |
| | | | | | | (s, 1H), 7.74 (br, 1H) |
| I-71 | propyl | Me | Cl | Me | trans | d₆-DMSO δ = 0.9 (t, 3H), 1.5-1.9 (m, 4H), 2.0 (s, 3H), 2.1 (s, 3H), 3.4 (m, 4H), |
| | | | | | | 7.22 (s, 1H), 7.27 (s, 1H), 7.93 (br, 1H) |
| I-72 | Me | Me | Cl | OMe | trans | D₂O, NaOD δ: 1.5-1.9 (m, 3H), 2.0 (s, 3H), 2.1 (m, 1H), 2.4 (m, 1H), 2.5 (m, |
| | | | | | | 1H), 3.4 (s, 3H), 3.5 (m, 2H), 3.7 (s, 3H), 7.01 (s, 1H), 7.18 (s, 1H) |
| I-73 | Me | Me | Cl | OMe | cis | D₂O, NaOD δ: 1.5-1.9 (m, 4H), 2.0 (s, 3H), 2.4 (m, 1H), 2.5 (m, 1H), 3.4 (s, 3H), |
| | | | | | | 3.5 (m, 2H), 3.7 (s, 3H), 7.01 (s, 1H), 7.18 (s, 1H) |
| I-74 | prop-2- | Me | F | OMe | cis | D₂O, NaOD δ: 1.5 (m, 2H), 1.6 (m, 1H), 2.0 (m, 2H), 2.1 (s, 3H), 2.4 (m, 1H), |
| | yn-1-yl | | | | | 2.5 (m, 1H), 3.6 (d, 2H), 3.7 (s, 3H), 4.2 (s, 2H), 6.85 (s, 1H), 6.88 (s, 1H), 6.91 |
| | | | | | | (s, 1H) |
| I-75 | prop-2- | Me | F | OMe | trans | D₂O, NaOD δ: 1.5 (m, 1H), 1.6-1.7 (m, 3H), 2.0 (m, 1H), 2.1 (s, 3H), 2.5 (m, |
| | yn-1-yl | | | | | 1H), 3.6 (d, 2H), 3.7 (s, 3H), 4.3 (s, 2H), 6.85 (s, 1H), 6.88 (s, 1H), 6.91 (s, 1H) |
| I-76 | prop-2- | Me | Me | OMe | cis | D₂O, NaOD δ: 1.5 (m, 2H), 1.7 (m, 1H), 2.0 (m, 2H), 2.1 (s, 3H), 2.2 (s, 3H), 2.4, |
| | yn-1-yl | | | | | (m, 1H), 2.5 (m, 1H), 3.6 (d, 2H), 3.7 (s, 3H), 4.2 (s, 2H), 6.92 (s, 1H), 7.00 (s, |
| | | | | | | 1H) |
| I-77 | prop-2- | Me | Me | OMe | trans | D₂O, NaOD δ: 1.5 (m, 2H), 1.6-1.9 (m, 2H), 2.0 (s, 3H), 2.1 (m, 1H), 2.2 (s, 3H), |
| | yn-1-yl | | | | | 2.5 (m, 1H), 3.6 (m, 2H), 3.7 (s, 3H), 4.2 (s, 2H), 6.90 (s, 1H), 7.00 (s, 1H) |
| I-78 | prop-2- | Me | Cl | Me | trans | d₆-DMSO δ = 1.4-1.9 (m, 5H), 2.0 (s, 3H), 2.1 (s, 3H), 3.2 (s, 3H), 3.5 (m, 2H), |
| | yn-1-yl | | | | | 4.0 (s, 1H), 7.22 (s, 1H), 7.27 (s, 1H), 7.94 (br, 1H) |
| I-79 | prop-2- | Me | Cl | Me | cis | d₆-DMSO δ = 1.4-1.9 (m, 4H), 2.0 (s, 3H), 2.1 (s, 3H), 2.3-2.4 (m, 2H), 3.2 (s, |
| | yn-1-yl | | | | | 3H), 3.5 (m, 2H), 4.0 (s, 1H), 7.22 (s, 1H), 7.27 (s, 1H), 7.77 (br, 1H) |
| I-81 | Me | Me | OMe | Br | trans | d₆-DMSO δ = 1.4-1.9 (m, 4H), 2.0 (s, 3H), 2.4 (m, 2H), 3.2 (s, 3H), 3.3 (m, 2H), |
| | | | | | | 3.7 (s, 3H), 7.0 (s, 1H), 7.2 (s, 1H), 7.8 (br, 1H) |

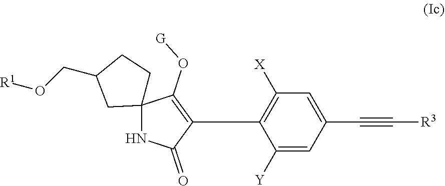

TABLE 2
Compounds of the general formula (Ic)

| Example | | | | | | Stereo- | |
|---|---|---|---|---|---|---|---|
| No. | G | R¹ | R³ | X | Y | chemistry | ¹H NMR (400 MHz, CDCl₃ δ in ppm) |
| P-01 | —CO₂Et | Et | Me | OMe | Br | trans | 1.17 (t, 3H), 1.21 (t, 3H), 1.61-1.68 (m, 1H), 1.88-2.03 (m, 4H), 2.05 (s, 3H), |
| | | | | | | | 2.22-2.30 (m, 1H), 2.33-2.51 (m 1H), 3.38 (m, 2H), 3.49 (q, 2H), 3.78 (s, |
| | | | | | | | 3H), 4.05 (q, 2H), 6.87 (s, 1H), 7.25 (s, 1H) |
| P-02 | —CO₂Et | Me | H | OMe | Cl | cis | 1.18 (t, 3H), 1.71-1.87 (m, 3H), 1.98-2.18 (m 2H), 2.41-2.52 (m 2H), 3.35- |
| | | | | | | | 3.45 (m 2H), 3.40 (s, 3H), 3.81 (s, 3H), 4.08 (q, 2H), 6.92 (s, 1H), 7.17 (s, |
| | | | | | | | 1H) |
| P-03 | —COiPr | Et | Me | Me | Cl | trans | 1.02-1.12 (d, 6H), 1.16-1.21 (t, 3H), 1.56-1.64 (m, 1H), 1.76-2.00 (m, 4H), |
| | | | | | | | 2.03 (s, 3H), 2.05-2.10 (m, 1H), 2.27 (s, 3H), 2.46-2.53 (m, 1H), 2.57-2.66 |
| | | | | | | | (hept, 1H), 3.33-3.40 (m, 2H), 3.44-3.51 (q, 2H), 6.76 (br, NH), 7.15 (s, |
| | | | | | | | 1H), 7.24 (s, 1H) |
| P-04 | —COiPr | Me | Me | OMe | Br | cis | 1.17 (d, 6H), 1.66-1.80 (m, 2H), 1.81-2.10 (m, 2H), 2.04 (s, 3H), 2.26-2.49 |
| | | | | | | | (m, 2H), 2.58-2.67 (m, 1H), 3.34-3.47 (m, 2H), 3.39 (s, 3H), 3.78 (s, 3H), |
| | | | | | | | 6.85 (br, 1H), 7.19 (s, 1H), 7.22 (s, 1H) |
| P-05 | —CO₂Et | Et | Me | OMe | Br | cis | 1.17 (t, 3H), 1.29 (t, 3H), 1.68-1.73 (m, 1H), 1.83-1.90 (m, 2H), 1.98-2.13 |
| | | | | | | | (m, 2H), 2.04 (s, 3H), 2.38-2.53 (m, 2H), 3.39-3.44 (m, 2H), 3.51 (q, 2H), |
| | | | | | | | 3.77 (s, 3H), 4.04 (q, 2H), 6.88 (br, NH, 7.23 (s, 1H), 7.25 (s, 1H) |
| P-06 | —CO₂Et | Me | Me | Et | Cl | trans | 1.15 (t, 3H), 1.18 (t, 3H), 1.59-1.85 (m, 1H), 1.83-2.00 (m, 4H), 2.04 (s, 3H), |
| | | | | | | | 2.21-2.59 (m, 3H), 3.30-3.38 (m, 2H), 3.34 (s, 3H), 4.04 (q, 2H), 7.20 (s, |
| | | | | | | | 1H), 7.29 (s, 1H) |
| P-07 | —COiPr | Et | Me | Et | Cl | trans | 1.04 (d, 6H), 1.18 (t, 3H), 1.20 (t, 3H), 1.62-1.64 (m, 1H), 1.79-2.03 (m, 3H), |
| | | | | | | | 2.04 (s, 3H), 2.09-2.26 (m, 1H), 2.47-2.53 (m 2H), 2.59 (q, 2H), 3.33-3.39 |
| | | | | | | | (m, 2H), 3.38 (q, 2H), 6.38 (br, NH), 7.20 (s, 1H), 7.26 (s, 1H) |
| P-08 | —CO₂Et | Me | Me | OMe | Br | trans | 1.17 (t, 3H), 1.64-1.85 (m, 1H), 1.85-1.88 (m, 3H), 2.05 (s, 3H), 2.24-2.30 |
| | | | | | | | (m, 1H), 2.46-2.51 (m, 1H), 3.35 (s, 3H), 3.78 (s, 3H), 4.05 (q, 2H), 6.13 (br, |
| | | | | | | | NH), 6.87 (s, 1H), 7.25 (s, 3H) |
| P-09 | —CO₂Et | Et | Me | Me | Cl | trans | 1.13 (t, 3H), 1.20 (t, 3H), 1.85 (m, 1H), 1.90 (m, 3H), 2.03 (s, 3H), 2.28 (m, |
| | | | | | | | 1H), 2.51 (m, 1H), 3.38 (m, 2H), 3.49 (q, 2H), 4.05 (q, 2H), 7.16 (s, 1H), |
| | | | | | | | 7.27 (s, 1H) |
| P-11 | —CO₂Et | Et | Me | Me | Cl | cis | 1.17 (t, 3H), 1.28 (t, 3H), 1.67-1.73 (m 1H), 1.79-1.91 (m 2H), 197-2.20 (m, |
| | | | | | | | 2H), 2.04 (s, 3H), 2.26 (s, 3H), 2.35-2.44 (m, 1H), 2.50 (m, 1H), 3.39-3.52 |
| | | | | | | | (m, 2H), 3.58 (q, 2H), 4.06 (q, 2H), 7.15 (s, 1H), 7.27 (s, 1H) |
| P-12 | —COiPr | Et | Me | Et | Cl | cis | 1.21 (t, 3H), 1.30 (t, 3H), 1.70 (m, 1H), 1.83-1.99 (m, 3H), 2.04 (s, 3H), 2.27 |
| | | | | | | | (m, 1H). 2.55-2.67 (m, 4H), 3.40-3.60 (m, 4H), 7.19 (s, 1H), 7.25 (s, 1H) |
| P-13 | —CO₂Et | Me | Me | OMe | Br | cis | 1.17 (t, 3H), 1.69-1.89 (m, 3H), 1.97-2.16 (m, 2H), 2.04 (s, 3H), 2.37-2.53 |
| | | | | | | | (m, 2H), 3.34-3.44 (m, 2H), 3.40 (s, 3H), 3.78 (s, 3H), 4.05 (q, 2H), 6.81 (br, |
| | | | | | | | NH), 6.86 (s, 1H), 7.25 (s, 1H) |
| P-14 | —COiPr | Me | Me | Et | Cl | trans | 1.15 (t, 3H), 1.59-1.65 (m 1H), 1.83-2.00 (m 3H), 2.03 (s, 3H), 2.20.2.32 (m, |
| | | | | | | | 1H), 2.25 (s, 3H), 2.48-2.56 (m, 1H), 3.30-3.39 (m, 2H), 3.34 (s, 3H), 4.06 |
| | | | | | | | (q, 2H), 6.64 (br, NH), 7.16 (s, 1H), 7.27 (s, 1H) |
| P-15 | —CO₂Et | Me | Me | Et | Cl | cis | 1.13 (t, 3H), 1.19 (t, 3H), 1.67-1.71 (m, 1H), 1.79-1.93 (m, 2H), 2.04 (s, 3H), |
| | | | | | | | 2.09-2.20 (m, 2H), 2.36-2.87 (m, 2H), 3.34 (m, 1H), 3.41 (s, 3H), 3.42-3.48 |
| | | | | | | | (m, 1H), 4.04 (q, 2H), 7.20 (s, 1H), 7.28 (s, 1H) |
| P-16 | —COiPr | Me | Me | Me | Cl | cis | 1.03 (d, 6H), 1.72 (m, 1H), 1.82-2.00 (m, 3H), 2.03 (s, 3H), 2.08-2.25 (m, |
| | | | | | | | 1H), 2.27 (s, 3H), 2.39-2.50 (m, 2H), 2.61 (m, 1H), 3.36 (m, 1H), 3.41 (s, |
| | | | | | | | 3H), 3.45 (m, 1H), 7.14 (s, 1H), 7.24 (s, 1H) |
| P-17 | —COiPr | Et | Me | Me | Cl | cis | 1.03-1.08 (d, 6H), 1.28-1.30 (t, 3H), 1.66-1.73 (m, 1H), 1.78-2.01 (m, 3H), |
| | | | | | | | 2.03 (s, 3H), 2.27 (s, 3H), 2.42 (m, 1H), 2.60-2.83 (hept, 1H), 3.39 (m, 2H), |
| | | | | | | | 3.54-3.61 (q, 2H), 7.14 (s, 1H), 7.24 (s, 1H) |
| P-18 | —CO₂Et | Me | Me | Me | Cl | trans | 1.15 (t, 3H), 1.59-1.65 (m, 1H), 1.80-2.00 (m, 4H), 2.03 (s, 3H), 2.20-2.30 |
| | | | | | | | (m, 1H), 2.25 (s, 3H), 2.48-2.56 (m, 1H), 3.30-3.39 (m, 2H), 3.34 (s, 3H), |
| | | | | | | | 4.06 (q, 2H), 6.64 (br, Nh), 7.16 (s, 1H), 7.27 (s, 1H) |
| P-19 | —CO₂Et | Me | H | OMe | Cl | trans | 1.18 (t, 3H), 1.63-1.84 (m, 1H), 1.88-2.01 (m, 4H), 2.24-2.28 (m, 1H), 2.27- |
| | | | | | | | 2.50 (m, 1H), 3.32-3.37 (m, 2H), 3.35 (s, 3H), 3.80 (s, 3H), 4.06 (q, 2H), |
| | | | | | | | 6.19 (br, NH), 6.92 (s, 1H), 7.17 (s, 1H) |
| P-20 | —CO₂Et | Et | Me | Et | Cl | cis | 1.13 (t, 3H), 1.21 (t, 3H), 1.30 (t, 3H), 1.70 (m, 1H) 1.85-1.92 (m, 2H), 2.04 |
| | | | | | | | (s, 3H), 2.13 (m, 1H), 2.20-2.63 (m, 1H), 3.39 (q, 2H), 3.58 (q, 2H), 4.04 (q, |
| | | | | | | | 2H), 7.20 (s, 1H), 7.29 (s, 1H) |
| P-21 | —CO₂Et | Et | Me | Et | Cl | trans | 1.13 (t, 3H), 1.17 (t, 3H), 1.21 (t, 3H), 1.65 (m, 1H), 1.85-1.99 (m, 3H), 2.04 |
| | | | | | | | (s, 3H), 2.28 (m, 1H), 2.50 (m, 1H), 2.53 (q, 2H), 3.41 (q, 2H), 3.51 (q, 2H), |
| | | | | | | | 4.06 (q, 2H), 7.2 (s, 1H), 7.29 (s, 1H) |
| P-22 | —COiPr | Me | Me | Et | Cl | cis | 1.13-1.18 (d, 6H), 1.64-1.68 (m, 1H), 1.79-2.00 (m, 3 H), 2.04 (s, 3H), 2.21- |
| | | | | | | | 2.48 (m, 1H), 2.55-.265 (m, 3H), 3.30-3.39 (m, 2H), 3.34 (s, 3H), 4.04 (q, |
| | | | | | | | 2H), 6.63 (br, NH), 7.20 (s, 1H), 7.29 (s, 1H) |
| P-23 | —COiPr | Et | Me | OMe | Br | trans | 1.17 (d, 6H), 1.19 (t, 3H), 1.58-1.63 (m 1H), 1.79-2.00 (m, 4H), 2.04 (s, 3H), |
| | | | | | | | 2.11-2.23 (m, 1H), 2.46-2.52 (m, 1H), 2.63 (hept, 1H), 3.36-3.39 (m, 2H), |
| | | | | | | | 3.47 (q, 2H), 3.87 (s, 3H), 6.38 (br, NH), 6.86 (s, 1H), 7.22 (s, 1H) |
| P-24 | —CO₂Et | Me | Me | Me | Cl | cis | 1.12-1.17 (t, 3H), 1.68-1.78 (m, 1H), 1.80-1.87 (m, 2H), 2.03 (s, 3H), 2.16 |
| | | | | | | | (m, 2H), 2.28 (s, 3H), 2.34-2.54 (m, 2H), 3.34-3.88 (m, 2H), 3.41 (s, 3H), |
| | | | | | | | 4.04 (q, 2H), 7.15 (s, 1H), 7.28 (s, 1H) |
| P-25 | —COiPr | Et | Me | OMe | Cl | trans | 1.09 (d, 6H), 1.19 (t, 3H), 1.60-163 (m, 1H), 1.80-2.00 (m, 4H), 2.04 (s, 3H), |
| | | | | | | | 2.13-2.27 (m, 1H), 2.42-2.50 (m, 1H), 2.62 (hept, 1H), 3.37 (m, 2H), 3.48 (q, |
| | | | | | | | 2H), 3.71-3.82 (m, 1H), 3.78 (s, 3H), 6.13 (br, NH), 6.82 (s, 1H), 7.04 (s, |
| | | | | | | | 1H) |
| P-26 | —CO₂Et | Et | Me | OMe | Cl | trans | 1.15 (t, 3H), 1.19 (t, 3H), 1.62-1.64 (m 1H), 1.80-2.00 (m, 4H), 2.05 (s, 3H), |
| | | | | | | | 2.21-2.29 (m, 1H), 2.30-2.51 (m, 1H), 3.37 (m, 2H), 3.49 (q, 2H), 3.78 (s, |
| | | | | | | | 3H), 4.06 (q, 2H), 6.19 (br, NH), 6.83 (s, 1H), 7.07 (s, 1H) |
| P-27 | —CO₂Et | Et | Me | OMe | Cl | trans | 1.18 (t, 6H), 1.66-1.84 (m, 1H), 1.92-2.03 (m, 4H), 2.25-2.29 (m 1H), 2.40- |
| | | | | | | | 2.52 (m, 1H), 3.36 (m, 2H), 3.48 (q, 2H), 3.80 (s, 3H), 4.08 (q, 2H), 6.11 (br, |
| | | | | | | | NH), 6.93 (s, 1H), 7.18 (s, 1H) |
| P-28 | —CO₂Et | Et | Me | Me | Cl | cis | 1.2 (t, 3H), 1.4 (m, 2H), 1.6-1.9 (m, 2H), 2.01 (s, 3H), 2.4 (s, 3H), 2.5 (m, |
| | | | | | | | 3H), 3.4-3.5 (m, 2H), 3.6 (q, 2H), 4.0 (q, 2H), 6.2 (br, 1H), 7.15 (s, 1H), 7.25 |
| | | | | | | | (s, 1H) |
| P-29 | —COiPr | Et | Me | Me | Cl | cis | 1.18 (d, 6H), 1.45 (t, 2H), 1.6-1.9 (m, 3H), 2.0 (s, 3H), 2.1 (m, 1H), 2.3 (s, |
| | | | | | | | 3H), 2.4 (m, 1H), 2.6 (hept, 1H), 3.4-3.5 (q, 2H), 3.6 (m, 2H), 6.5 (br, 1H), |
| | | | | | | | 7.22 (s, 1H), 7.14 (s, 1H) |
| P-30 | —CO2Et | Me | Me | Me | Br | trans | 1.18 (t, 3H), 1.6-1.9 (m, 3H), 2.0 (s, 3H), 2.5 (m, 1H), 3.40 (m, 5H), 4.1 (q, |
| | | | | | | | 2H), 6.56 (br, 1H), 7.19 (s, 1H), 7.45 (s, 1H) |
| P-31 | —CO2Et | Me | Me | Me | Br | cis | 1.2 (t, 3H), 1.58-1.9 (m, 3H), 2.0 (s, 3H), 2.5 (m, 2H), 3.4 (m, 5H), 4.08 (q, |
| | | | | | | | 2H), 6.89 (br, 1H), 7.19 (s, 1H), 7.45 (s, 1H) |
| P-32 | —COiPr | Me | Me | Me | Br | cis | 1.1 (d, 6H), 1.57-1.9 (m, 3H), 2.01 (s, 3H), 2.2 (s, 3H), 2.5 (m, 1H), 2.7 |
| | | | | | | | (hept, 1H), 3.3 |
| | | | | | | | (s, 3H), 3.4 (m, 2H), 6.83 (br, 1H), 7.18 (s, 1H), 7.43 (s, 1H) |
| P-33 | —COiPr | Me | Me | Me | Br | trans | 1.1 (d, 6H), 1.57-1.9 (m, 3H), 2.01 (s, 3H), 2.2 (s, 3H), 2.5 (m, 1H), 2.6 |
| | | | | | | | (hept, 1H), 3.4 (s, 3H), 3.5 (m, 2H), 6.37 (br, 1H), 7.19 (s, 1H), 7.43 (s, 1H) |
| P-34 | —CO2Et | Et | Me | Me | Br | trans | 1.1 (t, 3H), 1.2 (t, 3H), 1-6-1.9 (m 3H), 2.0 (s, 3H), 2.1 (m, 1H), 2.3 (s, 3H), |
| | | | | | | | 2.5 (m, 2H), 3.4 (m, 2H), 3.6 (q, 2H), 4.1 (q, 2H), 6.24 (br, 1H), 7.20 (s, 1H), |
| | | | | | | | 7.46 (s, 1H) |
| P-35 | —CO2Et | Et | Me | Me | Br | cis | 1.1 (t, 3H), 1.3 (t, 3H), 1-6-1.9 (m 3H), 2.0 (s, 3H), 2.1 (m, 1H), 2.4 (s, 3H), |
| | | | | | | | 2.5 (m, 2H), 3.4 (m, 2H), 3.6 (q, 2H), 4.1 (q, 2H), 7.02 (br, 1H), 7.19 (s, 1H), |
| | | | | | | | 7.46 (s, 1H) |
| P-36 | —COiPr | Et | Me | Me | Br | trans | 1.1 (d, 6H), 1.3 (t, 3H), 1.6-1.9 (m, 3H), 2.0 (s, 3H), 2.1 (m 1H), 2.4 (s, 3H), |
| | | | | | | | 2.5 (m, 1H), 2.6 (hept, 1H), 3.4 (m, 2H), 3.6 (q, 2H), 6.49 (br, 1H), 7.18 (s, |
| | | | | | | | 1H), 7.43 (s, 1H) |
| P-37 | —COiPr | Et | Me | Me | Br | cis | 1.1 (d, 6H), 1.3 (t, 3H), 1.6-1.9 (m, 4H), 2.0 (s, 3H), 2.1 (m 1H), 2.4 (s, 3H), |
| | | | | | | | 2.5 (m, 1H), 2.6 (hept, 1H), 3.4 (m, 2H), 3.6 (q, 2H), 6.98 (br, 1H), 7.18 (s, |
| | | | | | | | 1H), 7.43 (s, 1H) |
| P-38 | —CO2Et | Et | Me | Et | Br | cis | 1.1 (t, 6H), 1.2 (t, 3H), 1.6-1.9 (m, 4H), 2.1 (s, 3H), 2.4 (m, 1H), 2.5 (m, |
| | | | | | | | 3H), 3.4 (q, 2H), 3.6 (q, 2H), 4.1 (q, 2H), 7.0 (br, 1H), 7.2 (s, 1H), 7.46 (s, 1H) |
| P-39 | —CO2Et | Me | Me | Et | Br | cis | 1.2 (t, 6H), 1.6-1.9 (m, 3H), 2.0 (s, 3H), 2.4 (m, 1H), 2.5 (m, 3H), 3.3 (m, |
| | | | | | | | 2H), 3.4 (s, 3H), 4.1 (q, 2H), 6.8 (br, 1H), 7.22 (s, 1H), 7.46 (s, 1H) |
| P-40 | —CO2Et | Me | Me | Et | Br | trans | 1.1 (t, 6H), 1-6-1.9 (m, 3H), 2.1 (s, 3H), 2.2 (m, 1H), 2.6 (m, 3H), 3.3 (q, |
| | | | | | | | 2H), 3.3 (s, 3H), 4.1 (q, 2H), 6.1 (br, 1H), 7.2 (s, 1H), 7.4 (s, 1H) |
| P-41 | —COiPr | Et | Me | Et | Br | trans | 1.1 (d, 6H), 1.2 (t, 6H), 1.6-1.9 (m, 4H), 2.0 (s, 3H), 2.1 (m, 1H), 2.4 (hept, |
| | | | | | | | 1H), 2.6 (m, 3H), 3.4 (q, 2H), 3.5 (q, 2H), 6.25 (br, 1H), 7.15 (s, 1H), 7.45 |
| | | | | | | | (s, 1H) |
| P-42 | —CO2Et | Et | Me | Et | Br | trans | 1.1 (t, 9H), 1.6-1.9 (m, 4H), 2.1 (s, 3H), 2.4 (m, 1H), 2.5 (m, 3 H), 3.4 (q, |
| | | | | | | | 2H), 3.6 (q, 2H), 4.1 (q, 2H), 6.42 (br, 1H), 7.2 (s, 1H), 7.46 (s, 1H) |
| P-43 | —COiPr | Et | Me | Et | Br | cis | 1.1 (d, 6H), 1.2 (t, 6H), 1.3 (t, 3H), 1.6-1.9 (m, 3H), 2.0 (s, 3H), 2.2-2.5 (m, |
| | | | | | | | 1H), 2.6 (m 2H), 3.4 (q, 2H), 3.6 (q, 2H), 6.8 (br, 1H), 7.15 (s, 1H), 7.44 (s, |
| | | | | | | | 1H) |
| P-44 | —COiPr | Me | Me | Et | Br | trans | 1.1 (d, 6H), 1.2 (t, 3H), 1.6-1.9 (m, 4H), 2.1 (s, 3H), 2.3 (m, 1H), 2.63 (m, |
| | | | | | | | 4H), 3.4 (m, 2H) 3.34 (s, 3H), 6.24 (br, 1H), 7.15 (s, 1H), 7.45 (s, 1H) |
| P-45 | —COiPr | Me | Me | Et | Br | cis | 1.1 (d, 6H), 1.2 (t, 3H), 1.4-1.9 (m, 4H), 2.1 (s, 3H), 2.3 (m, 1H), 2.63 (m, |
| | | | | | | | 4H), 3.4 (m, 2H) 3.5 (s, 3H), 6.8 (br, 1H), 7.15 (s, 1H), 7.45 (s, 1H) |
| P-46 | —COiPr | Me | Me | OMe | Me | cis | 1.0 (d, 6H), 1.6-1.9 (m, 4H), 2.1 (s, 3H), 2.2 (s, 3H), 2.3-2.6 (m, 3H), 3.4 (m, |
| | | | | | | | 1H), 3.4 (s, 3H), 3.7 (s, 3H), 6.6 (br, 1H), 6.7 (s, 1H), 6.9 (s, 1H) |
| P-47 | —CO2Et | Me | Me | OMe | Me | trans | 1.1 (t, 3H), 1.6-1.9 (m, 4H), 2.0 (s, 3H), 2.2 (s, 3H), 2.3 (m, 1H), 2.6 (m, |
| | | | | | | | 1H), 3.4 (q, 2H), 3.4 (s, 3H), 3.7 (s, 3H), 4.0 (q, 2H), 6.39 (br, 1H), 6.75 (s, |
| | | | | | | | 1H), 6.89 (s, 1H) |
| P-48 | —CO2Et | Et | Me | OMe | Me | trans | 1.1 (t, 3H), 1.2 (t, 3H), 1.5-1.9 (m, 3H), 2.0 (s, 3H), 2.2 (s, 3H), 2.4 (m, 1H), |
| | | | | | | | 2.6 (m , 1H), 3.4 (m, 2H), 3.5 (q, 2H), 3.6 (s, 3H), 4.0 (q, 2H), 6.30 (br, 1H), |
| | | | | | | | 6.75 (s, 1H), 6.89 (s, 1H) |
| P-49 | —CO2Et | Et | Me | OMe | Me | cis | 1.1 (t, 3H), 1.2 (t, 3H), 1.6-1.9 (m, 3H), 2.0 (s, 3H), 2.2 (s, 3H), 2.4-2.6 (m, |
| | | | | | | | 2H), 3.4 (m, 2H), 3.5 (q, 2H), 3.6 (s, 3H), 4.0 (q, 2H), 6.74 (br, 1H), 6.81 (s, |
| | | | | | | | 1H), 6.89 (s, 1H) |
| P-50 | —COiPr | Et | Me | OMe | Me | trans | 1.1 (d, 6H), 1.2 (t, 3H), 1.6-1.9 (m, 3H), 2.0 (s, 3H), 2.2 (s, 3H), 2.4 (m, 1H), |
| | | | | | | | 2.5 (hept, 1H), 3.4 (m, 2H), 3.5 (q, 2H), 3.7 (s, 3H), 6.14 (br, 1H), 6.73 (s, |
| | | | | | | | 1H), 6.88 (s, 1H) |
| P-51 | —CO2Et | Me | Me | F | Me | cis | 1.1 (t, 3H), 1.6-1.7 (m, 3H), 2.0 (s, 3H), 2.1 (m, 1H), 2.2 (s, 3H), 2.4-2.5 (m, |
| | | | | | | | 2H), 3.4 (m, 2H), 3.5 (s, 3H), 4.0 (q, 2H), 6.84 (br, 1H), 6.92 (s, 1H), 7.06 (s, |
| | | | | | | | 1H) |
| P-52 | —CO2Et | Me | Me | F | Me | trans | 1.1 (t, 3H), 1.5-1.9 (m, 4H), 2.0 (s, 3H), 2.1 (m, 1H), 2.2 (s, 3H), 2.4-2.5 (m, |
| | | | | | | | 2H), 3.4 (m, 2H), 3.5 (s, 3H), 4.0 (q, 2H), 6.43 (br, 1H), 6.92 (s, 1H), 7.06 (s, |
| | | | | | | | 1H) |
| P-53 | —CO2Et | Et | Me | F | Me | cis | 1.1 (t, 3H), 1.2 (t, 3H), 1.6-1.9 (m, 4H), 2.0 (s, 3H), 2.2 (s, 3H), 2.4-2.5 (m, |
| | | | | | | | 2H), 3.4 (m, 2H), 3.5 (q, 2H), 4.0 (q, 2H), 6.91 (br, 1H), 6.92 (s, 1H), 7.07 (s, |
| | | | | | | | 1H) |
| P-54 | —CO2Et | Et | Me | F | Me | trans | 1.1 (t, 3H), 1.15 (t, 3H), 1.6 (m, 1H), 1.6-1.9 (m, 3H), 2.0 (s, 3H), 2.3-2.4 (m, |
| | | | | | | | 1H), 2.2 (s, 3H), 2.5 (m, 1H), 3.4 (m, 2H), 3.5 (q, 2H), 4.0 (q, 2H), 6.54 (br, |
| | | | | | | | 1H), 6.92 (s, 1H), 7.06 (s, 1H) |
| P-55 | —CO2Et | Me | Me | OMe | Me | cis | 1.1 (t, 3H), 1.6-1.9 (m 3H), 2.0 (s, 3H), 2.4-2.6 (m 2H), 3.35 (m, 2H), 3.4 (s, |
| | | | | | | | 3H), 3.7 (s, 3H), 4.0 (q, 2H), 6.71 (br, 1H), 6.77 (s, 1H), 6.89 (s, 1H) |
| P-56 | —COiPr | Et | Me | Me | F | trans | 1.1 (d, 6H), 1.3 (t, 3H), 1.6-1.9 (m, 5H), 2.0 (s, 3H), 2.4 (m, 1H), 2.5 (hept, |
| | | | | | | | 1H), 3.4 (m, 2H), 3.5 (q, 2H), 6.18 (br, 1H), 6.98 (s, 1H), 7.04 (s, 1H) |
| P-57 | —COiPr | Me | Me | Me | F | trans | 1.1 (d, 6H), 1.6-2 (m, 5H), 2.0 (s, 3H), 2.2 (s, 3H), 2.4 (m, 1H), 2.5 (hept, |
| | | | | | | | 1H), 3.4 (m, 2H), 3.4 (s, 3H), 6.33 (br, 1H), 6.90 (s, 1H), 7.05 (s, 1H) |
| P-58 | —COiPr | Me | Me | Me | F | cis | 1.1 (d, 6H), 1.6-1.9 (m, 3H), 2.0 (s, 3H), 2.2 (s, 3H), 2.4 (m, 2H), 2.5 (hept, |
| | | | | | | | 1H), 3.4 (m, 2H), 3.5 (s, 3H), 6.86 (br, 1H), 6.90 (s, 1H), 7.05 (s, 1H) |
| P-59 | —COiPr | Et | Me | Me | F | cis | 1.1 (d, 6H), 1.3 (t, 3H), 1.6-1.9 (m, 4H), 2.0 (s, 3H), 2.4 (m, 2H), 2.5 (hept, |
| | | | | | | | 1H), 3.4 (m, 2H), 3.5 (q, 2H), 6.87 (br, 1H), 6.90 (s, 1H), 7.04 (s, 1H) |
| P-60 | —COiPr | Et | Me | OMe | Me | cis | 1.1 (d 6H), 1.6-1.9 (m, 3H), 2.0 (s, 3H), 2.2 (s, 3H), 2.3-2.6 (m, 2H), 3.4 (m, |
| | | | | | | | 2H), 3.6 (q, 2H), 3.7 (s, 3H), 6.73 (br, 1H), 6.95 (s, 1H), 7.27 (s, 1H) |
| P-61 | —CO2Et | Et | Me | F | OMe | trans | 1.1 (t, 3H), 1.2 (t, 3H), 1.6-1.9 (m, 4H), 2.0 (s, 3H), 2.2 (m, 1H), 2.5 (m, 1H), |
| | | | | | | | 3.4 (m, 2H), 3.5 (q, 2H), 3.7 (s, 3H), 4.0 (q, 2H), 6.10 (br, 1H), 6.71 (s, 1H), |
| | | | | | | | 6.79 (s, 1H) |
| P-62 | —CO2Et | Me | Me | F | OMe | trans | 1.2 (t, 3H), 1.6-1.9 (m, 4H), 2.0 (s, 3H), 2.4-2.5 (m, 2H), 3.4 (m, 2H), 3.4 (s, |
| | | | | | | | 3H), 3.6 (s, 3H), 4.0 (q, 2H), 6.07 (br, 1H), 6.72 (s, 1H), 6.79 (s, 1H) |
| P-63 | —CO2Et | Me | Me | F | OMe | cis | 1.2 (t, 3H), 1.6-1.9 (m, 3H), 2.0 (s, 3H), 2.1 (m, 1H), 2.4-2.5 (m, 2H), 3.4 (m, |
| | | | | | | | 2H), 3.4 (s, 3H), 3.6 (s, 3H), 4.0 (q, 2H), 6.70 (br, 1H), 6.72 (s, 1H), 6.73 (s, |
| | | | | | | | 1H) |
| P-64 | —COiPr | Et | Me | OMe | F | trans | 1.1 (d, &h), 1.2 (t, 3H), 1.6-1.9 (m, 4H), 2.0 (s, 3H), 2.2 (m, 1H), 2.4 (m 1H), |
| | | | | | | | 2.6 (hept, 1H), 3.4 (s, 3H), 3.5 (q, 2H), 3.7 (s, 3H), 6.03 (br, 1H), 6.72 (s, |
| | | | | | | | 1H), 6.75 (s, 1H) |
| P-65 | —COiPr | Me | Me | OMe | F | cis | 1.1 (d, 6H), 1.6-1.9 (m, 3H), 2.0 (s, 3H), 2.4 (m, 1H), 2.45 (hept, 1H), 2.5 |
| | | | | | | | (m, 1H), 3.4 (m 2H), 3.4 (s, 3H), 3.7 (s, 3H), 6.71 (br, 1H), 6.72 (s, 1H), 6.73 |
| | | | | | | | (s, 1H) |
| P-66 | —COiPr | Me | Me | OMe | F | trans | 1.1 (d, 6H), 1.6-1.9 (m, 3H), 2.0 (s, 3H), 2.1 (m, 1H), 2.4 (m, 1H), 2.5 (hept, |
| | | | | | | | 1H), 3.4 (s, 3H), 3.7 (s, 3H), 6.16 (br, 1H), 6.72 (s, 1H), 6.75 (s, 1H) |
| P-67 | —COiPr | Et | Me | OMe | F | cis | 1.1 (d, 6H), 1.2 (t, 3H), 1.6-1.9 (m, 4H), 2.0 (s, 3H), 2.3-2.4 (m, 1H), 2.45 |
| | | | | | | | (m, 1H), 2.6 (hept, 1H), 3.4 (m, 2H), 3.5 (q, 2H), 3.8 (s, 3H), 6.70 (br, 1H), |
| | | | | | | | 6.73 (s, 1H), 6.86 (s, 1H) |
| P-68 | —CO2Et | allyl | Me | OMe | Me | cis | 1.1 (t, 3H), 1.6-1.9 (m, 3H), 2.0 (s, 3H), 2.1 (m, 1H), 2.2 (s, 3H), 2.4-2.5 (m, |
| | | | | | | | 2H), 3.4 (m, 2H), 3.7 (s, 3H), 4.0 (m, 4H), 5.3 (dt, 2H), 6.0 (m, 1H), 6.62 |
| | | | | | | | (br, 1H), 6.74 (s, 1H), 7.00 (s, 1H) |
| P-69 | —CO2Et | Et | Me | F | OMe | cis | 1.1 (t, 3H), 1.2 (t, 3H), 1.5-2.1 (m, 4H), 2.0 (s, 3H), 3.4 (m, 2H), 3.5 (q, 2H), |
| | | | | | | | 3.7 (s, 3H), 4.1 (q, 2H), 6.70 (br, 1H), 6.72 (s, 1H), 6.88 (s, 1H) |
| P-71 | —CO2Et | allyl | Me | Cl | Me | cis | 1.1 (t, 3H), 1.6-1.9 (m, 3H), 2.0 (s, 3H), 2.1 (m, 1H), 2.2 (s, 3H), 2.4-2.5 (m, |
| | | | | | | | 2H), 3.4 (m, 2H), 4.0 (m, 4H), 5.32 (dt, 2H), 5.95 (dq, 1H), 6.79 (br, 1H), |
| | | | | | | | 6.85 (s, 1H), 7.15 (s, 1H) |
| P-72 | —CO2Et | allyl | Me | Cl | Me | trans | 1.1 (t, 3H), 1.6-1.9 (m, 4H), 2.0 (s, 3H), 2.2 (s, 3H), 2.4-2.5 (m, 2H), 3.4 (m, |
| | | | | | | | 2H), 4.0 (q, 2H), 4.1 (dt, 2H), 5.32 (dq, 2H), 5.95 (m, 1H), 6.35 (br, 1H), |
| | | | | | | | 7.16 (s, 1H), 7.26 (s, 1H) |
| P-75 | —CO2Et | 2- | Me | Cl | Me | cis | 1.1 (t, 3H), 1.6-1.9 (m, 3H), 2.0 (s, 3H), 2.2 (m, 1H), 2.3 (s, 3H), 2.4 (m, |
| | | methoxy- | | | | | 2H), 3.4 (s, 3H), 3.5 (m, 2H), 3.6 (m, 4H), 4.0 (q, 2H), 7.14 (br, 1H), 7.28 (s, |
| | | ethyl | | | | | 1H), 7.29 (s, 1H) |
| P-76 | —CO2Et | 2- | Me | Cl | Me | trans | 1.1 (t, 3H), 1.6-1.9 (m, 4H), 2.0 (s, 3H), 2.2 (m, 1H), 2.3 (s, 3H), 2.4 (m, |
| | | methoxy- | | | | | 1H), 3.4 (s, 3H), 3.5 (m, 2H), 3.6 (m, 4H), 4.0 (q, 2H), 6.35 (br, 1H), 7.16 (s, |
| | | ethyl | | | | | 1H), 7.26 (s, 1H) |
| P-77 | —CO2Et | allyl | Me | OMe | F | trans | 1.1 (t, 3H), 1.6-1.9 (m, 4H), 1.9-2.1 (m, 2H), 2.0 (s, 3H), 2.4-2.5 (m, 2H), 3.4 |
| | | | | | | | (m, 2H), 3.7 (s, 3H), 4.0 (m, 2H), 4.1 (m, 2H), 5.17 (qd, 1H), 5.29 (qd, 1H), |
| | | | | | | | 5.94 (m, 1H), 6.15 (br, 1H), 6.72 (s, 1H), 6.77 (s, 1H) |
| P-78 | —CO2Et | allyl | Me | OMe | F | cis | 1.1 (t, 3H), 1.6-1.8 (m, 3H), 1.9-2.1 (m, 1H), 2.0 (s, 3H), 2.4-2.5 (m, 2H), 3.4 |
| | | | | | | | (m, 2H), 3.7 (s, 3H), 4.0 (m, 4H). 5.22 (qd, 1H), 5.31 (qd, 1H), 5.94 (m, 1H), |
| | | | | | | | 6.68 (br, 1H), 6.72 (s, 1H), 6.77 (s, 1H) |
| P-79 | —CO2Et | 2- | Me | OMe | Me | cis | 1.1 (t, 3H), 1.6-1.1.9 (m, 4H), 2.0 (s, 3H), 2.2 (s, 3H), 2.4-2.5 (m, 2H), 3.4 (s, |
| | | methoxy- | | | | | 3H), 3.5 (m, 2H), 3.6 (m, 4H), 3.7 (s, 3H), 4.0 (q, 2H), 6.73 (br, 1H), 6.88 (s, |
| | | ethyl | | | | | 1H), 7.13 (s, 1H) |
| P-80 | —CO2Et | 2- | Me | OMe | Me | trans | 1.1 (t, 3H), 1.6-1.1.9 (m, 5H), 2.0 (s, 3H), 2.2 (s, 3H), 2.4 (m, 1H), 2.5 (m, |
| | | methoxy- | | | | | 1H), 3.4 (s, 3H), 3.5 (m, 2H), 3.6 (m, 2H), 3.65 (m, 1H), 3.7 (s, 3H), 4.0 (q, |
| | | ethyl | | | | | 2H), 5.96 (br, 1H), 6.75 (s, 1H), 6.89 (s, 1H) |
| P-81 | —CO2Et | propyl | Me | Me | OMe | trans | 0.8 (t, 3H), 1.2 (t, 3H), 1.6-1.9 (m, 4H), 2.0 (s, 3H), 2.2 (s, 3H), 2.3 (m, 1H), |
| | | | | | | | 2.5 (m, 1H), 3.4 (m, 4H), 3.7 (s, 3H), 4.0 (q, 2H), 6.0 (br, 1H), 6.75 (s, 1H), |
| | | | | | | | 6.89 (s, 1H) |
| P-82 | —CO2Et | propyl | Me | F | OMe | cis | 0.9 (t, 3H), 1.2 (t, 3H), 1.6 (m, 3H), 1.7 (m, 2H), 2.0 (s, 3H), 2.1 (m, 1H), 2.5 |
| | | | | | | | (m, 2H), 3.4 (m, 4H), 3.80 (s, 3H), 4.1 (q, 2H), 6.69 (br, 1H), 6.72 (s, 1H), |
| | | | | | | | 6.87 (s, 1H) |
| P-83 | —CO2Et | propyl | Me | Me | OMe | cis | 0.9 (t, 3H), 1.1 (t, 3H), 1.7 (m, 3H), 1.8 (m, 1H), 2.0 (s, 3H), 2.1 (m, 1H), 2.2 |
| | | | | | | | (s, 3H), 2.5 (m, 2H), 3.4 (m, 4H), 3.8 (s, 3H), 4.0 (q, 2H), 6.74 (s, 1H), 6.76 |
| | | | | | | | (s, 1H), 6.89 (s, 1H) |
| P-84 | —CO2Et | propyl | Me | F | OMe | trans | 0.9 (t, 3H), 1.2 (t, 3H), 1.6-1.9 (m, 4H), 2.0 (s, 3H), 2.3 (m, 1H), 2.5 (m, 1H), |
| | | | | | | | 3.4 (m, 4H), 3.80 (s, 3H), 4.1 (q, 2H), 6.0 (br, 1H), 6.72 (s, 1H), 6.78 (s, 1H) |
| P-85 | —CO2Et | allyl | Me | Me | Br | cis | 1.1 (t, 3H), 1.6-1.9 (m, 4H), 2.0 (s, 3H), 2.1 (s. 3H), 2.4-2.5 (m, 2H), 3.5 (m, |
| | | | | | | | 2H), 4.0 (m, 4H), 5.23 (qd, 1H), 5.32 (qd, 1H), 5.93 (m, 1H), 6.82 (s, 1H), |
| | | | | | | | 7.19 (s, 1H), 7.45 (s, 1H) |
| P-86 | —CO2Et | propyl | Me | Cl | Me | trans | 0.9 (t, 3H), 1.1 (t, 3H), 1.6 (m, 4H), 2.0 (s, 3H), 2.3 (s, 3H), 2.4 (m, 2H), 3.4 |
| | | | | | | | (m, 4H), 4.0 (q, 2H), 6.41 (br, 1H), 7.16 (s, 1H), 7.27 (s, 1H) |
| P-87 | —CO2Et | propyl | Me | Cl | Me | cis | 0.9 (t, 3H), 1.2 (t, 3H), 1.7 (m, 3H), 1.9 (m, 2H), 2.0 (s, 3H), 2.2 (s, 3H), 2.5 |
| | | | | | | | (m, 2H), 3.5 (m, 4H), 4.0 (q, 2H), 6.92 (br, 1H), 6.99 (s, 1H), 7.15 (s, 1H) |
| P-88 | —CO2Et | allyl | Me | Me | Br | trans | 1.2 (t, 3H), 1.6-1.9 (m, 4H), 2.0 (s, 3H), 2.1 (s. 3H), 2.4-2.5 (m, 2H), 3.5 (m, |
| | | | | | | | 2H), 4.0 (m, 2H), 4.1 (q, 2H), 5.23 (qd, 1H), 5.32 (qd, 1H), 5.93 (m, 1H), |
| | | | | | | | 6.00 (s, 1H), 7.19 (s, 1H), 7.45 (s, 1H) |
| P-89 | —CO2Et | Et | Me | Br | OEt | trans | 1.1 (t, 3H), 1.2 (t, 3H), 1.3 (t, 3H), 1.6-1.9 (m, 4H), 2.0 (s, 3H), 2.4 (m, 1H), |
| | | | | | | | 2.5 (m, 1H), 3.4 (m, 2H), 3.5 (q, 2H), 3.9 (q, 2H), 4.1 (q, 2H), 6.0 (br, 1H), |
| | | | | | | | 6.82 (s, 1H), 7.24 (s, 1H) |
| P-90 | —CO2Et | Me | Me | Br | OEt | cis | 1.4 (t, 6H), 1.6-1.9 (m, 4H), 2.0 (s, 3H), 2.2 (m, 1H), 2.5 (m, 1H), 3.4 (q, |
| | | | | | | | 2H), 3.5 (s, 3H), 4.0 (q, 2H), 6.82 (br, 1H), 7.22 (s, 1H), 7.26 (s, 1H) |
| P-94 | —CO2Et | Me | Me | Br | OEt | trans | 1.2 (t, 3H), 1.3 (t, 3H), 1.6-1.9 (m, 4H), 2.0 (s, 3H), 2.3 (m, 1H), 2.5 (m, 1H), |
| | | | | | | | 3.4 (m, 2H), 3.4 (s, 3H), 4.0 (q, 2H), 4.1 (q, 2H), 5.92 (br, 1H), 6.82 (s, 1H), |
| | | | | | | | 7.26 (s, 1H) |
| P-95 | —CO2Et | Et | Me | Br | OEt | cis | 1.1 (t, 3H), 1.2 (t, 3H), 1.3 (t, 3H), 1.6-1.9 (m, 3H), 2.0 (s, 3H), 2.1 (m, 1H), |
| | | | | | | | 2.5 (m, 2H), 3.4 (m, 2H), 3.5 (m, 2H), 3.9 (q, 2H), 4.1 (q, 2H), 6.80 (br, 1H), |
| | | | | | | | 6.82 (s, 1H), 6.83 (s, 1H) |
| P-97 | —CO2Et | Me | Me | Cl | OMe | cis | 1.2 (t, 3H), 1.6-1.9 (m, 3H), 2.0 (s, 3H), 2.1 (m, 1H), 2.5 (m, 2H), 3.4 (m, |
| | | | | | | | 1H), 3.5 (s, 3H), 3.7 (s, 3H), 4.0 (q, 2H), 6.73 (br, 1H), 6.84 (s, 1H), 7.97 (s, |
| | | | | | | | 1H) |
| P-98 | —CO2Et | Me | Me | Cl | OMe | trans | 1.2 (t, 3H), 1.6-1.9 (m, 4H), 2.0 (s, 3H), 2.4-2.5 (m, 2H), 3.4 (m, 1H), 3.5 (s, |
| | | | | | | | 3H), 3.7 (s, 3H), 4.0 (q, 2H), 6.16 (br, 1H), 6.84 (s, 1H), 7.07 (s, 1H) |
| P-99 | —CO2Et | prop-2- | Me | Cl | Me | trans | 1.1 (t, 3H), 1.6-1.9 (m, 4H), 2.0 (s, 3H), 2.2 (s, 3H), 2.3 (m, 1H), 2.4-2.5 (m, |
| | | yn-1-yl | | | | | 2H), 3.5 (m, 2H), 4.0 (q, 2H), 4.1 (s, 3H), 6.19 (br, 1H), 7.16 (s, 1H), 7.26 (s, |
| | | | | | | | 1H) |
| P- | —CO2Et | prop-2- | Me | Cl | Me | cis | 1.1 (t, 3H), 1.7-1.9 (m, 3H), 2.0 (s, 3H), 2.1 (m, 1H), 2.2 (s, 3H), 2.4-2.5 (m, |
| 100 | | yn-1-yl | | | | | 2H), 3.5 (m, 2H), 4.0 (q, 2H), 4.2 (m, 2H), 6.63 (s, 1H), 6.66 (s, 1H), 7.15 |
| | | | | | | | (s, 1H) |
| P- | —CO2Et | prop-2- | Me | OMe | Me | cis | 1.1 (t, 3H), 1.6-1.9 (m, 3H), 2.0 (s, 3H), 2.1 (m, 1H), 2.2 (s, 3H), 2.4-2.5 (m, |
| 101 | | yn-1-yl | | | | | 2H), 3.5 (m, 2H), 3.7 (s, 3H), 4.0 (m, 2H), 4.2 (m, 2H), 6.54 (br, 1H), 6.75 (s, |
| | | | | | | | 1H), 6.89 (s, 1H) |
| P- | —CO2Et | prop-2- | Me | OMe | F | trans | 1.1 (t, 3H), 1.6-1.9 (m, 4H), 2.0 (s, 3H), 2.3 (m, 1H), 2.4-2.5 (m, 2H), 3.5 (m, |
| 102 | | yn-1-yl | | | | | 2H), 3.7 (s, 3H), 4.0 (q, 2H), 4.1 (s, 1H), 5.91 (br, 1H), 6.76 (s, 1H), 6.78 (s, |
| | | | | | | | 1H) |
| P- | —CO2Et | prop-2- | Me | OMe | Me | trans | 1.1 (t, 3H), 1.6-1.9 (m, 4H), 2.0 (s, 3H), 2.2 (s, 3H), 2.3 (m, 1H), 2.4-2.5 (m, |
| 103 | | yn-1-yl | | | | | 2H), 3.5 (m, 2H), 3.7 (s, 3H), 4.0 (q, 2H), 4.1 (s, 1H), 6.0 (br, 1H), 6.75 (s, |
| | | | | | | | 1H), 6.89 (s, 1H) |
| P- | —CO2Et | prop-2- | Me | OMe | F | cis | 1.1 (t, 3H), 1.6-1.9 (m, 3H), 2.0 (s, 3H), 2.2 (s, 3H), 2.3 (m, 1H), 2.4-2.5 (m, |
| 104 | | yn-1-yl | | | | | 2H), 3.5 (m, 2H), 3.7 (s, 3H), 4.0 (q, 2H), 4.1 (s, 1H), 6.7 (br, 1H), 6.72 (s, |
| | | | | | | | 1H), 6.74 (s, 1H) |
| P- | —CO2Et | allyl | Me | Me | F | cis | 1.1 (t, 3H), 1.6-1.9 (m, 3H), 2.0 (s, 3H), 2.1 (m, 1H), 2.2 (s, 3H), 2.4-2.5 (m, |
| 105 | | | | | | | 2H), 3.5 (m, 2H), 4.0 (m, 4H), 5.23 (qd, 1H), 5.94 (qd, 1H), 6.00 (m, 1H), |
| | | | | | | | 6.77 (br, 1H), 6.91 (s, 1H), 7.05 (s, 1H) |

Preparation examples for starting materials:

### Example A.6 Methyl 1-[[2-(2-bromo-6-methoxy-4-prop-1-ynylphenyl)acetyl]amino]-3-(methoxymethyl)cyclopentanecarboxylate

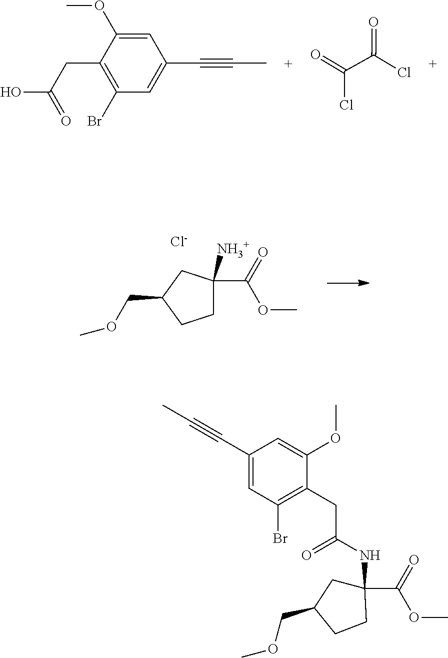

0.90 g (3.17 mmol) of [2-bromo-6-methoxy-4-(prop-1-yn-1-yl)phenyl]acetic acid was dissolved in 3.33 ml of dichloromethane and 2 drops of dimethylformamide were added. At room temperature, 0.55 ml (6.35 mmol) of oxalyl chloride was slowly added dropwise and the mixture was then heated at reflux until the evolution of gas had ceased and concentrated. In a separate reaction, the acid chloride dissolved in dichloromethane was added dropwise to an initial charge of 0.77 g (3.17 mmol) of [1-methoxycarbonyl-3-(methoxymethyl)cyclopentyl]ammonium chloride and 1.72 ml (12.7 mmol) of triethylamine in 3.3 ml of dichloromethane. Stirring at room temperature was continued for 1 h.

The mixture was washed with water, the phases were separated and the organic phase was dried over sodium sulfate. After concentrating, the residue was purified by chromatography.

This gave 1 g of a light-colored oil (76% yield).

Analogously to this example and also according to the general details relating to the preparation, the following compounds are obtained:

| Example | | |
|---|---|---|
| No. | Structure | ¹H NMR (400 MHz, CDCl₃) |
| A1 | 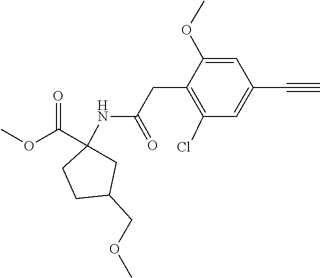 [SMILES: C#Cc1cc(Cl)c(CC(=O)NC2(C(=O)OC)CCC(COC)C2)c(OC)c1] | 1.55-1.61 (m, 1H), 1.68-1.74 (m, 1H), 1.81-1.89 (m, 1H), 2.04-2.22 (m, 3H), 2.35 (m, 1H), 3.10 (s, 1H), 3.22 (m, 2H), 3.25 (s, 3H), 3.69 (s, 3H), 3.73 (s, 3H), 3.85 (s, 3H), 6.49 (br, NH), 6.91 (s, 1H), 7.19 (s, 1H) |
| A2 | 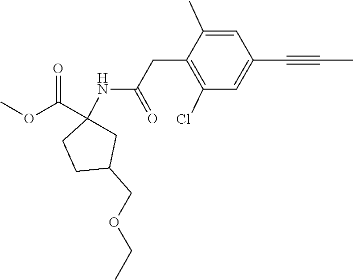 [SMILES: CC#Cc1cc(C)c(CC(=O)NC2(C(=O)OC)CCC(COCC)C2)c(Cl)c1] | 1.17 (t, 3H), 1.48-1.51 (m, 1H), 1.79-1.93 (m, 3H), 2.05 (s, 3H), 2.18-2.22 (m, 2H), 2.34 (s, 3H), 3.34 (q, 2H), 3.46 (q, 2H), 3.67 (s, 3H), 3.73 (s, 2H), 5.67 (br, NH), 7.16 (s, 1H), 7.31 (s, 1H) |
| A3 | 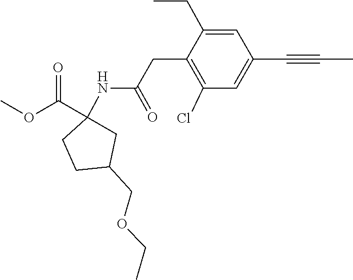 [SMILES: CC#Cc1cc(Cl)c(CC(=O)NC2(C(=O)OC)CCC(COCC)C2)c(CC)c1] | 1.15 (t, 3H), 1.23 (t, 3H), 1.50 (m, 1H), 1.85-1.93 (m 3H), 2.05 (s, 3H), 2.18-2.33 (m, 2H), 2.67 (q, 2H), 3.33 (m, 2H), 3.45 (q, 2H), 3.67 (s, 3H), 3.74 (s, 2H), 5.85 (br, NH), 7.19 (s, 1H), 7.32 (s, 1H) |
| A4 | 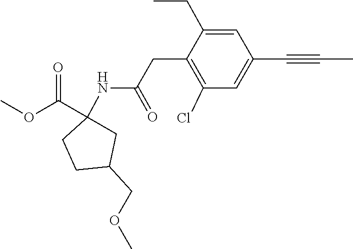 [SMILES: CC#Cc1cc(Cl)c(CC(=O)NC2(C(=O)OC)CCC(COC)C2)c(CC)c1] | 1.21 (t, 3H), 1.80 (m, 1H), 1.88-2.04 (m, 2H), 2.05 (s, 3H), 2.16-2.20 (m, 2H), 2.69 (m, 2H), 3.26-3.34 (m, 1H), 3.31 (s, 3H), 3.67 (s, 3H), 3.74 (s, 2H), 5.66 (br, NH), 7.19 (s, 1H), 7.32 (s, 1H) |
| A5 | 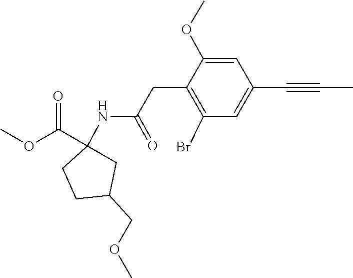 [SMILES: CC#Cc1cc(Br)c(CC(=O)NC2(C(=O)OC)CCC(COC)C2)c(OC)c1] | 1.52-1.88 (m, 3H), 2.05 (s, 3H), 2.17 (m, 2H), 2.35- 2.41(m, 1H), 3.32 (s, 3H), 3.33 (m, 1H), 3.69 (s, 3H), 3.74 (s, 2H), 3.82 (s, 3H), 6.41 (br, NH), 6.86 (s, 1H), 7.26 (s, 1H) |
| A6 | 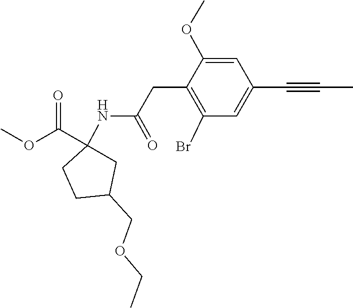 [SMILES: CC#Cc1cc(Br)c(CC(=O)NC2(C(=O)OC)CCC(COCC)C2)c(OC)c1] | 1.21 (t, 3H), 1.59-1.73 (m, 2H), 1.75 (m, 1H), 2.04 (s, 3H), 2.18-2.45 (m, 3H), 3.33 (m, 2H), 3.48 (q, 2H), 3.68 (s, 3H), 3.82 (s, 2H), 6.56 (br, NH), 6.85 (s, 1H), 7.25 (s, 1H) |
| A7 | 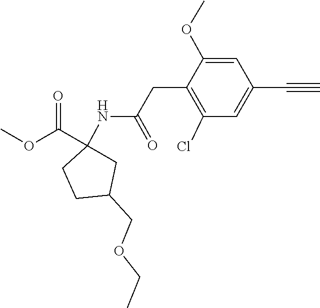 [SMILES: C#Cc1cc(Cl)c(CC(=O)NC2(C(=O)OC)CCC(COCC)C2)c(OC)c1] | 1.17 (t, 3H), 1.49-1.61 (m, 1H), 1.82-1.97 (m, 3H), 2.04-2.12 (m, 1H), 2.13-2.20 (m, 1H), 2.20-2.41 (m, 1H), 3.33 (q, 2H), 3.46 (q, 2H), 3.68 (s, 3H), 3.73 (s, 2H), 3.86 (s, 3H), 5.86 (br, NH), 6.92 (s, 1H), 7.19 (s, 1H) |

### 2-Fluoro-6-methyl-4-nitroaniline

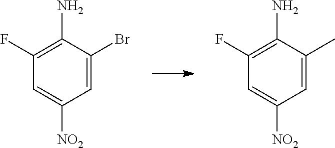

To a solution of dimethyl ether (100 ml) and water (60 ml) were added 150 mg (0.64 mmol) of 2-bromo-6-fluoro-4-nitroaniline (CAS number: 455-58-3), 200 mg (1.91 mmol) of sodium carbonate and trimethylboroxine (240 mg, 0.95 mmol) and Pd(dppd)Cl₂ (18.6 mg, 0.02 mmol) under argon, and the mixture was heated at 90° C. for 16 h. The mixture was concentrated to dryness and the residue was dissolved in water and extracted with dichloromethane. The extract was dried with sodium sulfate, concentrated and chromatographed. This gave 188 mg (55%).

¹H NMR (400 MHz, δ in ppm, CDCl₃)

δ=2.26 (s, 3H), 4.36 (br, NH2), 7.81 (s, 1H), 7.86 (s, 1H)

### 1-Fluoro-3-methyl-5-nitro-2-(2,2,2-trichloroethyl)benzene

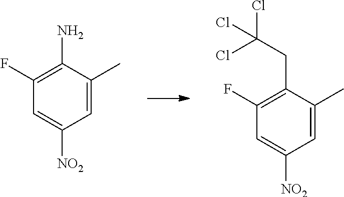 [SMILES: Cc1cc([N+](=O)[O-])cc(F)c1N ; Cc1cc([N+](=O)[O-])cc(F)c1CC(Cl)(Cl)Cl]

132 mg (1.28 mmol) of tert-butyl nitrite and 144 mg (1.06 mmol) of copper(II) chloride were suspended in 7.8 ml of acetonitrile and cooled to 0° C. Then 983 mg (10.1 mmol) of vinylidene chloride was slowly added dropwise and the mixture was allowed to warm to room temperature. Subsequently, 123 mg (0.72 mmol) of 2-fluoro-6-methyl-4-nitroaniline, dissolved in 10 ml of acetonitrile and 25 ml of acetone, was slowly added dropwise. Stirring at room temperature was continued until the evolution of gas had ceased.

While cooling with ice, the mixture was slowly added to 2 ml of 10% aqueous hydrochloric acid and extracted with ethyl acetate, and the extract was dried with magnesium sulfate and concentrated. This gave 3.636 g of a crude product which still contained copper salts and was used directly for the next reaction.

¹H NMR (400 MHz, δ in ppm, CDCl₃)

δ=2.66 (s, 3H), 1.96 (s, 2H), 7.83 (s, 1H), 7.96 (s, 1H)

### Methyl 2-(2-fluoro-6-methyl-4-nitrophenyl)acetate

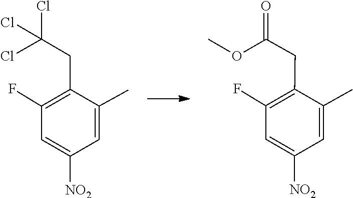

160 mg (0.55 mmol) of 1-fluoro-3-methyl-5-nitro-2-(2,2,2-trichloroethyl)benzene was dissolved in 10 ml of methanol, and 121 mg (2.23 mmol) of 30% methanolic sodium methoxide solution was added slowly, resulting in evolution of heat. The mixture was then heated at reflux for 12 h. 1.1 ml of concentrated sulfuric acid was added cautiously, resulting in evolution of heat. The mixture was heated under reflux for 1 h. The mixture was concentrated to dryness and the residue was dissolved in water and extracted with dichloromethane. The extract was dried with sodium sulfate, concentrated and chromatographed. This gave 110 mg of a yellow oil (75% yield).

¹H NMR (400 MHz, δ in ppm, CDCl₃)

δ=2.42 (s, 3H), 3.72 (s, 3H), 3.78 (s, 2H), 7.78 (s, 1H), 7.91 (s, 1H)

### Methyl 2-(4-amino-2-fluoro-6-methylphenyl)acetate

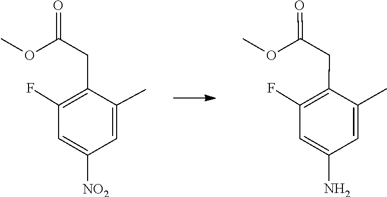

1.40 g (6.16 mmol) of methyl (2-fluoro-6-methyl-4-nitrophenyl)acetate was dissolved in 11 ml of tetrahydrofuran, and a solution of 2.040 g (38.1 mmol) of ammonium chloride in 5.3 ml of water and 1.37 g (2.46 mmol) of iron powder were added. The mixture was stirred at room temperature for 30 min. The mixture was filtered, the filtrate was diluted with water and extracted with ethyl acetate, adjusting the pH to greater than 7. The extract was dried with sodium sulfate and concentrated. 1.17 g a brown substance was obtained (90% yield).

### Methyl 2-(2-fluoro-4-Iodo-6-methylphenyl)acetate

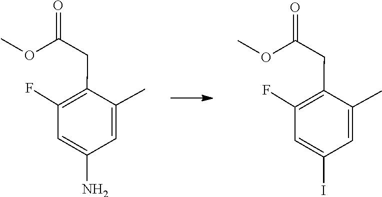

A suspension of 1.40 g (6.16 mmol) of methyl (4-amino-2-fluoro-6-methylphenyl)acetate and 3 ml (3.76 mol) of HCl in 30 ml of water was cooled to −5-0° C., and a solution of 0.4 g (6.03 mmol) of sodium nitrite and 0.8 g (11.08 mmol) of potassium iodide in 1.8 ml of water was added gradually. After 10 min, the mixture was warmed to room temperature and stirred at 20° C. for a further 30 min. 15 ml of water was added, the pH was adjusted to pH 8 with saturated sodium hydrogencarbonate solution, and then saturated sodium thiosulfate solution was added. After extraction with ethyl acetate, drying (sodium sulfate) and distillative removal of the solvent, the residue was purified by chromatography on silica gel (ethyl acetate/hexane). This gave 1.35 g (75%) of the iodine compound as a white solid.

¹H NMR (400 MHz, δ in ppm, CDCl₃)

δ=2.26 (s, 3H), 3.64 (s, 2H), 3.70 (s, 3H), 7.29 (s, 1H), 7.35 (s, 1H)

### Example B.8

### Methyl 2-(2-fluoro-6-methyl-4-prop-1-ynylphenyl)acetate

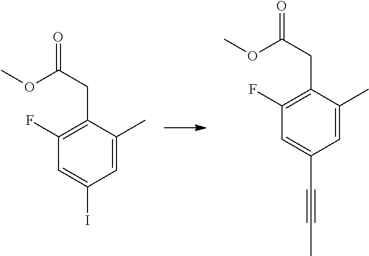

To a solution of 0.102 g (0.75 mmol) of zinc chloride and 0.032 g (0.75 mmol) of lithium chloride in 7 ml of dry tetrahydrofuran under nitrogen at 0° C. was added dropwise, while stirring, 1.5 ml (0.75 mmol) of a 0.5 M solution of 1-propylmagnesium bromide in tetrahydrofuran. The solution was warmed to room temperature while stirring within 1.5 h (solution 1). 2.8 mg (0.01 mmol) of palladium(II) acetate and 10.6 mg (0.02 mmol) of 1,4-bis(diphenylphosphino)butane in 3 ml of dry tetrahydrofuran were stirred under a nitrogen atmosphere at room temperature for 30 min (solution 2).

0.14 g (0.5 mmol) of methyl (2-fluoro-6-methyl-4-iodophenyl)acetate was dissolved in 2 ml of dry tetrahydrofuran under a nitrogen atmosphere, and the mixture was stirred at room temperature for 30 min (solution 3).

Solution 2 and then solution 3 were each added dropwise at room temperature to solution 1 while stirring and, after the addition had ended, the mixture was stirred at 60° C. for 3.5 h.

After cooling to room temperature, water and saturated ammonium chloride solution were added, the mixture was extracted with ethyl acetate, the organic phase was dried (sodium sulfate), and the solvent was distilled off. Chromatography on silica gel (ethyl acetate/hexane) gave 75 mg of the desired compound (65% yield).

¹H NMR (400 MHz, δ in ppm, CDCl₃):

δ=2.04 (s, 3H), 2.23 (s, 3H), 3.62 (s, 3H), 3.71 (s, 2H), 7.04 (s, 1H), 7.10 (s, 1H)

The following precursors were prepared analogously:

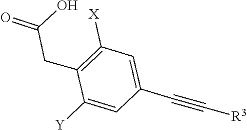

TABLE 3
Compounds of the general formula (X)

| Example No. | R³ | X | Y | ¹H NMR (400 MHz, δ in ppm) |
|---|---|---|---|---|
| B.1 | Me | Cl | Me | (d₆-DMSO): δ = 2.02 and 2.26 (each s, each 3H), 3.72 (s, 2H), 7.11 and 7.19 |
| | | | | (each s, each 1H) |
| B.2 | Me | Cl | Et | (CDCl₃): δ = 1.21 (t, 3H), 2.10 (s, 3H), 2.67 (q, 2H), 3.92 (s, 2H), 7.06 and |
| | | | | 7.34 (each s, each 1H) |
| B.3 | Me | Br | Me | (d₆-DMSO): δ = 2.05 and 2.28 (each s, each 3H), 3.78 (s, 2H), 7.26 and 7.45 |
| | | | | (each s, each 1H) |
| B.4 | Me | Br | OMe | (CDCl₃): δ = 2.05 (s, 3H), 3.80 (s, 3H), 3.90 (s, 2H), 6.80 (s, 1H), 7.25 (s, |
| | | | | 1H) |
| B.5 | Me | Br | OEt | (d₆-DMSO): δ = 1.30 (t, 3H), 2.05 (s, 3H), 3.65 (s, 2H), 4.05 (q, 2H), 7.00 (s, |
| | | | | 1H), 7.20 (s, 1H) |
| B.6 | H | Br | OMe | (d₆-DMSO): δ = 3.70 (s, 2H), 3.80 (s, 3H), 4.30 (s, 1H), 7.10 (s, 1H), 7.30 (s, |
| | | | | 1H) |
| B.7 | Me | Cl | OMe | (CDCl₃): δ = 2.04 (s, 3H), 3.80 (s, 3H), 3.85 (s, 2H), 6.80 (s, 1H), 7.06 (s, |
| | | | | 1H) |
| B.8 | Me | F | Me | (CDCl₃): δ = 2.04 (s, 3H), 2.27 (s, 3H) 3.71 (s, 2H), 6.93 (s, 1H), 7.02 (s, |
| | | | | 1H) |

### B. Formulation Examples

a) A dusting product is obtained by mixing 10 parts by weight of a compound of the formula (I) and/or salts thereof and 90 parts by weight of talc as inert substance and comminuting the mixture in an impact mill.
b) A readily water-dispersible, wettable powder is obtained by mixing 25 parts by weight of a compound of the formula (I) and/or salts thereof, 64 parts by weight of kaolin-containing quartz as inert substance, 10 parts by weight of potassium lignosulfonate and 1 part by weight of sodium oleoylmethyltaurate as wetting agent and dispersant and grinding in a pinned-disc mill.
c) A readily water-dispersible dispersion concentrate is obtained by mixing 20 parts by weight of a compound of the formula (I) and/or salts thereof with 6 parts by weight of alkylphenol polyglycol ether (®Triton X 207), 3 parts by weight of isotridecanol polyglycol ether (8 EO) and 71 parts by weight of paraffinic mineral oil (boiling range e.g. about 255 to more than 277° C.) and grinding to a fineness of below 5 microns in an attrition ball mill.
d) An emulsifiable concentrate is obtained from 15 parts by weight of a compound of the formula (I) and/or salts thereof, 75 parts by weight of cyclohexanone as solvent and 10 parts by weight of oxyethylated nonylphenol as emulsifier.
e) Water-dispersible granules are obtained by mixing
75 parts by weight of a compound of the formula (I) and/or salts thereof,
10 parts by weight of calcium lignosulfonate,
5 parts by weight of sodium laurylsulfate,
3 parts by weight of polyvinyl alcohol and
7 parts by weight of kaolin,
grinding the mixture in a pinned-disk mill, and granulating the powder in a fluidized bed by spray application of water as a granulating liquid.
f) Water-dispersible granules are also obtained by homogenizing and precomminuting, in a colloid mill,
25 parts by weight of a compound of the formula (I) and/or salts thereof,
5 parts by weight of sodium 2,2′-dinaphthylmethane-6,6′-disulfonate,
2 parts by weight of sodium oleoylmethyltaurate,
1 part by weight of polyvinyl alcohol,
17 parts by weight of calcium carbonate and
50 parts by weight of water,
then grinding the mixture in a bead mill and atomizing and drying the resulting suspension in a spray tower by means of a one-phase nozzle.

### C. Biological Examples

1. Pre-Emergence Herbicidal Effect and Crop Plant Compatibility

Seeds of monocotyledonous and dicotyledonous weed plants and crop plants are laid out in sandy loam soil in wood-fiber pots and covered with soil. The compounds of the invention, formulated in the form of wettable powders (WP) or as emulsion concentrates (EC), are then applied to the surface of the covering soil as aqueous suspension or emulsion at a water application rate equating to 600 to 800 L/ha with addition of 0.2% wetting agent.

After the treatment, the pots are placed in a greenhouse and kept under good growth conditions for the trial plants. The damage to the test plants is scored visually after a test period of 3 weeks by comparison with untreated controls (herbicidal activity in percent (%): 100% activity=the plants have died, 0% activity=like control plants).

Undesired Plants/Weeds:

| | | | |
|---|---|---|---|
| ALOMY: | Alopecurus myosuroides | SETVI: | Setaria viridis |
| AMARE: | Amaranthus retroflexus | AVEFA: | Avena fatua |
| CYPES: | Cyperus esculentus | ECHCG: | Echinochloa crus-galli |
| LOLRI: | Lolium rigidum | STEME: | Stellaria media |
| VERPE: | Veronica persica | VIOTR: | Viola tricolor |
| POLCO: | Polygonum convolvulus | ABUTH: | Abutylon threophrasti |
| HORMU: | Hordeum murinum | DIGSA: | Digitaria sanguinalis |

1. Pre-Emergence Effectiveness

As the results from Tables 3 and 4 show, compounds of the invention have a good herbicidal pre-emergence effectiveness against a broad spectrum of weed grasses and weeds.

TABLE 4
Pre-emergence action at
320 g/ha against ALOMY in %

| Example | Dosage | |
|---|---|---|
| number | [g/ha] | ALOMY |
| I-02 | 320 | 100 |
| I-05 | 320 | 100 |
| I-10 | 320 | 100 |
| I-11 | 320 | 100 |
| I-17 | 320 | 100 |
| I-18 | 320 | 100 |
| I-19 | 320 | 100 |
| I-20 | 320 | 100 |
| I-21 | 320 | 100 |
| I-30 | 320 | 100 |
| I-31 | 320 | 100 |
| I-32 | 320 | 100 |
| I-33 | 320 | 100 |
| I-35 | 320 | 100 |
| I-36 | 320 | 100 |
| I-37 | 320 | 80 |
| I-40 | 320 | 100 |
| I-43 | 320 | 100 |
| I-44 | 320 | 100 |
| I-45 | 320 | 80 |
| I-46 | 320 | 100 |
| I-47 | 320 | 100 |
| I-48 | 320 | 100 |
| I-49 | 320 | 100 |
| I-50 | 320 | 100 |
| I-51 | 320 | 100 |
| I-52 | 320 | 100 |
| I-54 | 320 | 100 |
| I-55 | 320 | 100 |
| I-56 | 320 | 80 |
| I-57 | 320 | 100 |
| I-58 | 320 | 100 |
| I-59 | 320 | 100 |
| I-61 | 320 | 100 |
| I-62 | 320 | 100 |
| I-63 | 320 | 100 |
| I-64 | 320 | 100 |
| I-66 | 320 | 100 |
| I-67 | 320 | 100 |
| I-68 | 320 | 100 |
| I-69 | 320 | 100 |
| I-70 | 320 | 100 |
| I-71 | 320 | 100 |
| I-72 | 320 | 100 |
| I-73 | 320 | 100 |
| I-74 | 320 | 100 |
| I-75 | 320 | 90 |
| I-76 | 320 | 100 |
| I-77 | 320 | 100 |
| I-78 | 320 | 100 |
| I-79 | 320 | 100 |
| P-01 | 320 | 100 |
| P-04 | 320 | 100 |
| P-05 | 320 | 100 |
| P-08 | 320 | 100 |
| P-100 | 320 | 100 |
| P-101 | 320 | 100 |
| P-102 | 320 | 100 |
| P-103 | 320 | 100 |
| P-104 | 320 | 100 |
| P-13 | 320 | 100 |
| P-23 | 320 | 100 |
| P-28 | 320 | 100 |
| P-29 | 320 | 100 |
| P-30 | 320 | 100 |
| P-31 | 320 | 100 |
| P-32 | 320 | 100 |
| P-33 | 320 | 100 |
| P-34 | 320 | 100 |
| P-35 | 320 | 100 |
| P-36 | 320 | 100 |
| P-37 | 320 | 100 |
| P-46 | 320 | 100 |
| P-47 | 320 | 100 |
| P-48 | 320 | 100 |
| P-49 | 320 | 100 |
| P-50 | 320 | 100 |
| P-51 | 320 | 100 |
| P-52 | 320 | 100 |
| P-53 | 320 | 100 |
| P-54 | 320 | 100 |
| P-55 | 320 | 100 |
| P-56 | 320 | 100 |
| P-57 | 320 | 100 |
| P-58 | 320 | 100 |
| P-59 | 320 | 100 |
| P-60 | 320 | 100 |
| P-61 | 320 | 100 |
| P-62 | 320 | 100 |
| P-63 | 320 | 100 |
| P-64 | 320 | 100 |
| P-65 | 320 | 100 |
| P-66 | 320 | 100 |
| P-67 | 320 | 100 |
| P-68 | 320 | 100 |
| P-69 | 320 | 100 |
| P-70 | 320 | 100 |
| P-71 | 320 | 100 |
| P-72 | 320 | 90 |
| P-75 | 320 | 100 |
| P-76 | 320 | 100 |
| P-77 | 320 | 100 |
| P-78 | 320 | 100 |
| P-79 | 320 | 100 |
| P-80 | 320 | 100 |
| P-81 | 320 | 100 |
| P-82 | 320 | 100 |
| P-83 | 320 | 100 |
| P-84 | 320 | 100 |
| P-85 | 320 | 100 |
| P-86 | 320 | 100 |
| P-87 | 320 | 100 |
| P-88 | 320 | 100 |
| P-89 | 320 | 100 |
| P-90 | 320 | 100 |
| P-94 | 320 | 100 |
| P-95 | 320 | 100 |
| P-97 | 320 | 100 |
| P-98 | 320 | 100 |
| P-99 | 320 | 100 |

TABLE 5
Pre-emergence action at
320 g/ha against AVEFA in %

| Example | Dosage | |
|---|---|---|
| number | [g/ha] | AVEFA |
| I-02 | 320 | 100 |
| I-05 | 320 | 100 |
| I-10 | 320 | 100 |
| I-11 | 320 | 100 |
| I-17 | 320 | 100 |
| I-18 | 320 | 100 |
| I-19 | 320 | 100 |
| I-20 | 320 | 100 |
| I-21 | 320 | 100 |
| I-30 | 320 | 90 |
| I-31 | 320 | 80 |
| I-32 | 320 | 90 |
| I-33 | 320 | 80 |
| I-36 | 320 | 80 |
| I-40 | 320 | 100 |
| I-41 | 320 | 80 |
| I-42 | 320 | 90 |
| I-43 | 320 | 100 |
| I-44 | 320 | 90 |
| I-46 | 320 | 90 |
| I-47 | 320 | 90 |
| I-48 | 320 | 90 |
| I-49 | 320 | 90 |
| I-50 | 320 | 100 |
| I-51 | 320 | 100 |
| I-52 | 320 | 100 |
| I-54 | 320 | 80 |
| I-58 | 320 | 80 |
| I-61 | 320 | 90 |
| I-62 | 320 | 100 |
| I-63 | 320 | 100 |
| I-64 | 320 | 80 |
| I-66 | 320 | 100 |
| I-67 | 320 | 80 |
| I-68 | 320 | 100 |
| I-69 | 320 | 90 |
| I-70 | 320 | 100 |
| I-71 | 320 | 80 |
| I-72 | 320 | 100 |
| I-73 | 320 | 100 |
| I-74 | 320 | 90 |
| I-75 | 320 | 100 |
| I-77 | 320 | 100 |
| I-78 | 320 | 90 |
| I-79 | 320 | 100 |
| P-01 | 320 | 100 |
| P-04 | 320 | 100 |
| P-05 | 320 | 100 |
| P-08 | 320 | 100 |
| P-100 | 320 | 90 |
| P-101 | 320 | 100 |
| P-102 | 320 | 80 |
| P-103 | 320 | 100 |
| P-13 | 320 | 100 |
| P-23 | 320 | 100 |
| P-28 | 320 | 100 |
| P-29 | 320 | 100 |
| P-30 | 320 | 100 |
| P-31 | 320 | 100 |
| P-32 | 320 | 90 |
| P-33 | 320 | 100 |
| P-34 | 320 | 100 |
| P-35 | 320 | 100 |
| P-36 | 320 | 80 |
| P-37 | 320 | 80 |
| P-46 | 320 | 100 |
| P-47 | 320 | 100 |
| P-48 | 320 | 100 |
| P-49 | 320 | 100 |
| P-50 | 320 | 100 |
| P-51 | 320 | 80 |
| P-52 | 320 | 90 |
| P-53 | 320 | 90 |
| P-54 | 320 | 90 |
| P-55 | 320 | 80 |
| P-56 | 320 | 80 |
| P-57 | 320 | 80 |
| P-58 | 320 | 90 |
| P-59 | 320 | 100 |
| P-60 | 320 | 100 |
| P-61 | 320 | 90 |
| P-62 | 320 | 90 |
| P-63 | 320 | 90 |
| P-64 | 320 | 80 |
| P-65 | 320 | 100 |
| P-66 | 320 | 100 |
| P-67 | 320 | 80 |
| P-69 | 320 | 80 |
| P-70 | 320 | 90 |
| P-71 | 320 | 90 |
| P-75 | 320 | 100 |
| P-76 | 320 | 100 |
| P-77 | 320 | 90 |
| P-79 | 320 | 100 |
| P-80 | 320 | 100 |
| P-81 | 320 | 100 |
| P-82 | 320 | 90 |
| P-83 | 320 | 80 |
| P-84 | 320 | 80 |
| P-85 | 320 | 80 |
| P-86 | 320 | 80 |
| P-87 | 320 | 100 |
| P-88 | 320 | 90 |
| P-89 | 320 | 100 |
| P-90 | 320 | 100 |
| P-94 | 320 | 100 |
| P-95 | 320 | 100 |
| P-97 | 320 | 100 |
| P-98 | 320 | 100 |
| P-99 | 320 | 100 |

TABLE 6
Pre-emergence action at
320 g/ha against DIGSA in %

| | | |
|---|---|---|
| I-02 | 320 | 100 |
| I-05 | 320 | 100 |
| I-10 | 320 | 100 |
| I-11 | 320 | 100 |
| I-17 | 320 | 100 |
| I-18 | 320 | 100 |
| I-19 | 320 | 100 |
| I-20 | 320 | 100 |
| I-21 | 320 | 100 |
| I-30 | 320 | 100 |
| I-31 | 320 | 100 |
| I-32 | 320 | 100 |
| I-33 | 320 | 100 |
| I-34 | 320 | 100 |
| I-35 | 320 | 100 |
| I-36 | 320 | 100 |
| I-37 | 320 | 90 |
| I-40 | 320 | 100 |
| I-41 | 320 | 100 |
| I-42 | 320 | 100 |
| I-43 | 320 | 100 |
| I-44 | 320 | 100 |
| I-45 | 320 | 100 |
| I-46 | 320 | 100 |
| I-47 | 320 | 100 |
| I-48 | 320 | 100 |
| I-49 | 320 | 100 |
| I-50 | 320 | 100 |
| I-51 | 320 | 100 |
| I-52 | 320 | 100 |
| I-54 | 320 | 100 |
| I-55 | 320 | 100 |
| I-56 | 320 | 100 |
| I-57 | 320 | 100 |
| I-58 | 320 | 100 |
| I-59 | 320 | 100 |
| I-61 | 320 | 100 |
| I-62 | 320 | 100 |
| I-63 | 320 | 100 |
| I-64 | 320 | 100 |
| I-66 | 320 | 100 |
| I-67 | 320 | 100 |
| I-68 | 320 | 100 |
| I-69 | 320 | 100 |
| I-70 | 320 | 100 |
| I-71 | 320 | 100 |
| I-72 | 320 | 100 |
| I-73 | 320 | 100 |
| I-74 | 320 | 100 |
| I-75 | 320 | 100 |
| I-76 | 320 | 100 |
| I-77 | 320 | 100 |
| I-78 | 320 | 100 |
| I-79 | 320 | 100 |
| P-01 | 320 | 100 |
| P-04 | 320 | 100 |
| P-05 | 320 | 100 |
| P-08 | 320 | 100 |
| P-100 | 320 | 100 |
| P-101 | 320 | 100 |
| P-102 | 320 | 100 |
| P-103 | 320 | 100 |
| P-104 | 320 | 100 |
| P-13 | 320 | 100 |
| P-23 | 320 | 100 |
| P-28 | 320 | 100 |
| P-29 | 320 | 100 |
| P-30 | 320 | 100 |
| P-31 | 320 | 100 |
| P-32 | 320 | 100 |
| P-33 | 320 | 100 |
| P-34 | 320 | 100 |
| P-35 | 320 | 100 |
| P-36 | 320 | 100 |
| P-37 | 320 | 100 |
| P-46 | 320 | 100 |
| P-47 | 320 | 100 |
| P-48 | 320 | 100 |
| P-49 | 320 | 100 |
| P-50 | 320 | 100 |
| P-51 | 320 | 100 |
| P-52 | 320 | 100 |
| P-53 | 320 | 100 |
| P-54 | 320 | 100 |
| P-55 | 320 | 100 |
| P-56 | 320 | 100 |
| P-57 | 320 | 100 |
| P-58 | 320 | 100 |
| P-59 | 320 | 100 |
| P-60 | 320 | 100 |
| P-61 | 320 | 100 |
| P-62 | 320 | 100 |
| P-63 | 320 | 100 |
| P-64 | 320 | 100 |
| P-65 | 320 | 100 |
| P-66 | 320 | 100 |
| P-67 | 320 | 100 |
| P-68 | 320 | 100 |
| P-69 | 320 | 100 |
| P-70 | 320 | 100 |
| P-71 | 320 | 100 |
| P-72 | 320 | 100 |
| P-75 | 320 | 100 |
| P-76 | 320 | 100 |
| P-77 | 320 | 100 |
| P-78 | 320 | 100 |
| P-79 | 320 | 100 |
| P-80 | 320 | 100 |
| P-81 | 320 | 100 |
| P-82 | 320 | 100 |
| P-83 | 320 | 100 |
| P-84 | 320 | 100 |
| P-85 | 320 | 100 |
| P-86 | 320 | 100 |
| P-87 | 320 | 100 |
| P-88 | 320 | 100 |
| P-89 | 320 | 100 |
| P-90 | 320 | 100 |
| P-94 | 320 | 100 |
| P-95 | 320 | 100 |
| P-97 | 320 | 100 |
| P-98 | 320 | 100 |
| P-99 | 320 | 100 |

TABLE 7
Pre-emergence action at 320 g/ha against ECHCG in %

| Example | Dosage | |
|---|---|---|
| number | [g/ha] | ECHCG |
| I-02 | 320 | 100 |
| I-05 | 320 | 100 |
| I-10 | 320 | 100 |
| I-11 | 320 | 100 |
| I-17 | 320 | 100 |
| I-18 | 320 | 100 |
| I-19 | 320 | 100 |
| I-20 | 320 | 100 |
| I-21 | 320 | 100 |
| I-30 | 320 | 100 |
| I-31 | 320 | 100 |
| I-32 | 320 | 100 |
| I-36 | 320 | 100 |
| I-37 | 320 | 100 |
| I-40 | 320 | 100 |
| I-42 | 320 | 80 |
| I-43 | 320 | 100 |
| I-44 | 320 | 90 |
| I-45 | 320 | 100 |
| I-46 | 320 | 100 |
| I-47 | 320 | 100 |
| I-48 | 320 | 100 |
| I-49 | 320 | 100 |
| I-50 | 320 | 100 |
| I-51 | 320 | 100 |
| I-52 | 320 | 100 |
| I-54 | 320 | 100 |
| I-55 | 320 | 100 |
| I-56 | 320 | 100 |
| I-57 | 320 | 100 |
| I-58 | 320 | 100 |
| I-59 | 320 | 100 |
| I-61 | 320 | 100 |
| I-62 | 320 | 100 |
| I-63 | 320 | 100 |
| I-64 | 320 | 100 |
| I-66 | 320 | 100 |
| I-67 | 320 | 100 |
| I-68 | 320 | 100 |
| I-69 | 320 | 100 |
| I-70 | 320 | 100 |
| I-71 | 320 | 100 |
| I-72 | 320 | 100 |
| I-73 | 320 | 100 |
| I-74 | 320 | 100 |
| I-75 | 320 | 90 |
| I-76 | 320 | 100 |
| I-77 | 320 | 100 |
| I-78 | 320 | 100 |
| I-79 | 320 | 100 |
| P-01 | 320 | 100 |
| P-05 | 320 | 100 |
| P-08 | 320 | 100 |
| P-100 | 320 | 100 |
| P-101 | 320 | 100 |
| P-102 | 320 | 100 |
| P-103 | 320 | 100 |
| P-104 | 320 | 100 |
| P-13 | 320 | 100 |
| P-23 | 320 | 100 |
| P-28 | 320 | 100 |
| P-29 | 320 | 100 |
| P-30 | 320 | 100 |
| P-31 | 320 | 100 |
| P-32 | 320 | 100 |
| P-33 | 320 | 100 |
| P-34 | 320 | 100 |
| P-35 | 320 | 100 |
| P-36 | 320 | 90 |
| P-37 | 320 | 100 |
| P-46 | 320 | 100 |
| P-47 | 320 | 100 |
| P-48 | 320 | 100 |
| P-49 | 320 | 100 |
| P-50 | 320 | 100 |
| P-51 | 320 | 100 |
| P-52 | 320 | 100 |
| P-53 | 320 | 100 |
| P-54 | 320 | 100 |
| P-55 | 320 | 100 |
| P-56 | 320 | 100 |
| P-57 | 320 | 100 |
| P-58 | 320 | 100 |
| P-59 | 320 | 100 |
| P-60 | 320 | 100 |
| P-61 | 320 | 100 |
| P-62 | 320 | 100 |
| P-63 | 320 | 90 |
| P-64 | 320 | 90 |
| P-65 | 320 | 100 |
| P-66 | 320 | 100 |
| P-67 | 320 | 100 |
| P-68 | 320 | 100 |
| P-69 | 320 | 100 |
| P-70 | 320 | 100 |
| P-71 | 320 | 100 |
| P-72 | 320 | 100 |
| P-75 | 320 | 100 |
| P-76 | 320 | 100 |
| P-77 | 320 | 100 |
| P-78 | 320 | 100 |
| P-79 | 320 | 100 |
| P-80 | 320 | 100 |
| P-81 | 320 | 100 |
| P-82 | 320 | 100 |
| P-83 | 320 | 100 |
| P-84 | 320 | 100 |
| P-85 | 320 | 100 |
| P-86 | 320 | 100 |
| P-87 | 320 | 100 |
| P-88 | 320 | 100 |
| P-89 | 320 | 100 |
| P-90 | 320 | 100 |
| P-94 | 320 | 100 |
| P-95 | 320 | 100 |
| P-97 | 320 | 100 |
| P-98 | 320 | 100 |
| P-99 | 320 | 100 |

TABLE 8
Post-emergence action at 320 g/ha against LOLRI in %

| Example | Dosage | |
|---|---|---|
| number | [g/ha] | LOLRI |
| I-02 | 320 | 100 |
| I-05 | 320 | 100 |
| I-10 | 320 | 100 |
| I-11 | 320 | 100 |
| I-17 | 320 | 100 |
| I-18 | 320 | 100 |
| I-19 | 320 | 100 |
| I-20 | 320 | 100 |
| I-21 | 320 | 100 |
| I-30 | 320 | 100 |
| I-31 | 320 | 100 |
| I-32 | 320 | 100 |
| I-33 | 320 | 100 |
| I-34 | 320 | 100 |
| I-35 | 320 | 100 |
| I-36 | 320 | 100 |
| I-37 | 320 | 100 |
| I-40 | 320 | 90 |
| I-41 | 320 | 100 |
| I-42 | 320 | 100 |
| I-43 | 320 | 100 |
| I-44 | 320 | 100 |
| I-45 | 320 | 100 |
| I-46 | 320 | 100 |
| I-47 | 320 | 100 |
| I-48 | 320 | 100 |
| I-49 | 320 | 100 |
| I-50 | 320 | 100 |
| I-51 | 320 | 100 |
| I-52 | 320 | 100 |
| I-54 | 320 | 100 |
| I-55 | 320 | 100 |
| I-56 | 320 | 100 |
| I-57 | 320 | 100 |
| I-58 | 320 | 100 |
| I-59 | 320 | 100 |
| I-61 | 320 | 100 |
| I-62 | 320 | 100 |
| I-63 | 320 | 100 |
| I-64 | 320 | 100 |
| I-66 | 320 | 100 |
| I-67 | 320 | 100 |
| I-68 | 320 | 100 |
| I-69 | 320 | 100 |
| I-70 | 320 | 100 |
| I-71 | 320 | 100 |
| I-72 | 320 | 100 |
| I-73 | 320 | 100 |
| I-74 | 320 | 100 |
| I-75 | 320 | 100 |
| I-76 | 320 | 100 |
| I-77 | 320 | 100 |
| I-78 | 320 | 100 |
| I-79 | 320 | 100 |
| P-01 | 320 | 100 |
| P-04 | 320 | 100 |
| P-05 | 320 | 100 |
| P-08 | 320 | 100 |
| P-100 | 320 | 100 |
| P-101 | 320 | 100 |
| P-102 | 320 | 100 |
| P-103 | 320 | 100 |
| P-104 | 320 | 100 |
| P-13 | 320 | 100 |
| P-23 | 320 | 100 |
| P-28 | 320 | 100 |
| P-29 | 320 | 100 |
| P-30 | 320 | 100 |
| P-31 | 320 | 100 |
| P-32 | 320 | 100 |
| P-33 | 320 | 100 |
| P-34 | 320 | 100 |
| P-35 | 320 | 100 |
| P-36 | 320 | 100 |
| P-37 | 320 | 100 |
| P-46 | 320 | 100 |
| P-48 | 320 | 100 |
| P-49 | 320 | 100 |
| P-50 | 320 | 100 |
| P-51 | 320 | 100 |
| P-52 | 320 | 100 |
| P-53 | 320 | 100 |
| P-54 | 320 | 100 |
| P-55 | 320 | 100 |
| P-56 | 320 | 100 |
| P-57 | 320 | 100 |
| P-58 | 320 | 100 |
| P-59 | 320 | 100 |
| P-60 | 320 | 100 |
| P-61 | 320 | 100 |
| P-62 | 320 | 100 |
| P-63 | 320 | 100 |
| P-64 | 320 | 100 |
| P-65 | 320 | 100 |
| P-66 | 320 | 90 |
| P-67 | 320 | 90 |
| P-68 | 320 | 100 |
| P-69 | 320 | 100 |
| P-70 | 320 | 100 |
| P-71 | 320 | 100 |
| P-72 | 320 | 100 |
| P-75 | 320 | 100 |
| P-76 | 320 | 100 |
| P-77 | 320 | 100 |
| P-78 | 320 | 100 |
| P-79 | 320 | 100 |
| P-80 | 320 | 100 |
| P-81 | 320 | 100 |
| P-82 | 320 | 100 |
| P-83 | 320 | 100 |
| P-84 | 320 | 100 |
| P-85 | 320 | 100 |
| P-86 | 320 | 100 |
| P-87 | 320 | 100 |
| P-88 | 320 | 100 |
| P-89 | 320 | 100 |
| P-90 | 320 | 100 |
| P-94 | 320 | 100 |
| P-95 | 320 | 100 |
| P-97 | 320 | 100 |
| P-98 | 320 | 100 |
| P-99 | 320 | 100 |

TABLE 9
Pre-emergence action at 320 g/ha against SETVI in %

| Example | | |
|---|---|---|
| Number | Dosage | SETVI |
| P-04 | 320 | 100 |
| P-13 | 320 | 100 |
| P-23 | 320 | 100 |
| P-08 | 320 | 100 |
| P-05 | 320 | 100 |
| P-01 | 320 | 100 |
| I-10 | 320 | 100 |
| I-11 | 320 | 100 |
| I-05 | 320 | 100 |
| I-17 | 320 | 100 |
| P-29 | 320 | 100 |
| P-28 | 320 | 100 |
| P-33 | 320 | 100 |
| P-32 | 320 | 100 |
| P-30 | 320 | 100 |
| P-31 | 320 | 100 |
| I-18 | 320 | 100 |
| I-19 | 320 | 100 |
| P-37 | 320 | 100 |
| P-36 | 320 | 100 |
| P-35 | 320 | 100 |
| P-34 | 320 | 100 |
| I-21 | 320 | 100 |
| I-20 | 320 | 100 |
| P-45 | 320 | 80 |
| P-100 | 320 | 100 |
| P-99 | 320 | 100 |
| P-101 | 320 | 100 |
| P-103 | 320 | 100 |
| P-104 | 320 | 100 |
| P-102 | 320 | 100 |
| I-79 | 320 | 100 |
| I-78 | 320 | 100 |
| I-74 | 320 | 100 |
| I-75 | 320 | 100 |
| I-76 | 320 | 100 |
| P-46 | 320 | 100 |
| P-60 | 320 | 100 |
| P-50 | 320 | 100 |
| P-47 | 320 | 100 |
| P-55 | 320 | 100 |
| I-77 | 320 | 100 |
| I-02 | 320 | 100 |
| P-49 | 320 | 100 |
| P-48 | 320 | 100 |
| I-31 | 320 | 100 |
| P-58 | 320 | 100 |
| P-57 | 320 | 100 |
| P-59 | 320 | 100 |
| P-56 | 320 | 100 |
| P-51 | 320 | 100 |
| P-52 | 320 | 100 |
| P-53 | 320 | 100 |
| P-54 | 320 | 100 |
| I-30 | 320 | 100 |
| I-32 | 320 | 100 |
| I-34 | 320 | 100 |
| I-36 | 320 | 100 |
| I-33 | 320 | 100 |
| I-35 | 320 | 100 |
| I-37 | 320 | 100 |
| P-65 | 320 | 100 |
| P-66 | 320 | 100 |
| P-67 | 320 | 100 |
| P-64 | 320 | 100 |
| P-63 | 320 | 100 |
| P-62 | 320 | 100 |
| P-69 | 320 | 100 |
| P-61 | 320 | 100 |
| I-41 | 320 | 100 |
| I-40 | 320 | 100 |
| I-42 | 320 | 100 |
| I-43 | 320 | 100 |
| P-68 | 320 | 100 |
| P-70 | 320 | 100 |
| P-71 | 320 | 100 |
| P-72 | 320 | 100 |
| I-45 | 320 | 100 |
| I-44 | 320 | 100 |
| I-46 | 320 | 100 |
| I-47 | 320 | 100 |
| I-48 | 320 | 100 |
| I-49 | 320 | 100 |
| P-79 | 320 | 100 |
| P-80 | 320 | 100 |
| P-75 | 320 | 100 |
| P-76 | 320 | 100 |
| I-51 | 320 | 100 |
| I-50 | 320 | 100 |
| P-78 | 320 | 100 |
| P-77 | 320 | 100 |
| I-54 | 320 | 100 |
| I-55 | 320 | 100 |
| I-52 | 320 | 100 |
| P-95 | 320 | 100 |
| P-89 | 320 | 100 |
| P-90 | 320 | 100 |
| P-94 | 320 | 100 |
| I-64 | 320 | 100 |
| I-63 | 320 | 100 |
| I-66 | 320 | 100 |
| I-69 | 320 | 100 |
| I-61 | 320 | 100 |
| I-62 | 320 | 100 |
| P-82 | 320 | 100 |
| P-84 | 320 | 100 |
| P-83 | 320 | 100 |
| P-81 | 320 | 100 |
| I-57 | 320 | 100 |
| I-56 | 320 | 100 |
| P-87 | 320 | 100 |
| P-86 | 320 | 100 |
| I-58 | 320 | 100 |
| P-85 | 320 | 100 |
| P-88 | 320 | 100 |
| I-59 | 320 | 100 |
| I-68 | 320 | 100 |
| I-71 | 320 | 100 |
| I-70 | 320 | 100 |
| I-67 | 320 | 100 |
| P-97 | 320 | 100 |
| P-98 | 320 | 100 |
| I-73 | 320 | 100 |
| I-72 | 320 | 100 |

TABLE 10
Pre-emergence action at 320 g/ha against ABUTH in %

| Example | Dosage | |
|---|---|---|
| number | [g/ha] | ABUTH |
| I-02 | 320 | 100 |
| I-05 | 320 | 80 |
| I-17 | 320 | 90 |
| I-18 | 320 | 80 |
| I-44 | 320 | 80 |
| I-50 | 320 | 100 |
| I-68 | 320 | 90 |
| I-72 | 320 | 100 |
| I-73 | 320 | 100 |
| P-08 | 320 | 80 |
| P-100 | 320 | 80 |
| P-28 | 320 | 90 |
| P-29 | 320 | 80 |
| P-30 | 320 | 80 |
| P-31 | 320 | 90 |
| P-32 | 320 | 80 |
| P-34 | 320 | 90 |
| P-51 | 320 | 80 |
| P-58 | 320 | 80 |
| P-70 | 320 | 80 |
| P-75 | 320 | 80 |
| P-87 | 320 | 80 |
| P-88 | 320 | 80 |
| P-97 | 320 | 100 |
| P-98 | 320 | 90 |

TABLE 11
Pre-emergence action at 320 g/ha against AMARE in %

| Example | Dosage | |
|---|---|---|
| number | [g/ha] | AMARE |
| I-02 | 320 | 100 |
| I-17 | 320 | 100 |
| I-18 | 320 | 100 |
| I-19 | 320 | 100 |
| I-21 | 320 | 100 |
| I-30 | 320 | 100 |
| I-35 | 320 | 90 |
| I-36 | 320 | 90 |
| I-39 | 320 | 80 |
| I-44 | 320 | 100 |
| I-46 | 320 | 100 |
| I-48 | 320 | 90 |
| I-50 | 320 | 80 |
| I-51 | 320 | 100 |
| I-52 | 320 | 80 |
| I-55 | 320 | 80 |
| I-58 | 320 | 100 |
| I-59 | 320 | 100 |
| I-61 | 320 | 100 |
| I-62 | 320 | 90 |
| I-68 | 320 | 100 |
| I-73 | 320 | 100 |
| I-75 | 320 | 80 |
| I-77 | 320 | 100 |
| I-78 | 320 | 100 |
| I-79 | 320 | 100 |
| P-01 | 320 | 100 |
| P-04 | 320 | 80 |
| P-05 | 320 | 100 |
| P-100 | 320 | 100 |
| P-101 | 320 | 100 |
| P-102 | 320 | 90 |
| P-103 | 320 | 100 |
| P-104 | 320 | 100 |
| P-13 | 320 | 90 |
| P-23 | 320 | 90 |
| P-28 | 320 | 100 |
| P-29 | 320 | 100 |
| P-30 | 320 | 100 |
| P-31 | 320 | 100 |
| P-32 | 320 | 90 |
| P-33 | 320 | 100 |
| P-35 | 320 | 100 |
| P-37 | 320 | 100 |
| P-46 | 320 | 100 |
| P-47 | 320 | 100 |
| P-50 | 320 | 100 |
| P-51 | 320 | 90 |
| P-52 | 320 | 80 |
| P-54 | 320 | 100 |
| P-56 | 320 | 80 |
| P-57 | 320 | 80 |
| P-58 | 320 | 90 |
| P-60 | 320 | 100 |
| P-67 | 320 | 80 |
| P-75 | 320 | 100 |
| P-76 | 320 | 100 |
| P-79 | 320 | 80 |
| P-80 | 320 | 90 |
| P-81 | 320 | 100 |
| P-82 | 320 | 90 |
| P-86 | 320 | 100 |
| P-97 | 320 | 100 |
| P-98 | 320 | 100 |
| P-99 | 320 | 100 |

TABLE 12
Pre-emergence action at 320 g/ha against MATIN in %

| Example | Dosage | |
|---|---|---|
| number | [g/ha] | MATIN |
| I-02 | 320 | 90 |
| I-17 | 320 | 90 |
| I-19 | 320 | 80 |
| I-30 | 320 | 90 |
| I-42 | 320 | 80 |
| I-44 | 320 | 80 |
| I-46 | 320 | 90 |
| I-48 | 320 | 80 |
| I-50 | 320 | 80 |
| I-51 | 320 | 100 |
| I-58 | 320 | 100 |
| I-59 | 320 | 80 |
| I-61 | 320 | 80 |
| I-67 | 320 | 80 |
| I-68 | 320 | 100 |
| I-70 | 320 | 80 |
| I-71 | 320 | 90 |
| I-72 | 320 | 100 |
| I-73 | 320 | 90 |
| I-75 | 320 | 90 |
| I-77 | 320 | 80 |
| I-78 | 320 | 80 |
| I-79 | 320 | 90 |
| P-01 | 320 | 80 |
| P-04 | 320 | 90 |
| P-05 | 320 | 90 |
| P-08 | 320 | 80 |
| P-100 | 320 | 90 |
| P-101 | 320 | 80 |
| P-103 | 320 | 90 |
| P-13 | 320 | 80 |
| P-28 | 320 | 90 |
| P-29 | 320 | 90 |
| P-32 | 320 | 80 |
| P-49 | 320 | 80 |
| P-53 | 320 | 90 |
| P-55 | 320 | 90 |
| P-56 | 320 | 80 |
| P-60 | 320 | 90 |
| P-75 | 320 | 90 |
| P-80 | 320 | 90 |
| P-81 | 320 | 80 |
| P-84 | 320 | 80 |
| P-86 | 320 | 80 |
| P-97 | 320 | 100 |
| P-98 | 320 | 90 |

TABLE 13
Pre-emergence action at 320 g/ha against PHBPU in %

| Example | Dosage | |
|---|---|---|
| number | [g/ha] | PHBPU |
| I-61 | 320 | 80 |
| P-13 | 320 | 100 |

TABLE 14
Pre-emergence action at 320 g/ha against POLCO in %

| Example | Dosage | |
|---|---|---|
| number | [g/ha] | POLCO |
| I-02 | 320 | 100 |
| I-05 | 320 | 80 |
| I-10 | 320 | 80 |
| I-18 | 320 | 100 |
| I-19 | 320 | 100 |
| I-44 | 320 | 100 |
| I-46 | 320 | 90 |
| I-48 | 320 | 90 |
| I-50 | 320 | 90 |
| I-51 | 320 | 80 |
| I-61 | 320 | 90 |
| I-69 | 320 | 100 |
| I-70 | 320 | 80 |
| I-79 | 320 | 90 |
| P-08 | 320 | 100 |
| P-13 | 320 | 80 |
| P-28 | 320 | 80 |
| P-29 | 320 | 90 |
| P-30 | 320 | 80 |
| P-31 | 320 | 90 |
| P-32 | 320 | 80 |
| P-33 | 320 | 100 |
| P-51 | 320 | 90 |
| P-52 | 320 | 80 |
| P-54 | 320 | 80 |
| P-60 | 320 | 80 |
| P-70 | 320 | 80 |
| P-71 | 320 | 90 |
| P-72 | 320 | 100 |
| P-75 | 320 | 80 |
| P-76 | 320 | 100 |
| P-80 | 320 | 90 |
| P-81 | 320 | 100 |
| P-82 | 320 | 100 |
| P-86 | 320 | 90 |
| P-87 | 320 | 80 |
| P-89 | 320 | 100 |

TABLE 15
Pre-emergence action at 320 g/ha against VIOTR in %

| Example | Dosage | |
|---|---|---|
| number | [g/ha] | VIOTR |
| I-02 | 320 | 100 |
| I-05 | 320 | 90 |
| I-10 | 320 | 100 |
| I-11 | 320 | 90 |
| I-17 | 320 | 100 |
| I-18 | 320 | 100 |
| I-19 | 320 | 100 |
| I-20 | 320 | 100 |
| I-21 | 320 | 100 |
| I-30 | 320 | 100 |
| I-31 | 320 | 80 |
| I-32 | 320 | 100 |
| I-33 | 320 | 100 |
| I-35 | 320 | 100 |
| I-36 | 320 | 80 |
| I-42 | 320 | 80 |
| I-44 | 320 | 100 |
| I-45 | 320 | 80 |
| I-46 | 320 | 100 |
| I-47 | 320 | 100 |
| I-48 | 320 | 100 |
| I-49 | 320 | 100 |
| I-50 | 320 | 100 |
| I-51 | 320 | 100 |
| I-52 | 320 | 80 |
| I-58 | 320 | 90 |
| I-59 | 320 | 100 |
| I-61 | 320 | 100 |
| I-62 | 320 | 100 |
| I-67 | 320 | 100 |
| I-68 | 320 | 100 |
| I-69 | 320 | 90 |
| I-70 | 320 | 100 |
| I-71 | 320 | 100 |
| I-72 | 320 | 100 |
| I-73 | 320 | 100 |
| I-76 | 320 | 90 |
| I-78 | 320 | 100 |
| I-79 | 320 | 100 |
| P-01 | 320 | 100 |
| P-04 | 320 | 100 |
| P-05 | 320 | 100 |
| P-08 | 320 | 90 |
| P-100 | 320 | 100 |
| P-101 | 320 | 80 |
| P-13 | 320 | 100 |
| P-23 | 320 | 100 |
| P-28 | 320 | 100 |
| P-29 | 320 | 100 |
| P-30 | 320 | 100 |
| P-31 | 320 | 100 |
| P-32 | 320 | 100 |
| P-33 | 320 | 100 |
| P-34 | 320 | 100 |
| P-35 | 320 | 100 |
| P-36 | 320 | 100 |
| P-37 | 320 | 100 |
| P-43 | 320 | 100 |
| P-46 | 320 | 100 |
| P-47 | 320 | 100 |
| P-48 | 320 | 100 |
| P-49 | 320 | 100 |
| P-50 | 320 | 100 |
| P-51 | 320 | 100 |
| P-52 | 320 | 90 |
| P-53 | 320 | 80 |
| P-54 | 320 | 100 |
| P-55 | 320 | 100 |
| P-56 | 320 | 100 |
| P-57 | 320 | 100 |
| P-58 | 320 | 100 |
| P-59 | 320 | 100 |
| P-60 | 320 | 100 |
| P-65 | 320 | 80 |
| P-69 | 320 | 80 |
| P-70 | 320 | 80 |
| P-71 | 320 | 100 |
| P-72 | 320 | 100 |
| P-75 | 320 | 100 |
| P-76 | 320 | 100 |
| P-77 | 320 | 100 |
| P-79 | 320 | 100 |
| P-80 | 320 | 100 |
| P-81 | 320 | 100 |
| P-82 | 320 | 90 |
| P-83 | 320 | 90 |
| P-84 | 320 | 80 |
| P-85 | 320 | 100 |
| P-86 | 320 | 100 |
| P-87 | 320 | 90 |
| P-88 | 320 | 100 |
| P-89 | 320 | 80 |
| P-97 | 320 | 90 |
| P-98 | 320 | 100 |
| P-99 | 320 | 100 |

TABLE 16
Pre-emergence action at 320 g/ha against VERPE in %

| Example | Dosage | |
|---|---|---|
| number | [g/ha] | VERPE |
| I-02 | 320 | 90 |
| I-10 | 320 | 100 |
| I-11 | 320 | 100 |
| I-17 | 320 | 90 |
| I-24 | 320 | 80 |
| I-44 | 320 | 80 |
| I-51 | 320 | 100 |
| I-62 | 320 | 100 |
| I-69 | 320 | 80 |
| I-71 | 320 | 90 |
| I-73 | 320 | 90 |
| I-78 | 320 | 90 |
| I-79 | 320 | 80 |
| P-01 | 320 | 100 |
| P-04 | 320 | 90 |
| P-05 | 320 | 80 |
| P-08 | 320 | 90 |
| P-100 | 320 | 90 |
| P-103 | 320 | 90 |
| P-23 | 320 | 90 |
| P-28 | 320 | 100 |
| P-29 | 320 | 100 |
| P-30 | 320 | 80 |
| P-31 | 320 | 90 |
| P-32 | 320 | 90 |
| P-33 | 320 | 80 |
| P-34 | 320 | 80 |
| P-35 | 320 | 80 |
| P-36 | 320 | 80 |
| P-37 | 320 | 80 |
| P-48 | 320 | 80 |
| P-49 | 320 | 80 |
| P-50 | 320 | 80 |
| P-54 | 320 | 80 |
| P-55 | 320 | 80 |
| P-60 | 320 | 80 |
| P-61 | 320 | 90 |
| P-67 | 320 | 80 |
| P-69 | 320 | 90 |
| P-70 | 320 | 90 |
| P-71 | 320 | 90 |
| P-72 | 320 | 80 |
| P-75 | 320 | 90 |
| P-76 | 320 | 100 |
| P-77 | 320 | 80 |
| P-79 | 320 | 90 |
| P-80 | 320 | 100 |
| P-81 | 320 | 90 |
| P-83 | 320 | 80 |
| P-85 | 320 | 90 |
| P-86 | 320 | 100 |
| P-87 | 320 | 80 |
| P-88 | 320 | 100 |
| P-97 | 320 | 100 |
| P-98 | 320 | 100 |
| P-99 | 320 | 100 |

For example, compound nos. P-70, P-81, P-05, P-77, P-76, I-10, I-2 and I-11 in tables 4-18, at an application rate of 320 g/ha, each show 90-100% efficacy against Alopecurus myosuroides, Avena fatua, Digitaria sanguinalis, Echinochloa crus-galli, Lolium rigidum, and Setaria viridis.

The compounds of the invention are therefore suitable for control of unwanted plant growth by the pre-emergence method.

2. Post-Emergence Herbicidal Effect and Crop Plant Compatibility

Seeds of monocotyledonous and dicotyledonous weed and crop plants are laid out in sandy loam soil in wood-fiber pots, covered with soil and cultivated in a greenhouse under good growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants as aqueous suspension or emulsion at a water application rate equating to 600 to 800 l/ha with addition of 0.2% wetting agent. After the test plants have been left to stand in the greenhouse under optimal growth conditions for about 3 weeks, the action of the preparations is assessed visually in comparison to untreated controls (herbicidal action in percent (%): 100% activity=the plants have died, 0% activity=like control plants).

As the results from tables 5 and 6 show, compounds of the invention have good herbicidal post-emergence efficacy against a broad spectrum of weed grasses and weeds.

TABLE 17
Post-emergence action at 80 g/ha against ALOMY in %

| Example | Dosage | |
|---|---|---|
| number | [g/ha] | ALOMY |
| I-01 | 80 | 100 |
| I-02 | 80 | 100 |
| I-03 | 80 | 100 |
| I-04 | 80 | 100 |
| I-05 | 80 | 100 |
| I-06 | 80 | 100 |
| I-07 | 80 | 100 |
| I-08 | 80 | 80 |
| I-09 | 80 | 100 |
| I-10 | 80 | 100 |
| I-11 | 80 | 100 |
| I-12 | 80 | 100 |
| I-13 | 80 | 100 |
| I-14 | 80 | 90 |
| I-15 | 80 | 100 |
| I-16 | 80 | 100 |
| I-17 | 80 | 100 |
| I-18 | 80 | 100 |
| I-19 | 80 | 100 |
| I-20 | 80 | 100 |
| I-21 | 80 | 100 |
| I-30 | 80 | 90 |
| I-31 | 80 | 100 |
| I-32 | 80 | 100 |
| I-33 | 80 | 100 |
| I-34 | 80 | 100 |
| I-35 | 80 | 100 |
| I-36 | 80 | 100 |
| I-37 | 80 | 100 |
| I-40 | 80 | 100 |
| I-41 | 80 | 100 |
| I-42 | 80 | 90 |
| I-43 | 80 | 100 |
| I-44 | 80 | 100 |
| I-45 | 80 | 90 |
| I-47 | 80 | 100 |
| I-48 | 80 | 90 |
| I-49 | 80 | 90 |
| I-50 | 80 | 100 |
| I-51 | 80 | 100 |
| I-52 | 80 | 100 |
| I-54 | 80 | 100 |
| I-55 | 80 | 100 |
| I-56 | 80 | 90 |
| I-58 | 80 | 100 |
| I-59 | 80 | 100 |
| I-61 | 80 | 100 |
| I-62 | 80 | 100 |
| I-63 | 80 | 100 |
| I-64 | 80 | 90 |
| I-66 | 80 | 100 |
| I-67 | 80 | 100 |
| I-68 | 80 | 100 |
| I-69 | 80 | 100 |
| I-70 | 80 | 90 |
| I-71 | 80 | 90 |
| I-72 | 80 | 90 |
| I-73 | 80 | 100 |
| I-74 | 80 | 90 |
| I-75 | 80 | 90 |
| I-76 | 80 | 90 |
| I-77 | 80 | 100 |
| I-78 | 80 | 100 |
| I-79 | 80 | 90 |
| P-01 | 80 | 100 |
| P-02 | 80 | 100 |
| P-03 | 80 | 100 |
| P-04 | 80 | 100 |
| P-05 | 80 | 100 |
| P-06 | 80 | 100 |
| P-07 | 80 | 100 |
| P-08 | 80 | 100 |
| P-09 | 80 | 100 |
| P-100 | 80 | 100 |
| P-101 | 80 | 100 |
| P-102 | 80 | 90 |
| P-103 | 80 | 100 |
| P-104 | 80 | 100 |
| P-11 | 80 | 80 |
| P-12 | 80 | 100 |
| P-13 | 80 | 100 |
| P-14 | 80 | 100 |
| P-15 | 80 | 100 |
| P-16 | 80 | 100 |
| P-17 | 80 | 100 |
| P-18 | 80 | 100 |
| P-19 | 80 | 80 |
| P-20 | 80 | 100 |
| P-21 | 80 | 100 |
| P-22 | 80 | 100 |
| P-23 | 80 | 100 |
| P-24 | 80 | 100 |
| P-25 | 80 | 100 |
| P-26 | 80 | 100 |
| P-27 | 80 | 90 |
| P-28 | 80 | 100 |
| P-29 | 80 | 100 |
| P-30 | 80 | 100 |
| P-31 | 80 | 90 |
| P-32 | 80 | 100 |
| P-33 | 80 | 100 |
| P-34 | 80 | 100 |
| P-35 | 80 | 100 |
| P-36 | 80 | 100 |
| P-37 | 80 | 100 |
| P-46 | 80 | 100 |
| P-47 | 80 | 100 |
| P-48 | 80 | 100 |
| P-49 | 80 | 100 |
| P-50 | 80 | 100 |
| P-51 | 80 | 100 |
| P-52 | 80 | 100 |
| P-53 | 80 | 100 |
| P-54 | 80 | 100 |
| P-55 | 80 | 100 |
| P-56 | 80 | 100 |
| P-57 | 80 | 100 |
| P-58 | 80 | 100 |
| P-59 | 80 | 100 |
| P-60 | 80 | 100 |
| P-61 | 80 | 90 |
| P-62 | 80 | 90 |
| P-63 | 80 | 100 |
| P-64 | 80 | 100 |
| P-65 | 80 | 90 |
| P-66 | 80 | 90 |
| P-67 | 80 | 100 |
| P-68 | 80 | 100 |
| P-69 | 80 | 100 |
| P-70 | 80 | 100 |
| P-71 | 80 | 80 |
| P-72 | 80 | 80 |
| P-75 | 80 | 100 |
| P-76 | 80 | 100 |
| P-77 | 80 | 90 |
| P-78 | 80 | 100 |
| P-79 | 80 | 100 |
| P-80 | 80 | 100 |
| P-81 | 80 | 100 |
| P-82 | 80 | 100 |
| P-83 | 80 | 100 |
| P-85 | 80 | 80 |
| P-86 | 80 | 90 |
| P-87 | 80 | 90 |
| P-88 | 80 | 100 |
| P-89 | 80 | 100 |
| P-90 | 80 | 100 |
| P-95 | 80 | 100 |
| P-97 | 80 | 100 |
| P-98 | 80 | 100 |
| P-99 | 80 | 100 |

TABLE 18
Post-emergence action at 80 g/ha against AVEFA in %

| Example | Dosage | |
|---|---|---|
| number | [g/ha] | AVEFA |
| I-01 | 80 | 90 |
| I-02 | 80 | 90 |
| I-03 | 80 | 100 |
| I-04 | 80 | 100 |
| I-06 | 80 | 100 |
| I-07 | 80 | 100 |
| I-08 | 80 | 90 |
| I-09 | 80 | 100 |
| I-12 | 80 | 100 |
| I-13 | 80 | 90 |
| I-14 | 80 | 90 |
| I-15 | 80 | 100 |
| I-16 | 80 | 100 |
| I-17 | 80 | 90 |
| I-18 | 80 | 100 |
| I-19 | 80 | 90 |
| I-30 | 80 | 80 |
| I-31 | 80 | 100 |
| I-32 | 80 | 100 |
| I-33 | 80 | 100 |
| I-34 | 80 | 90 |
| I-35 | 80 | 80 |
| I-36 | 80 | 100 |
| I-37 | 80 | 100 |
| I-40 | 80 | 90 |
| I-41 | 80 | 90 |
| I-42 | 80 | 90 |
| I-43 | 80 | 90 |
| I-44 | 80 | 80 |
| I-45 | 80 | 80 |
| I-47 | 80 | 90 |
| I-50 | 80 | 80 |
| I-51 | 80 | 100 |
| I-52 | 80 | 90 |
| I-55 | 80 | 90 |
| I-56 | 80 | 90 |
| I-59 | 80 | 90 |
| I-63 | 80 | 80 |
| I-64 | 80 | 80 |
| I-66 | 80 | 80 |
| I-71 | 80 | 80 |
| I-72 | 80 | 100 |
| I-73 | 80 | 100 |
| I-74 | 80 | 80 |
| I-75 | 80 | 90 |
| I-76 | 80 | 90 |
| I-77 | 80 | 90 |
| I-78 | 80 | 80 |
| I-79 | 80 | 90 |
| P-02 | 80 | 100 |
| P-03 | 80 | 100 |
| P-06 | 80 | 100 |
| P-07 | 80 | 100 |
| P-09 | 80 | 100 |
| P-100 | 80 | 80 |
| P-101 | 80 | 80 |
| P-102 | 80 | 80 |
| P-103 | 80 | 90 |
| P-104 | 80 | 90 |
| P-11 | 80 | 100 |
| P-12 | 80 | 100 |
| P-14 | 80 | 100 |
| P-15 | 80 | 100 |
| P-16 | 80 | 100 |
| P-17 | 80 | 100 |
| P-18 | 80 | 100 |
| P-19 | 80 | 100 |
| P-20 | 80 | 100 |
| P-21 | 80 | 100 |
| P-22 | 80 | 100 |
| P-24 | 80 | 100 |
| P-25 | 80 | 100 |
| P-26 | 80 | 100 |
| P-27 | 80 | 90 |
| P-28 | 80 | 90 |
| P-29 | 80 | 90 |
| P-30 | 80 | 90 |
| P-31 | 80 | 100 |
| P-32 | 80 | 90 |
| P-33 | 80 | 90 |
| P-34 | 80 | 90 |
| P-35 | 80 | 80 |
| P-37 | 80 | 90 |
| P-46 | 80 | 90 |
| P-47 | 80 | 90 |
| P-48 | 80 | 90 |
| P-49 | 80 | 90 |
| P-50 | 80 | 90 |
| P-51 | 80 | 100 |
| P-52 | 80 | 90 |
| P-53 | 80 | 90 |
| P-54 | 80 | 100 |
| P-55 | 80 | 100 |
| P-56 | 80 | 90 |
| P-57 | 80 | 90 |
| P-58 | 80 | 90 |
| P-59 | 80 | 90 |
| P-60 | 80 | 100 |
| P-61 | 80 | 80 |
| P-62 | 80 | 90 |
| P-63 | 80 | 80 |
| P-64 | 80 | 80 |
| P-65 | 80 | 90 |
| P-66 | 80 | 90 |
| P-67 | 80 | 90 |
| P-68 | 80 | 90 |
| P-69 | 80 | 90 |
| P-71 | 80 | 80 |
| P-75 | 80 | 90 |
| P-76 | 80 | 90 |
| P-79 | 80 | 90 |
| P-80 | 80 | 90 |
| P-81 | 80 | 80 |
| P-83 | 80 | 90 |
| P-84 | 80 | 80 |
| P-87 | 80 | 80 |
| P-90 | 80 | 80 |
| P-95 | 80 | 80 |
| P-97 | 80 | 100 |
| P-98 | 80 | 90 |
| P-99 | 80 | 80 |

TABLE 19
Post-emergence action at 80 g/ha against DIGSA in %

| Example | Dosage | |
|---|---|---|
| number | [g/ha] | DIGSA |
| I-01 | 80 | 90 |
| I-02 | 80 | 90 |
| I-03 | 80 | 100 |
| I-04 | 80 | 100 |
| I-05 | 80 | 100 |
| I-06 | 80 | 100 |
| I-07 | 80 | 100 |
| I-08 | 80 | 80 |
| I-09 | 80 | 100 |
| I-10 | 80 | 100 |
| I-11 | 80 | 100 |
| I-12 | 80 | 100 |
| I-13 | 80 | 100 |
| I-14 | 80 | 90 |
| I-15 | 80 | 100 |
| I-16 | 80 | 100 |
| I-17 | 80 | 100 |
| I-18 | 80 | 100 |
| I-19 | 80 | 100 |
| I-20 | 80 | 90 |
| I-21 | 80 | 100 |
| I-30 | 80 | 100 |
| I-31 | 80 | 100 |
| I-32 | 80 | 100 |
| I-33 | 80 | 100 |
| I-34 | 80 | 100 |
| I-35 | 80 | 100 |
| I-36 | 80 | 100 |
| I-37 | 80 | 100 |
| I-40 | 80 | 100 |
| I-41 | 80 | 100 |
| I-42 | 80 | 100 |
| I-44 | 80 | 100 |
| I-45 | 80 | 90 |
| I-46 | 80 | 90 |
| I-51 | 80 | 100 |
| I-56 | 80 | 90 |
| I-57 | 80 | 90 |
| I-58 | 80 | 90 |
| I-59 | 80 | 100 |
| I-61 | 80 | 90 |
| I-62 | 80 | 90 |
| I-63 | 80 | 90 |
| I-64 | 80 | 80 |
| I-66 | 80 | 90 |
| I-67 | 80 | 90 |
| I-68 | 80 | 100 |
| I-69 | 80 | 90 |
| I-70 | 80 | 90 |
| I-71 | 80 | 90 |
| I-72 | 80 | 100 |
| I-73 | 80 | 100 |
| I-74 | 80 | 90 |
| I-75 | 80 | 90 |
| I-76 | 80 | 90 |
| I-77 | 80 | 90 |
| I-78 | 80 | 90 |
| I-79 | 80 | 100 |
| P-01 | 80 | 100 |
| P-02 | 80 | 100 |
| P-03 | 80 | 100 |
| P-04 | 80 | 100 |
| P-05 | 80 | 100 |
| P-06 | 80 | 100 |
| P-07 | 80 | 100 |
| P-08 | 80 | 100 |
| P-09 | 80 | 100 |
| P-100 | 80 | 90 |
| P-101 | 80 | 90 |
| P-102 | 80 | 90 |
| P-103 | 80 | 100 |
| P-104 | 80 | 90 |
| P-11 | 80 | 100 |
| P-12 | 80 | 100 |
| P-13 | 80 | 100 |
| P-14 | 80 | 100 |
| P-15 | 80 | 100 |
| P-16 | 80 | 100 |
| P-17 | 80 | 100 |
| P-18 | 80 | 100 |
| P-19 | 80 | 90 |
| P-20 | 80 | 100 |
| P-21 | 80 | 100 |
| P-22 | 80 | 100 |
| P-23 | 80 | 100 |
| P-24 | 80 | 100 |
| P-25 | 80 | 100 |
| P-26 | 80 | 100 |
| P-27 | 80 | 90 |
| P-28 | 80 | 100 |
| P-29 | 80 | 100 |
| P-30 | 80 | 100 |
| P-31 | 80 | 100 |
| P-32 | 80 | 100 |
| P-33 | 80 | 100 |
| P-34 | 80 | 100 |
| P-35 | 80 | 100 |
| P-36 | 80 | 90 |
| P-37 | 80 | 100 |
| P-46 | 80 | 100 |
| P-47 | 80 | 100 |
| P-48 | 80 | 100 |
| P-49 | 80 | 90 |
| P-50 | 80 | 90 |
| P-51 | 80 | 100 |
| P-52 | 80 | 100 |
| P-53 | 80 | 100 |
| P-54 | 80 | 100 |
| P-55 | 80 | 100 |
| P-56 | 80 | 100 |
| P-57 | 80 | 100 |
| P-58 | 80 | 100 |
| P-59 | 80 | 100 |
| P-60 | 80 | 90 |
| P-61 | 80 | 100 |
| P-62 | 80 | 100 |
| P-63 | 80 | 100 |
| P-64 | 80 | 100 |
| P-65 | 80 | 100 |
| P-66 | 80 | 100 |
| P-68 | 80 | 90 |
| P-69 | 80 | 80 |
| P-70 | 80 | 100 |
| P-71 | 80 | 90 |
| P-72 | 80 | 90 |
| P-75 | 80 | 90 |
| P-76 | 80 | 90 |
| P-77 | 80 | 80 |
| P-78 | 80 | 90 |
| P-79 | 80 | 90 |
| P-80 | 80 | 90 |
| P-81 | 80 | 90 |
| P-82 | 80 | 90 |
| P-83 | 80 | 100 |
| P-84 | 80 | 90 |
| P-85 | 80 | 90 |
| P-86 | 80 | 90 |
| P-87 | 80 | 100 |
| P-88 | 80 | 90 |
| P-89 | 80 | 90 |
| P-90 | 80 | 90 |
| P-94 | 80 | 80 |
| P-95 | 80 | 80 |
| P-97 | 80 | 100 |
| P-98 | 80 | 100 |
| P-99 | 80 | 90 |

TABLE 20
Post-emergence action at 80 g/ha against ECHCG in %

| Example | Dosage | |
|---|---|---|
| number | [g/ha] | ECHCG |
| I-01 | 80 | 90 |
| I-02 | 80 | 100 |
| I-03 | 80 | 100 |
| I-04 | 80 | 100 |
| I-05 | 80 | 100 |
| I-06 | 80 | 100 |
| I-07 | 80 | 100 |
| I-09 | 80 | 100 |
| I-10 | 80 | 100 |
| I-11 | 80 | 100 |
| I-12 | 80 | 100 |
| I-13 | 80 | 100 |
| I-14 | 80 | 90 |
| I-15 | 80 | 100 |
| I-16 | 80 | 100 |
| I-17 | 80 | 100 |
| I-18 | 80 | 100 |
| I-19 | 80 | 100 |
| I-20 | 80 | 100 |
| I-21 | 80 | 100 |
| I-30 | 80 | 100 |
| I-31 | 80 | 100 |
| I-32 | 80 | 100 |
| I-33 | 80 | 100 |
| I-34 | 80 | 100 |
| I-35 | 80 | 100 |
| I-36 | 80 | 100 |
| I-37 | 80 | 100 |
| I-40 | 80 | 100 |
| I-41 | 80 | 100 |
| I-42 | 80 | 100 |
| I-43 | 80 | 100 |
| I-44 | 80 | 100 |
| I-45 | 80 | 100 |
| I-46 | 80 | 100 |
| I-47 | 80 | 100 |
| I-48 | 80 | 100 |
| I-49 | 80 | 100 |
| I-50 | 80 | 100 |
| I-51 | 80 | 100 |
| I-52 | 80 | 100 |
| I-54 | 80 | 100 |
| I-55 | 80 | 100 |
| I-56 | 80 | 100 |
| I-57 | 80 | 100 |
| I-58 | 80 | 100 |
| I-59 | 80 | 100 |
| I-61 | 80 | 100 |
| I-62 | 80 | 100 |
| I-63 | 80 | 100 |
| I-64 | 80 | 100 |
| I-66 | 80 | 100 |
| I-67 | 80 | 90 |
| I-68 | 80 | 100 |
| I-69 | 80 | 100 |
| I-70 | 80 | 100 |
| I-71 | 80 | 90 |
| I-72 | 80 | 100 |
| I-73 | 80 | 100 |
| I-74 | 80 | 100 |
| I-75 | 80 | 100 |
| I-76 | 80 | 100 |
| I-77 | 80 | 100 |
| I-78 | 80 | 90 |
| I-79 | 80 | 100 |
| P-01 | 80 | 100 |
| P-02 | 80 | 100 |
| P-03 | 80 | 100 |
| P-04 | 80 | 100 |
| P-05 | 80 | 100 |
| P-06 | 80 | 100 |
| P-07 | 80 | 100 |
| P-08 | 80 | 100 |
| P-09 | 80 | 100 |
| P-100 | 80 | 100 |
| P-101 | 80 | 100 |
| P-102 | 80 | 80 |
| P-103 | 80 | 100 |
| P-104 | 80 | 100 |
| P-12 | 80 | 100 |
| P-13 | 80 | 100 |
| P-14 | 80 | 100 |
| P-15 | 80 | 100 |
| P-16 | 80 | 100 |
| P-17 | 80 | 100 |
| P-18 | 80 | 100 |
| P-19 | 80 | 100 |
| P-20 | 80 | 100 |
| P-21 | 80 | 100 |
| P-22 | 80 | 100 |
| P-23 | 80 | 100 |
| P-24 | 80 | 100 |
| P-25 | 80 | 100 |
| P-26 | 80 | 100 |
| P-27 | 80 | 90 |
| P-28 | 80 | 100 |
| P-29 | 80 | 100 |
| P-30 | 80 | 100 |
| P-31 | 80 | 100 |
| P-32 | 80 | 100 |
| P-34 | 80 | 100 |
| P-35 | 80 | 90 |
| P-36 | 80 | 90 |
| P-37 | 80 | 100 |
| P-46 | 80 | 100 |
| P-47 | 80 | 100 |
| P-48 | 80 | 100 |
| P-49 | 80 | 100 |
| P-50 | 80 | 100 |
| P-51 | 80 | 100 |
| P-52 | 80 | 100 |
| P-53 | 80 | 100 |
| P-54 | 80 | 100 |
| P-55 | 80 | 100 |
| P-56 | 80 | 100 |
| P-57 | 80 | 100 |
| P-58 | 80 | 100 |
| P-59 | 80 | 100 |
| P-60 | 80 | 100 |
| P-61 | 80 | 100 |
| P-62 | 80 | 100 |
| P-63 | 80 | 100 |
| P-64 | 80 | 100 |
| P-65 | 80 | 90 |
| P-66 | 80 | 100 |
| P-67 | 80 | 100 |
| P-68 | 80 | 100 |
| P-69 | 80 | 100 |
| P-70 | 80 | 100 |
| P-71 | 80 | 100 |
| P-72 | 80 | 100 |
| P-75 | 80 | 100 |
| P-76 | 80 | 100 |
| P-77 | 80 | 100 |
| P-78 | 80 | 100 |
| P-79 | 80 | 100 |
| P-80 | 80 | 100 |
| P-81 | 80 | 100 |
| P-82 | 80 | 100 |
| P-83 | 80 | 100 |
| P-84 | 80 | 100 |
| P-85 | 80 | 100 |
| P-86 | 80 | 100 |
| P-87 | 80 | 100 |
| P-88 | 80 | 100 |
| P-89 | 80 | 100 |
| P-90 | 80 | 100 |
| P-94 | 80 | 100 |
| P-95 | 80 | 100 |
| P-97 | 80 | 100 |
| P-98 | 80 | 100 |
| P-99 | 80 | 100 |

TABLE 21
Post-emergence action at 80 g/ha against LOLRI in %

| Example | Dosage | |
|---|---|---|
| number | [g/ha] | LOLRI |
| I-01 | 80 | 100 |
| I-02 | 80 | 100 |
| I-03 | 80 | 100 |
| I-04 | 80 | 100 |
| I-05 | 80 | 100 |
| I-06 | 80 | 100 |
| I-07 | 80 | 100 |
| I-08 | 80 | 90 |
| I-09 | 80 | 100 |
| I-10 | 80 | 100 |
| I-11 | 80 | 100 |
| I-12 | 80 | 100 |
| I-13 | 80 | 90 |
| I-14 | 80 | 90 |
| I-15 | 80 | 100 |
| I-16 | 80 | 100 |
| I-17 | 80 | 100 |
| I-18 | 80 | 100 |
| I-19 | 80 | 100 |
| I-20 | 80 | 100 |
| I-30 | 80 | 100 |
| I-31 | 80 | 100 |
| I-32 | 80 | 100 |
| I-33 | 80 | 100 |
| I-34 | 80 | 100 |
| I-35 | 80 | 100 |
| I-36 | 80 | 100 |
| I-37 | 80 | 90 |
| I-40 | 80 | 90 |
| I-41 | 80 | 90 |
| I-42 | 80 | 90 |
| I-43 | 80 | 90 |
| I-44 | 80 | 80 |
| I-46 | 80 | 90 |
| I-47 | 80 | 100 |
| I-48 | 80 | 100 |
| I-49 | 80 | 90 |
| I-50 | 80 | 90 |
| I-51 | 80 | 90 |
| I-52 | 80 | 90 |
| I-54 | 80 | 90 |
| I-55 | 80 | 90 |
| I-56 | 80 | 80 |
| I-57 | 80 | 80 |
| I-58 | 80 | 100 |
| I-59 | 80 | 90 |
| I-61 | 80 | 100 |
| I-62 | 80 | 90 |
| I-63 | 80 | 90 |
| I-64 | 80 | 90 |
| I-66 | 80 | 100 |
| I-67 | 80 | 90 |
| I-68 | 80 | 100 |
| I-69 | 80 | 100 |
| I-70 | 80 | 90 |
| I-71 | 80 | 90 |
| I-72 | 80 | 100 |
| I-73 | 80 | 100 |
| I-74 | 80 | 90 |
| I-75 | 80 | 90 |
| I-76 | 80 | 90 |
| I-77 | 80 | 90 |
| I-78 | 80 | 90 |
| I-79 | 80 | 100 |
| P-01 | 80 | 100 |
| P-02 | 80 | 100 |
| P-03 | 80 | 100 |
| P-04 | 80 | 100 |
| P-05 | 80 | 100 |
| P-06 | 80 | 100 |
| P-07 | 80 | 100 |
| P-08 | 80 | 100 |
| P-09 | 80 | 100 |
| P-100 | 80 | 100 |
| P-101 | 80 | 100 |
| P-102 | 80 | 90 |
| P-103 | 80 | 90 |
| P-11 | 80 | 100 |
| P-12 | 80 | 100 |
| P-13 | 80 | 100 |
| P-14 | 80 | 100 |
| P-15 | 80 | 100 |
| P-16 | 80 | 100 |
| P-17 | 80 | 100 |
| P-18 | 80 | 100 |
| P-19 | 80 | 100 |
| P-20 | 80 | 100 |
| P-21 | 80 | 100 |
| P-22 | 80 | 100 |
| P-23 | 80 | 90 |
| P-24 | 80 | 100 |
| P-25 | 80 | 100 |
| P-26 | 80 | 100 |
| P-27 | 80 | 100 |
| P-28 | 80 | 100 |
| P-29 | 80 | 100 |
| P-30 | 80 | 100 |
| P-31 | 80 | 100 |
| P-32 | 80 | 100 |
| P-33 | 80 | 100 |
| P-34 | 80 | 90 |
| P-35 | 80 | 100 |
| P-37 | 80 | 100 |
| P-46 | 80 | 100 |
| P-47 | 80 | 100 |
| P-48 | 80 | 100 |
| P-49 | 80 | 100 |
| P-50 | 80 | 100 |
| P-51 | 80 | 100 |
| P-52 | 80 | 100 |
| P-53 | 80 | 100 |
| P-54 | 80 | 100 |
| P-55 | 80 | 100 |
| P-56 | 80 | 100 |
| P-57 | 80 | 100 |
| P-58 | 80 | 100 |
| P-59 | 80 | 100 |
| P-60 | 80 | 100 |
| P-61 | 80 | 100 |
| P-62 | 80 | 90 |
| P-63 | 80 | 100 |
| P-64 | 80 | 100 |
| P-65 | 80 | 100 |
| P-66 | 80 | 100 |
| P-67 | 80 | 90 |
| P-68 | 80 | 90 |
| P-69 | 80 | 90 |
| P-70 | 80 | 80 |
| P-71 | 80 | 80 |
| P-72 | 80 | 80 |
| P-75 | 80 | 100 |
| P-76 | 80 | 100 |
| P-79 | 80 | 90 |
| P-80 | 80 | 90 |
| P-81 | 80 | 100 |
| P-82 | 80 | 100 |
| P-83 | 80 | 100 |
| P-84 | 80 | 80 |
| P-85 | 80 | 90 |
| P-86 | 80 | 100 |
| P-87 | 80 | 100 |
| P-88 | 80 | 100 |
| P-89 | 80 | 90 |
| P-90 | 80 | 100 |
| P-94 | 80 | 90 |
| P-95 | 80 | 90 |
| P-97 | 80 | 100 |
| P-98 | 80 | 100 |
| P-99 | 80 | 90 |

TABLE 22
Post-emergence action at 80 g/ha against SETVI in %

| Example | Dosage | |
|---|---|---|
| number | [g/ha] | SETVI |
| I-01 | 80 | 90 |
| I-02 | 80 | 100 |
| I-03 | 80 | 100 |
| I-04 | 80 | 100 |
| I-05 | 80 | 100 |
| I-06 | 80 | 100 |
| I-07 | 80 | 100 |
| I-08 | 80 | 90 |
| I-09 | 80 | 100 |
| I-10 | 80 | 100 |
| I-11 | 80 | 100 |
| I-12 | 80 | 100 |
| I-13 | 80 | 100 |
| I-14 | 80 | 90 |
| I-15 | 80 | 100 |
| I-16 | 80 | 100 |
| I-17 | 80 | 100 |
| I-18 | 80 | 100 |
| I-19 | 80 | 100 |
| I-20 | 80 | 100 |
| I-21 | 80 | 100 |
| I-31 | 80 | 100 |
| I-33 | 80 | 100 |
| I-34 | 80 | 100 |
| I-37 | 80 | 100 |
| I-40 | 80 | 100 |
| I-41 | 80 | 100 |
| I-42 | 80 | 100 |
| I-43 | 80 | 100 |
| I-44 | 80 | 100 |
| I-45 | 80 | 100 |
| I-46 | 80 | 100 |
| I-50 | 80 | 100 |
| I-51 | 80 | 100 |
| I-52 | 80 | 100 |
| I-54 | 80 | 100 |
| I-55 | 80 | 100 |
| I-56 | 80 | 100 |
| I-57 | 80 | 100 |
| I-58 | 80 | 100 |
| I-59 | 80 | 100 |
| I-61 | 80 | 100 |
| I-62 | 80 | 100 |
| I-63 | 80 | 100 |
| I-64 | 80 | 100 |
| I-66 | 80 | 100 |
| I-67 | 80 | 90 |
| I-68 | 80 | 100 |
| I-70 | 80 | 100 |
| I-71 | 80 | 100 |
| I-72 | 80 | 100 |
| I-73 | 80 | 100 |
| I-74 | 80 | 100 |
| I-75 | 80 | 100 |
| I-76 | 80 | 100 |
| I-77 | 80 | 100 |
| I-78 | 80 | 100 |
| I-79 | 80 | 100 |
| P-01 | 80 | 100 |
| P-02 | 80 | 100 |
| P-03 | 80 | 100 |
| P-04 | 80 | 100 |
| P-05 | 80 | 100 |
| P-06 | 80 | 100 |
| P-07 | 80 | 100 |
| P-08 | 80 | 100 |
| P-09 | 80 | 100 |
| P-100 | 80 | 100 |
| P-101 | 80 | 100 |
| P-102 | 80 | 90 |
| P-103 | 80 | 100 |
| P-104 | 80 | 100 |
| P-11 | 80 | 100 |
| P-12 | 80 | 100 |
| P-13 | 80 | 100 |
| P-14 | 80 | 100 |
| P-15 | 80 | 100 |
| P-16 | 80 | 100 |
| P-17 | 80 | 100 |
| P-18 | 80 | 100 |
| P-19 | 80 | 100 |
| P-20 | 80 | 100 |
| P-21 | 80 | 100 |
| P-22 | 80 | 100 |
| P-23 | 80 | 100 |
| P-24 | 80 | 100 |
| P-25 | 80 | 100 |
| P-26 | 80 | 100 |
| P-27 | 80 | 90 |
| P-28 | 80 | 100 |
| P-29 | 80 | 100 |
| P-30 | 80 | 100 |
| P-31 | 80 | 100 |
| P-32 | 80 | 100 |
| P-33 | 80 | 100 |
| P-34 | 80 | 100 |
| P-35 | 80 | 100 |
| P-36 | 80 | 100 |
| P-37 | 80 | 100 |
| P-46 | 80 | 100 |
| P-47 | 80 | 100 |
| P-48 | 80 | 100 |
| P-49 | 80 | 100 |
| P-50 | 80 | 100 |
| P-55 | 80 | 100 |
| P-56 | 80 | 100 |
| P-57 | 80 | 100 |
| P-59 | 80 | 100 |
| P-60 | 80 | 100 |
| P-61 | 80 | 100 |
| P-62 | 80 | 100 |
| P-63 | 80 | 100 |
| P-64 | 80 | 100 |
| P-65 | 80 | 100 |
| P-66 | 80 | 100 |
| P-67 | 80 | 100 |
| P-68 | 80 | 100 |
| P-69 | 80 | 100 |
| P-70 | 80 | 100 |
| P-71 | 80 | 100 |
| P-72 | 80 | 100 |
| P-75 | 80 | 100 |
| P-76 | 80 | 100 |
| P-77 | 80 | 100 |
| P-78 | 80 | 100 |
| P-79 | 80 | 100 |
| P-80 | 80 | 100 |
| P-81 | 80 | 100 |
| P-82 | 80 | 100 |
| P-83 | 80 | 100 |
| P-84 | 80 | 100 |
| P-85 | 80 | 100 |
| P-86 | 80 | 100 |
| P-87 | 80 | 100 |
| P-88 | 80 | 100 |
| P-89 | 80 | 100 |
| P-90 | 80 | 100 |
| P-94 | 80 | 100 |
| P-95 | 80 | 100 |
| P-97 | 80 | 100 |
| P-98 | 80 | 100 |
| P-99 | 80 | 100 |

TABLE 23
Post-emergence action at 80 g/ha against ABUTH in %

| Example | Dosage | |
|---|---|---|
| number | [g/ha] | ABUTH |
| I-02 | 80 | 80 |
| I-05 | 80 | 80 |
| I-06 | 80 | 80 |
| I-12 | 80 | 90 |
| I-18 | 80 | 80 |
| I-19 | 80 | 90 |
| I-30 | 80 | 80 |
| I-31 | 80 | 80 |
| I-32 | 80 | 80 |
| I-35 | 80 | 80 |
| I-36 | 80 | 80 |
| I-42 | 80 | 80 |
| P-03 | 80 | 80 |
| P-04 | 80 | 80 |
| P-08 | 80 | 80 |
| P-13 | 80 | 80 |
| P-14 | 80 | 90 |
| P-16 | 80 | 80 |
| P-17 | 80 | 90 |
| P-18 | 80 | 80 |
| P-23 | 80 | 80 |
| P-25 | 80 | 90 |
| P-26 | 80 | 80 |
| P-30 | 80 | 80 |
| P-32 | 80 | 90 |
| P-33 | 80 | 80 |
| P-51 | 80 | 80 |
| P-52 | 80 | 80 |
| P-53 | 80 | 80 |
| P-54 | 80 | 80 |
| P-58 | 80 | 80 |
| P-61 | 80 | 80 |
| P-62 | 80 | 80 |
| P-64 | 80 | 80 |
| P-87 | 80 | 80 |

TABLE 24
Post-emergence action at 80 g/ha against AMARE in %

| Example | Dosage | |
|---|---|---|
| number | [g/ha] | AMARE |
| I-02 | 80 | 80 |
| I-31 | 80 | 80 |
| I-36 | 80 | 80 |
| I-40 | 80 | 80 |
| I-44 | 80 | 80 |
| I-61 | 80 | 80 |
| P-18 | 80 | 80 |
| P-31 | 80 | 80 |
| P-52 | 80 | 80 |
| P-58 | 80 | 80 |
| P-62 | 80 | 80 |
| P-88 | 80 | 80 |

TABLE 25
Post-emergence action at 80 g/ha against PHBPU in %

| Example | Dosage | |
|---|---|---|
| number | [g/ha] | PHBPU |
| I-05 | 80 | 80 |
| I-41 | 80 | 80 |
| I-58 | 80 | 80 |
| I-61 | 80 | 80 |
| I-68 | 80 | 80 |
| P-81 | 80 | 80 |

TABLE 26
Post-emergence action at 80 g/ha against POLCO in %

| Example | Dosage | |
|---|---|---|
| number | [g/ha] | POLCO |
| I-05 | 80 | 100 |
| I-10 | 80 | 80 |
| I-11 | 80 | 80 |
| P-16 | 80 | 80 |
| P-51 | 80 | 80 |

TABLE 27
Post-emergence action at 80 g/ha against VIOTR in %

| Example | Dosage | |
|---|---|---|
| number | [g/ha] | VIOTR |
| I-19 | 80 | 80 |
| I-32 | 80 | 80 |
| I-47 | 80 | 100 |
| I-58 | 80 | 80 |
| I-68 | 80 | 80 |
| P-16 | 80 | 80 |
| P-17 | 80 | 80 |
| P-34 | 80 | 80 |
| P-35 | 80 | 90 |
| P-49 | 80 | 80 |
| P-71 | 80 | 100 |
| P-72 | 80 | 90 |
| P-82 | 80 | 80 |

TABLE 28
Post-emergence action at 80 g/ha against VERPE in %

| Example | Dosage | |
|---|---|---|
| number | [g/ha] | VERPE |
| I-02 | 80 | 90 |
| I-11 | 80 | 80 |
| I-30 | 80 | 80 |
| I-32 | 80 | 80 |
| I-61 | 80 | 80 |
| I-71 | 80 | 80 |
| P-21 | 80 | 80 |
| P-28 | 80 | 80 |
| P-29 | 80 | 80 |
| P-30 | 80 | 80 |
| P-52 | 80 | 80 |
| P-54 | 80 | 80 |

TABLE 29
Post-emergence action at 80 g/ha against HORMU in %

| Example | Dosage | |
|---|---|---|
| number | [g/ha] | HORMU |
| I-01 | 80 | 100 |
| I-03 | 80 | 100 |
| I-04 | 80 | 100 |
| I-06 | 80 | 100 |
| I-07 | 80 | 100 |
| I-08 | 80 | 80 |
| I-09 | 80 | 100 |
| I-12 | 80 | 100 |
| I-13 | 80 | 90 |
| I-14 | 80 | 90 |
| I-15 | 80 | 100 |
| I-16 | 80 | 100 |
| P-02 | 80 | 100 |
| P-03 | 80 | 100 |
| P-06 | 80 | 100 |
| P-07 | 80 | 100 |
| P-09 | 80 | 100 |
| P-11 | 80 | 100 |
| P-12 | 80 | 100 |
| P-14 | 80 | 100 |
| P-15 | 80 | 100 |
| P-16 | 80 | 100 |
| P-17 | 80 | 100 |
| P-18 | 80 | 100 |
| P-19 | 80 | 100 |
| P-20 | 80 | 100 |
| P-21 | 80 | 100 |
| P-22 | 80 | 100 |
| P-24 | 80 | 100 |
| P-25 | 80 | 100 |
| P-26 | 80 | 100 |
| P-27 | 80 | 90 |

For example, compounds P-22, P-25, P-26 in tables 17 to 29, at an application rate of 80 g/ha, each show 80-100% efficacy against Alopecurus myosuroides, Avena fatua, Digitaria sanguinalis, Echinochloa crus-galli, Setaria viridis and Hordeum murinum, and are therefore suitable for control of unwanted plant growth by the post-emergence method.

1. A compound comprising spirocyclopentylpyrrolin-2-one of formula (I) and/or an agrochemically acceptable salt thereof 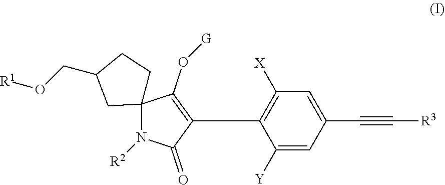
X is C₁-C₆-alkoxy, C₁-C₆-haloalkoxy or halogen,
Y is C₁-C₆-alkyl, C₁-C₆-haloalkyl, C₁-C₆-alkoxy, C₃-C₆-cycloalkyl or halogen,
R¹ is hydrogen, C₁-C₆-alkyl, C₁-C₄-alkoxy-C₂-C₄-alkyl, C₃-C₆-cycloalkyl, C₃-C₆-cycloalkyl-C₁-C₂-alkyl, C₁-C₆-haloalkyl, C₂-C₆-alkenyl, C₂-C₄-haloalkenyl, C₂-C₆-alkynyl or C₂-C₆-haloalkynyl,
R² is hydrogen, C₁-C₆-alkyl, C₁-C₄-alkoxy-C₁-C₄-alkyl, C₁-C₆-haloalkyl, C₃-C₆-cycloalkyl, C₂-C₆-alkenyl, C₂-C₆-haloalkenyl, C₂-C₆-alkynyl, C₁-C₆-alkoxy or C₁-C₆-haloalkoxy,
R³ is hydrogen, C₁-C₄-alkyl, C₁-C₄-haloalkyl, C₃-C₆-cycloalkyl or halogen,
G is hydrogen, a leaving group L or a cation E,
L is one of the radicals below 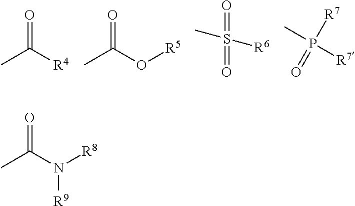
Wherein
R⁴ represents (C₁-C₄)-alkyl or (C₁-C₃)-alkoxy-(C₂-C₄)-alkyl,
R⁵ represents (C₁-C₄)-alkyl,
R⁶ represents (C₁-C₄)-alkyl, an unsubstituted phenyl or a phenyl which is mono- or polysubstituted by halogen,
(C₁-C₄)-alkyl, (C₁-C₄)-haloalkyl, (C₁-C₄)-alkoxy, (C₁-C₄)-haloalkoxy, nitro or cyano,
R⁷, R^{7′}independently of one another represent methoxy or ethoxy,
R⁸, R⁹ each independently of one another represent methyl, ethyl, phenyl or together form a saturated 5-, 6- or 7-membered ring, or together form a saturated 5-, 6- or 7-membered heterocycle having an oxygen or sulfur atom,
E represents an alkali metal ion, one ion equivalent of an alkaline earth metal, one ion equivalent of aluminum or one ion equivalent of a transition metal or a magnesium-halogen cation; represents an ammonium ion in which optionally one, two, three or all four hydrogen atoms may be replaced by identical or different radicals from the C₁-C₁₀-alkyl or C₃-C₇-cycloalkyl groups, where these independently of one another may each be mono- or polysubstituted by fluorine, chlorine, bromine, cyano, hydroxy or interrupted by one or more oxygen or sulfur atoms; represents a cyclic secondary or tertiary aliphatic or heteroaliphatic ammonium ion, optionally in each case morpholinium, thiomorpholinium, piperidinium, pyrrolidinium, or in each case protonated 1,4-diazabicyclo[1.1.2]octane (DABCO) or 1,5-diazabicyclo[4.3.0]undec-7-ene (DBU); represents a heteroaromatic ammonium cation, optionally in each case protonated pyridine, 2-methylpyridine, 3-methylpyridine, 4-methylpyridine, 2,4-dimethylpyridine, 2,5-dimethylpyridine, 2,6-dimethylpyridine, 5-ethyl-2-methylpyridine, collidine, pyrrole, imidazole, quinoline, quinoxaline, 1,2-dimethylimidazole, 1,3-dimethylimidazolium methylsulfate; or else may further represent a trimethylsulfonium ion.

2. The compound as claimed in claim 1, in which the radicals have the following meanings:
X is C₁-C₆-alkoxy, bromine, chlorine or fluorine,
Y is C₁-C₆-alkyl, C₁-C₆-haloalkyl, C₁-C₆-alkoxy or C₃-C₆-cycloalkyl,
R¹ is hydrogen, C₁-C₆-alkyl, C₁-C₄-alkoxy-C₂-C₄-alkyl, C₃-C₆-cycloalkyl, C₃-C₆-cycloalkyl-C₁-C₂-alkyl, C₁-C₆-haloalkyl, C₂-C₆-alkenyl, C₁-C₄-haloalkenyl, C₂-C₆-alkynyl or C₂-C₆-haloalkynyl,
R² is hydrogen, C₁-C₆-alkyl, C₁-C₄-alkoxy-C₂-C₄-alkyl, C₁-C₆-haloalkyl, C₃-C₆-cycloalkyl, C₂-C₆-alkenyl, C₂-C₆-haloalkenyl, C₂-C₆-alkynyl, C₁-C₆-alkoxy or C₁-C₆-haloalkoxy,
R³ is hydrogen, C₁-C₄-alkyl, C₁-C₄-haloalkyl, C₃-C₆-cycloalkyl or halogen,
G is hydrogen, a leaving group L or a cation E, where
L is one of the radicals below 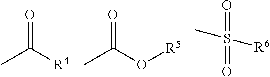
wherein
R⁴ is C₁-C₄-alkyl or C₁-C₃-alkoxy-C₁-C₄-alkyl,
R⁵ is C₁-C₄-alkyl,
R⁶ is C₁-C₄-alkyl, an unsubstituted phenyl or a phenyl which is mono- or polysubstituted by halogen, C₁-C₄-alkyl, C₁-C₄-haloalkyl or cyano,
E is an alkali metal ion, one ion equivalent of an alkaline earth metal, one ion equivalent of aluminum, one ion equivalent of a transition metal, a magnesium halogen cation or an ammonium ion, in which optionally one, two, three or all four hydrogen atoms are replaced by identical or different radicals from the C₁-C₁₀-alkyl or C₃-C₇-cycloalkyl groups, each of which are independently mono- or polysubstituted by fluorine, chlorine, bromine, cyano, hydroxy.

3. The compound as claimed in claim 1, in which the radicals have the following meanings:
X is C₁-C₆-alkoxy, bromine, chlorine or fluorine,
Y is C₁-C₆-alkyl, C₁-C₆-haloalkyl, C₁-C₆-alkoxy or C₃-C₆-cycloalkyl,
R¹ is hydrogen, C₁-C₆-alkyl, C₁-C₄-alkoxy-C₂-C₄-alkyl, C₃-C₆-cycloalkyl, C₃-C₆-cycloalkyl-C₁-C₂-alkyl, C₁-C₆-haloalkyl, C₂-C₆-alkenyl, C₁-C₄-haloalkenyl, C₂-C₆-alkynyl or C₂-C₆-haloalkynyl,
R² is hydrogen, C₁-C₆-alkyl, C₁-C₄-alkoxy-C₂-C₄-alkyl, C₁-C₆-haloalkyl, C₃-C₆-cycloalkyl, C₂-C₆-alkenyl, C₂-C₆-haloalkenyl, C₂-C₆-alkynyl, C₁-C₆-alkoxy or C₁-C₆-haloalkoxy,
R³ is hydrogen, C₁-C₄-alkyl, C₁-C₄-haloalkyl, C₃-C₆-cycloalkyl or halogen,
G is hydrogen, a leaving group L or a cation E, where
L is one of the radicals below 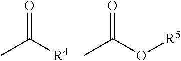
wherein
R⁴ is C₁-C₄-alkyl or C₁-C₃-alkoxy-C₁-C₄-alkyl,
R⁵ is C₁-C₄-alkyl,
E is an alkali metal ion, an ion equivalent of an alkaline earth metal, an ion equivalent of aluminum, an ion equivalent of a transition metal, a magnesium halogen cation or an ammonium ion, in which optionally one, two, three or all four hydrogen atoms are replaced by identical or different radicals from the groups C₁-C₁₀-alkyl or C₃-C₇-cycloalkyl substituted are.

4. The compound as claimed in claim 1, in which the radicals have the following meanings:
X is methoxy, ethoxy, bromine, chlorine or fluorine,
Y is methyl, ethyl, cyclopropyl, ethoxy, methoxy,
R¹ is hydrogen, ethyl, methyl, n-propyl, n-butyl, allyl, methoxymethyl or ethoxymethyl,
R² is hydrogen or methyl,
R³ is hydrogen, methyl, ethyl, bromine,
G is hydrogen, a leaving group L or a cation E, where
L is one of the radicals below 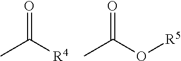
wherein
R⁴ is methyl, ethyl or i-propyl,
R⁵ is methyl, ethyl, i-propyl or t-butyl,
E is a sodium ion or a potassium ion.

5. A compound of formula (X) in which the radicals have the following meanings: 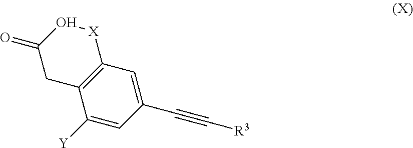
R³ is hydrogen or methyl,
X is fluorine, chlorine or bromine,
Y is methyl, ethyl, meth)oxy, ethoxy.

6. A process for preparing the compound of formula (I) an/or an agrochemically acceptable salt thereof as claimed in claim 1 comprising cyclizing a compound of formula (II) 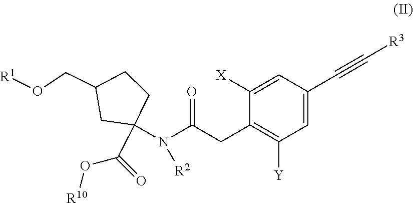
in which R¹⁰ represents alkyl, optionally methyl or ethyl, optionally in the presence of a suitable solvent or diluent, with a suitable base, by formally eliminating the R¹⁰OH group.

7. An agrochemical composition comprising a) at least one compound of formula (I) and/or an agrochemically acceptable salt thereof as defined in claim 1 and b) one or more auxiliaries and additives customary in crop protection.

8. An agrochemical composition comprising
a) at least one compound of formula (I) and/or an agrochemically acceptable salt thereof as defined in claim 1,
b) one or more active agrochemical compounds other than component a), and optionally
c) one or more auxiliaries and additives customary in crop protection.

9. A method of controlling one or more unwanted plants and/or for regulating the growth of plants, comprising applying an effective amount of at least one compound of formula (I) and/or an agrochemically acceptable salt thereof, as defined in claim 1 to the plants, seed and/or an area on which the plants grow.

10. A product comprising a compound of formula (I) and/or an agrochemically acceptable salt thereof, as defined in claim 1, as an herbicide or plant growth regulator.

11. The product as claimed in claim 10, wherein the compound of formula (I) and/or an agrochemically acceptable salt thereof is used for controlling one or more harmful plants and/or for regulating growth in one or more plant crops.

12. The product as claimed in claim 11, wherein the crop plant comprises one or more transgenic or nontransgenic crop plants.
